(19) United States
(12) Patent Application Publication
(10) Pub. No.: US 2023/0295281 A1
(43) Pub. Date: Sep. 21, 2023

(54) NATURAL ANTIBODIES IN PROPHYLAXIS AND THERAPY

(71) Applicant: VANUDIS GMBH, Stuttgart (DE)

(72) Inventors: Rudolf ÜBELHART, Bad Schonborn (DE); Torsten SCHALLER, Leimen (DE); Michaela ARNDT, Mannheim (DE)

(21) Appl. No.: 17/917,894

(22) PCT Filed: Apr. 9, 2021
(86) PCT No.: PCT/EP2021/059294

§ 371 Date: Oct. 7, 2022

(30) Foreign Application Priority Data
Apr. 10, 2020 (EP) 20169203.5
Apr. 30, 2020 (EP) 20172424.2
Sep. 9, 2020 (EP) 20195313.0

(51) Int. Cl. C07K 16/18; A61K 45/06; A61K 39/395; A61P 31/14; A61P 39/06

(52) U.S. Cl. CPC C07K 16/18; A61K 45/06; A61K 39/39533; A61P 31/14; A61P 39/06; C07K 2317/24; C07K 2317/21

(57) ABSTRACT

Described is a human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject. Moreover, described is a human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject, wherein said natural IgM and/or IgA is derived from IgM and/or IgA enriched plasma pools from healthy individuals. Further, described is a human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject, wherein said antibody is a recombinant human monoclonal natural IgM antibody. Moreover, described is a vaccine comprising a compound that induces the generation of natural IgM and/or IgA antibodies for use in a method of reducing or preventing the clinical signs or disease associated with/related to/caused by natural IgM/IgA antibody deficiency (NAD) in a subject, wherein said vaccine comprises a pharmaceutically acceptable carrier or excipient. Further, described is such a vaccine for use in a method of reducing or preventing the clinical signs or disease associated with/related to/caused by natural IgM/IgA antibody deficiency (NAD) in a subject, wherein said compound induces human natural IgM and/or IgA antibody recognizing oxidized phospholipids and/or oxidation-specific epitopes.

The present invention relates to a human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject. Moreover, the present invention relates to a human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject, wherein said natural IgM and/or IgA is derived from IgM and/or IgA enriched plasma pools from healthy individuals. Further, the present invention relates to a human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject, wherein said antibody is a recombinant human monoclonal natural IgM and/or IgA antibody. Moreover, the present invention relates to a vaccine comprising a compound that induces the generation of natural IgM and/or IgA antibodies for use in a method of reducing or preventing the clinical signs or disease associated with/related to/caused by natural IgM/IgA antibody deficiency (NAD) in a subject, wherein said vaccine comprises a pharmaceutically acceptable carrier or excipient. Further, the present invention relates to such a vaccine for use in a method of reducing or preventing the clinical signs or disease associated with/related to/caused by natural IgM/IgA antibody deficiency (NAD) in a subject, wherein said compound induces human natural IgM and/or IgA antibody recognizing oxidized phospholipids and/or oxidation-specific epitopes.

Conventional B lymphocytes, also called B2 cells or follicular (FO) B cells, originate from hematopoietic stem cells and pass through distinct definable stages during development until antigen-challenged cells terminally differentiate into immunoglobulin (antibody) secreting plasma cells or memory B cells.

In most mammals, the early stages of B2 cell development take place in the bone marrow, while the final maturation processes occur in peripheral lymphoid organs such as the spleen, lymph nodes, Peyer's Patches, etc. The most important hallmark of B lymphocytes is their B cell antigen-receptor (BCR), whose specificity is not germline encoded and thus must be individually generated during early lymphocyte development. The BCR is basically composed of a membrane-anchored version of an immunoglobulin, which is in association with the signal-transducer Ig-α/Ig-β, and co-receptors such as CD19. While a given B cell expresses an immunoglobulin with only one unique specificity, the pool of all lymphocytes together is able to recognize virtually any foreign substance.

This enormous diversity is achieved by random recombination of variable (V_{H}), diversity (D_{H}) and joining (J_{H}) gene segments at the heavy chain locus, and V_{L} and J_{L} gene segments at the light chain loci, that when assembled, encode the variable domains of the immunoglobulin. For each segment, there are multiple variants in the vertebrate genome. For instance, the human immunoglobulin heavy chain locus contains up to 51 V_{H}-, 27 D_{H}- and 6 J_{H}-gene segments plus numerous V_{L}- and J_{L}-gene segments in the light chain loci. Consequently, an immunoglobulin repertoire with roughly 3×10¹¹ different specificities can be generated by random recombination of these gene segments.

This junctional diversity can be further increased by incorporation of non-template encoded N-nucleotides at the joining sites of the gene segments by the enzyme terminal deoxynucleotidyl transferase (TdT), which is specifically expressed in early stages of B2 cell development. Since immunoglobulin gene rearrangement includes an irreversible change in the DNA sequence, all the progeny of a given activated B cell will inherit the same receptor specificity, including memory B cells that are the basis for long-term immunity against a specific pathogen.

When a mature B2 cell encounters an antigen and is thereby activated, it migrates to the B cell-T cell border within a lymphatic organ such as the lymph node or spleen, where it presents fragments of the internalized antigen via MHC class-II molecules. When a primed CD4^{pos} follicular helper (T_{FH}) T cell expresses a T cell antigen-receptor (TCR) specific for the MHC-II/antigen complex presented by the activated B cell, it provides co-stimulatory molecules such as CD40 ligand and cytokines such as interleukin 4 (IL-4), which are required to complete B cell activation. A minor fraction of the fully activated B cells rapidly differentiates to short-lived plasma cells that secrete antigen-specific IgM antibodies, also called adaptive or immune IgM. However, most activated B cells form, together with the cognate T_{FH} cells, germinal centers (GC) which are specialized structures within the lymphatic organ where affinity maturation and class-switch recombination (CSR) take place.

Affinity maturation is a process by which somatic hypermutations (SHM) are introduced within the variable regions of the immunoglobulin genes with the purpose to alter the affinity of the expressed BCR for the specific antigen. B cells expressing mutated BCRs with decreased affinity for the antigen are counterselected and die, while such B cell clones expressing a mutated BCR with improved affinity can develop further. The simultaneously ongoing process of CSR leads to the genetic rearrangement of the VDJ sequence to constant regions downstream of C-μ and C-δ, for instance to that of C-γ. Consequently, the C-μ or C-δ sequences required to express IgM and IgD isotypes, respectively, are irreversibly deleted in the genome of the respective B cells, so that the B cells produce IgG molecules containing γ-HC. At the earliest seven days later, the GC reaction generates plasma cells secreting large amounts of high-affinity IgG antibodies, which account for most of the serum IgG level, and memory B cells expressing a high-affinity IgG BCR that rapidly differentiate to IgG-secreting plasma cells upon re-encounter of the same pathogen.

Apart from the above described conventional B2 cells, another mature B cell population has been described named B1 cells. B1 cells are a small subset of B cells and because of their unique functions they are often referred to as innate-like lymphocytes.

Indeed, these cells possess several features which separate them from B2/FO B cells. Most knowledge about B1 cells and their specific functions is based on studies in mice, where they can be easily identified by the expression of a defined set of surface marker. Mouse B1 cells are typically IgM^{hi}/IgD^{low}/B220^{low}/CD23^{neg}/CD43^{pos}/Mac-1^{pos}, which contrasts with the IgM^{low}/IgD^{hi}/B220^{hi}/CD23^{pos}/CD43^{neg}/Mac-1^{neg} phenotype of naïve mature B2 cells. Based on expression of CD5, B1 cells can be further subdivided into CD5^{pos} B1a and CD5^{neg} B1 b cells. This B cell population is named B1 because it is the first B cell population that appear in ontogeny and is already present at birth, whereas the B2 cell population arises later after birth. Despite extensive investigations in the past years, it is still unclear from which progenitor cell B1 cells arise.

One hypothesis proposes that B1 cells originate from a distinct lineage committed neonatal precursor cell in the fetal liver and that this population is maintained in adults by its self-renewal capacity.

The second model suggests that B1 cells continuously arise from bone marrow-derived immature B cells that express a BCR with appropriate self-reactivity. According to the current view, enhanced BCR signaling due to recognition of self-antigen is critical for the development and maintenance of B1 cells.

Regardless of their origin, it is well-known that the B1 cell population declines with advancing age. In adult mice, B1 cells are primarily located in the peritoneal and pleural cavities and are only rarely found in lymph nodes or spleen, but they can rapidly migrate to sites of infection where they produce protective IgM antibodies without requirement for T cell help.

Importantly, and in sharp contrast to B2 cells, B1 cells possess the unique capacity to spontaneously secrete antibodies even in the absence of infection or specific immunization. In fact, even in gnotobiotic mice that were bred in strict germ-free conditions, B1 cells maintain a normal serum IgM level similarly to conventionally bred mice, for which reason these antibodies are referred to as natural antibodies (Chou, Fogelstrand et al., 2009, J Clin Invest, Vol. 119 (5)).

This contrasts to immune antibodies that are almost absent in mice bred under the same germ-free conditions. It has been estimated that in mice approximately 80% of serum IgM are natural antibodies and the remaining 20% are immune IgM. In addition, natural antibodies account for most of serum IgA antibodies. Natural antibodies differ from immune antibodies secreted by B2 cells in several aspects. For instance, natural antibodies share pre-existing germline-encoded variable domain sequences, that means they are generated by the usage of a special restricted set of D_{H}-proximal V_{H}-gene segments and in combination with particular LC genes. The genes of natural antibodies do not carry significant somatic hypermutations or N-nucleotide insertions at the joining sites due to low TdT expression in B1 cells.

Natural antibodies bind to a variety of diverse structures such as bacterial cell wall components, viruses including Influenza Virus, Vesicular Stomatitis Virus or Lymphocytic Choriomeningitis Virus, and self-antigens including phospholipids, DNA or misfolded proteins exposed by dying cells, for which reason natural antibodies are considered as auto- and polyreactive. In addition, natural antibodies have protective roles against fungal and parasitic infections.

A human B1 cell population has been described with characteristics of spontaneous IgM secretion, efficient T cell stimulation, and tonic intracellular signaling (Griffin, Holodick et al., 2011, J Exp Med, Vol. 208 (1)). Based on these criteria, a small population of B cells being present in umbilical cord blood and adult peripheral blood and expressing the unique phenotype of CD20^{pos}CD27^{pos}CD43^{pos}CD5^{pos}CD70^{neg} can be attributed to human B 1 cells which is different from the phenotype of mouse B1 cells. Interestingly, this population of B cells was particularly enriched in umbilical cord blood and found to decline with age. Of note, although a significant fraction of human B1-cell derived immunoglobulins bind to prototypical natural antibody antigens such as phosphorylcholine and a dsDNA mimotope, their V_{H} and V_{L} regions do not demonstrate a skewed gene repertoire as is the case for their mouse counterparts, and the V_{H} genes of human B1 cell immunoglobulins contain N-nucleotide additions at the V_{H}-D_{H} and D_{H}-J_{H} junctions.

Most of the knowledge about the protective role of natural antibodies against certain pathogens come from in vivo studies using genetically modified mice whose B cells are unable to secrete IgM or in which certain B cell populations such as B1a cells are lacking. These experiments revealed that mice lacking B1 cell-derived natural IgM showed significantly enhanced mortality after infection with Influenza virus, although B2 cell-derived immune IgM were still produced in response to the infection. Interestingly, the infected mice could be protected from a lethal infection by transferring serum from non-infected wild-type mice containing natural IgM, but not by serum derived from mice lacking soluble IgM. Antiviral activity of natural IgM was further demonstrated by hemagglutinin (HA) inhibition assays using bronchoalveolar lavage (BAL) fluid from uninfected mice. While IgM-containing BAL fluid showed nearly 100% neutralizing activity, depletion of IgM antibodies reduced HA inhibition by >75%. Thus, it is assumed that natural IgM protect against severe Influenza virus infections by neutralization of HA activity and IgM-induced clearance of virus particles. Natural IgM also provides an essential protection from a severe bacterial infection with e.g., Streptococcus pneumoniae, which is the most common cause of pneumonia. Studies using transgenic mice showed that the presence of B1a cells and natural IgM is crucial for surviving S. pneumoniae infection, whereas mice lacking B1a cells and natural IgM antibodies were more susceptible for lethal infection courses (Haas, Poe et al., 2005, Immunity, Vol. 23 (1)).

Interestingly, another study showed that transfer of IgG-depleted serum from naïve young mice to infected mice lacking natural IgM was protective, whereas serum from old donor mice had no beneficial effect (Holodick, Vizconde et al., 2016, J Immunol, Vol. 196 (10)). These data indicate that protective natural IgM antibody serum level decline with age, which is in line with the observed reduction in B1a cell numbers in older mice. In summary, natural IgM and possibly IgA antibodies serve as a first line defence against invading pathogens, which is important to bridge the temporal gap required to produce class-switched high-affinity immune antibodies by FO/B2 cells.

Mice deficient in secretion of IgM antibodies also show enhanced susceptibility for the development of certain diseases such as autoimmunity or atherosclerosis, which share the common feature of proinflammatory conditions independent of infectious agents (Boes, Schmidt et al., 2000, Proc Natl Acad Sci USA, Vol. 97 (3)) (Lewis, Malik et al., 2009, Circulation, Vol. 120 (5)). Similar observations have been made in human studies, which showed that patients with the autoimmune disorder systemic lupus erythematosus (SLE) contain reduced IgM antibodies in peripheral blood, although the reason for this reduction remains unclear (Senaldi, Ireland et al., 1988, Arthritis Rheum, Vol. 31 (9)).

The role of natural IgM/IgA in facilitating the clearance of apoptotic cells and cellular waste from circulation has provided a mechanistic explanation for its regulatory properties and its implication in chronic inflammation diseases. The cell-membrane of apoptotic cells is prone to oxidative damage, which results in a heterogeneous mixture of oxidized phospholipids and degradation products. Both enzymatic and non-enzymatic processes can lead to the oxidation of phospholipids, particularly of polyunsaturated fatty acids such as phosphatidylcholine, which is the main component in the cell-membrane.

Enzymatic mediators such as cytochrome P450, lipooxygenases and cyclooxygenases, and non-enzymatic components including reactive oxygen species (ROS) or free radicals derived from cellular oxygen usage in mitochondria or environmental factors such as smoking, contribute to the peroxidation reaction of the cell-membrane lipids, although the exact mechanisms still need to be resolved (Bochkov, Oskolkova et al., 2010, Antioxid Redox Signal, Vol. 12 (8)).

The continuous fragmentation of oxidized phospholipids such as oxidized phosphatidylcholine (oxPC), oxidized 1-palmitoyl-2-arachidonoyl-phosphatidylcholine (oxPAPC), oxidized cardiolipin (oxCL), oxidized phosphatidylserine (oxPS) and oxidized phosphatidylethanolamine (oxPE), results in the generation of highly reactive degradation products such as malondialdehyde (MDA) and 4-hydroxynonenal (4-HNE), which then react with amino groups exposed by lysine residues of proteins and with adjacent aminophospholipid molecules. Furthermore, 2-(ω-carboxyethyl)-pyrrole (CEP) represents an adduct between (E)-4-hydroxy-7-oxohept-5-enoic acid—an oxidative fragment of docosahexaenoic acid—and the amino groups of lysines or aminophospholipids. The resulting oxidized lipid-protein adducts and modified lipids with altered structures are neo-autoantigens, also called oxidation-specific epitopes (OSE), and represent damage-associated molecular pattern (DAMPs) which are recognized by a variety of cellular and soluble pattern-recognition receptors (PRRs) of innate immunity. OSEs were found to be prominent targets for monoclonal natural antibodies derived from mice, and it has been estimated that 20%-30% of natural IgM antibodies in mouse and human serum recognize different OSE including MDA and 4-HNE adducts, and the phosphocholine headgroup exposed by oxPC or oxPAPC (hereafter referred to as OSE-specific IgM) (Chou, Fogelstrand et al., 2009, J Clin Invest, Vol. 119 (5)).

Irrespective of the trigger, all mammalian apoptotic cells of every cellular origin display OSE on their surface, and the content of OSE increases during the ongoing apoptosis process. Apoptotic cells are recognized by OSE-specific IgM and possibly IgA antibodies and their binding induce the quiescent clearance of cellular debris. Although several soluble PRRs such as C-reactive Protein (CRP) can bind to apoptotic cells, their clearance by macrophages is fourfold reduced in the absence of IgM antibodies, indicating an essential and non-redundant role for natural IgM in this process. After binding to apoptotic cells, the pentameric IgM molecule adopts a mushroom-shaped conformation with a central protruding region in the Fc portion, which is thought to recruit C1q, a protein belonging to the complement system. C1q then cleaves and recruits other serum proteins of the complement cascade resulting in the deposition of large amounts of cleavage fragments called iC3b on the surface of apoptotic cells. Apoptotic cells are thereby opsonized with iC3b, and which can be recognized by dendritic cells and macrophages expressing specific receptors such as complement receptor 3 (CR3) or CD91 that trigger downstream intracellular signaling events resulting in ingestion of the cellular debris and production of anti-inflammatory molecules such as IL-10 and TGFβ. Clearance of apoptotic cells and cellular waste products by this mechanism is anti-inflammatory and, therefore, a minimum serum level of OSE-specific IgM is required to maintain normal tissue homeostasis.

However, in certain situations, the balance between the formation and the clearance of OSE by innate immune mechanisms is lost owing to increased apoptosis rates or oxidative stress, or by dysfunctional immune functions such as reduced serum levels of OSE-specific IgM antibodies. In such a scenario, apoptotic cells presenting high amounts of OSE accumulate in tissue or the vascular wall, where they release intracellular contents normally not found in circulation and which have the propensity to trigger both innate and adaptive immune responses and thereby contribute to chronic inflammation processes.

Notably, also phospholipids present in low-density lipoprotein (LDL) can be modified by oxidative stress leading to formation of OSE, which are similar to those generated in the cell-membrane of apoptotic cells. The role of OSEs as initiators of chronic inflammation responses has been well documented for the cardiovascular disease atherosclerosis. In these patients, high concentrations of different species of OSE exposed on oxidized LDL (oxLDL) are characteristically found in the intima of medium and large sized arteries, where they bind to cellular PRRs such as SRA-I, SRA-II, SRB-I, CD36, TLR-4 and TLR-6 expressed by macrophages located in the subendothelial space. Sensing of oxLDL by the heterotrimeric CD36/TLR-4/TLR-6 receptor complex leads to uncontrolled uptake by macrophages, inflammasome priming through NFκB signaling and the release of proinflammatory cytokines such as IL-6, TNFα and IL-113 and chemoattractants such as CCL2 and CXCL8, causing recruitment and activation of additional monocytes and T cells to the lesion and the establishment of the proinflammatory environment (Stewart, Stuart et al., 2010, Nat Immunol, Vol. 11 (2)). The enhanced uptake of oxLDL leads to deposition of cholesterol-rich lipids in intracellular endosomes and the transformation of the macrophages into so-called foam cells being considered as hallmark of atherosclerotic lesion development. Continuous uptake of oxLDL combined with an inefficient degradation of intracellular cholesterol can lead to formation of cholesterol crystals that damage the lysosomal membranes, which then activates the NOD/LRR/Pyrin domain-containing protein 3 (NLRP3) inflammasome complex, thereby further enhancing secretion of IL-1β and the propagation of the inflammation response. Not only macrophages, also endothelial cells can sense OSE through the cellular PRR LOX-1, which leads to oxLDL uptake and accumulation of cholesterol in intracellular compartments. The release of cholesterol into the cytoplasm due to damage of lysosomal membranes induces an endoplasmatic reticulum (ER) stress response leading to apoptosis of the respective macrophages and endothelial cells.

Consequently, this leads to the development of plaques containing an acellular necrotic core which is filled with cellular debris and lipid gruel, and which is capped by a fibrous lattice structure. When these plaques erode or rupture, the released material contacts the circulating coagulation system and thereby triggers thrombus formation which may result in severe clinical manifestations such as myocardial infarction or stroke.

In healthy individuals, OSE displayed on oxLDL are bound by OSE-specific IgM and possibly IgA antibodies, thereby shielding oxLDL from binding to cellular PRR expressed on macrophages, which prevents uncontrolled uptake of oxLDL, formation of foam cells and pro-inflammatory responses. In addition, OSE-specific IgM and possibly IgA antibodies limit the accumulation of apoptotic cells in developing lesions through recognition of OSE on the surface of apoptotic cells and induction of their quiescent clearance in a C1q-dependent manner.

Following this explanation, individuals with reduced serum OSE-specific IgM and possibly IgA level are expected to be predisposed for the development of cardiovascular diseases (CVD). In fact, several studies in humans have shown that plasma levels of OSE-specific IgM are inversely correlated with the risk to develop CVD. For instance, the concentration of MDA-LDL-specific IgM antibodies are inversely correlated with the carotid intima-media thickness or the risk of developing a >50% diameter stenosis in the coronary arteries (Karvonen, Paivansalo et al., 2003, Circulation, Vol. 108 (17)) (Tsimikas, Brilakis et al., 2007, J Lipid Res, Vol. 48 (2)). In line with these observations, IgM titer to oxPC have been reported to be inversely correlated with the incidence of heart attack or stroke (Fiskesund, Stegmayr et al., 2010, Stroke, Vol. 41 (4)) (Gronlund, Hallmans et al., 2009, Eur J Cardiovasc Prev Rehabil, Vol. 16 (3)). Thus, these observations indicate that OSE-specific IgM antibodies may have properties to reduce the risk to develop CVD, although the mechanisms are not entirely resolved.

The accumulation of OSE in diseased tissues have also been found in patients suffering from other sterile inflammation diseases, such as sterile acute lung injury (ALI), age-related macular degeneration (AMD), multiple sclerosis (MS) or Alzheimer's Disease (AD) (Weismann and Binder, 2012, Biochim Biophys Acta, Vol. 1818 (10)). In addition, OSEs also accumulate in a wide variety of acute situations induced by pathogen infections. For instance, the avian Influenza virus H5N1 leads to ALI in the lungs of infected mice, and the lung tissue is characterized by a high content of oxidized phospholipids and OSEs (Imai, Kuba et al., 2008, Cell, Vol. 133 (2)).

The major source of phospholipids in the lung is surfactant, which forms a film at the alveolar liquid-air interface and reduces surface tension. Surfactant contains up to 90% phospholipids including those with polyunsaturated fatty acids that can be oxidized. The BAL fluid of H5N1 Influenza virus-infected mice contains OSEs such as oxPAPC and MDA adducts that stimulate the secretion of high amounts of the pro-inflammatory cytokine IL-6 by alveolar macrophages via the TLR4-TRIF-TRAF6 signaling pathway. Interestingly, the transfer of purified synthetically oxidized surfactant phospholipids into the lungs of non-infected healthy wildtype mice is sufficient to induce strong alveolar macrophage activation and ALI, and IL-6 production in response to surfactant oxidized phospholipids can be reduced by the mouse monoclonal antibody E06 that recognizes the phosphocholine headgroup of oxPC and oxPAPC. Notably, both phosphocholine-exposed oxPC and MDA adducts can be found in the lung tissues of patients who have died from acute respiratory distress syndrome (ARDS)—the most severe form of ALI—induced by H5N1 and SARS coronavirus (CoV) infections. In addition, the formation of oxidized phospholipids and OSE can also be found in lung tissues of Anthrax-infected Rhesus monkeys, Anthrax-infected rabbits, Monkey Pox-infected Cynomolgus monkeys and Yersinia pestis-infected Cynomolgus monkeys. Thus, across multiple species, infections with various lethal lung pathogens such as H5N1 Influenza virus, SARS-CoV, Anthrax, Y. pestis, or Monkey Pox virus triggers OSE formation in the lung. These observations show that oxidative stress and the accumulation of OSE are an important driver of macrophage activation and ALI across species.

The recently described novel coronavirus SARS-CoV-2 is responsible for an ongoing word-wide pandemic outbreak of atypical pneumonia (COVID-19) and as of Apr. 5, 2020 has infected more that 1.4 million people, killing more than 65.000 of them in >200 countries and territories. The overall death rate of SARS-CoV-2 infections is >5% and most patients who died developed ARDS.

In light of the prior art, there is a need to provide further means and methods for the effective treatment or prevention of a disorder or a disease caused by coronavirus SARS-CoV-2.

The present invention is based on the surprising observation that in SARS-CoV-2 infected individuals the probability for development of severe symptoms and ARDS is largely depend on risk factors such as advanced age and underlying medical conditions including CVD, diabetes, respiratory disease or hypertension. In addition, epidemiological data indicate relative protection from severe COVID-19 disease in female versus male populations and individuals who have been vaccinated for e.g., pneumococcus or tuberculosis (e.g. w/BCG): Importantly, key pathologic findings of ARDS in SARS-CoV-2 infected patients appear to be similar to H5N1 Influenza virus and SARS-CoV infections and are characterized by accumulation of inflammatory cells, edema formation, and marked increase in pro-inflammatory cytokines such as IL-18, IL-6, IL-8 and TNFα resembling a cytokine storm profile. This indicates a common trait in ultimate immune surveillance failure in infected individuals independent from the pathogen.

Thus, the present invention is based on the proposal that, as a common feature for eventual host immune failure in a variety of infectious diseases massive formation of oxidized phospholipids and OSE accumulate in the lung of also SARS-CoV-2 infected patients that trigger pro-inflammatory cytokine production in macrophages and thereby initiate the deterioration phase in COVID-19 (and other infectious diseases).

In terms of the present invention, high levels of circulating OSE-specific IgM and possibly IgA antibodies confer protection because they bind to oxidized phospholipids and OSE and thereby promote their save clearance using the C1q pathway accompanied by the production of anti-inflammatory cytokines such as IL-10 and TGFβ by phagocytes, which in turn counteracts activation of alveolar macrophages and induction of fatal cytokine storm syndrome and ARDS.

A possible implication of OSE-specific IgM/IgA antibodies in the pathology of COVID-19 disease is supported by several observations.

Firstly, patients with advanced age (>60 years) have a dramatically higher mortality rate (up to 30%) compared to younger patients (0-29 years) with a mortality rate below 1%. The higher propensity of elderly patients to develop severe ALI and ARDS is consistent with the observation that human B1 cells and B1 cell-derived natural antibodies decline with age (Griffin, Holodick et al., 2011, J Exp Med, Vol. 208 (1)) (Rodriguez-Zhurbenko, Quach et al., 2019, Front Immunol, Vol. 10)).

Secondly, patients with underlying medical conditions such as CVD have significantly higher mortality rates (>10%) compared to patients without pre-existing conditions. As described above, the propensity to develop CVD is inversely associated with serum level OSE-specific IgM antibodies, which also is a risk factor to develop severe ALI and ARDS. This correlation is additionally supported by the fact that men show more severe infection courses and a higher mortality rate compared to women, consistent with higher IgM serum level detected in women as compared to men (Butterworth, McClellan et al., 1967, Nature, Vol. 214 (5094)) (Palmer, Schulze et al., 2015, J Dev Orig Health Dis, Vol. 6 (6)).

Taken together, these observations indicate an inverse correlation of serum level of OSE-specific IgM and possibly IgA antibodies and severity of COVID-19, and, accordingly, in terms of the present invention, administration of OSE-specific IgM and possibly IgA antibodies to COVID-19 patients a highly effective method to ameliorate ALI and ARDS induced by SARS-CoV-2 (and other) infection, or protect infected individuals from developing severe symptoms such as ALI and ARDS.

In summary, the here described pro-inflammatory diseases, that are either pathogen-independent chronic or infection-induced acute conditions, share the common feature of a high burden of oxidized phospholipids and OSE in the inflamed tissues as a result from reduced levels of OSE-specific IgM and possibly IgA antibodies.

In apparently very different diseases, the extensive accumulation of OSE shift the balance from the quiescent clearance of OSE-presenting debris or molecules towards sensing of OSE by cellular PRR expressed by macrophages resulting in their activation and the release of high amounts of pro-inflammation cytokines such as IL-6. To interfere with this inflammatory pathway, the administration of OSE-specific antibodies of the IgM and/or of the IgA isotype, or plasma pools enriched for these, into affected patients restores, in terms of the present invention, homeostatic conditions by facilitating clearance of OSE-bearing cellular debris or molecules such as oxLDL by C1q-dependent mechanisms, which is accompanied by production of anti-inflammatory cytokines such as IL-10 and TGFb by the phagocytic cells.

The finding of the present invention is all the more surprising because it is known to the skilled person in the art that OxPL and OSE exhibit anti-inflammatory and protective effects in the context of sepsis and acute injuries.

In fact, as already mentioned above, the anti-inflammatory effects of oxPL and OSE depend on their concentrations and include (1) inhibition of “sterile” acute lung injury induced by viral- and bacterial-derived inflammatory mediators (Ma et al., 2004, Am J Physiol Lung Cell Mol Physiol. 286:808-816; Nonas et al., 2006, Am J Respir Crit Care Med. 173:1130-1138); (2) inhibition of “aseptic” acute lung injury induced by injurious mechanical ventilation, and therefore it has been suggested that the use of 1-palmitoyl-2-(5,6-epoxyisoprostane E2)-sn-glycero-3-phosphorylcholine (PEIPC)- and 1-palmitoyl-2-(5,6-epoxycyclopentenone)-sn-glycero-3-phosphorylcholine (PECPC)-like stabilized compounds may show beneficial effects in other “aseptic” lung injury models such as ischemia/reperfusion (Nonas et al., 2008, Crit Care. 12:R27); and (3) inhibition of lung vascular leak and inflammation in the secondary acute lung injury induced by acute necrotizing pancreatitis (Li et al., 2007, Pancreas. 35:27-36).

These anti-inflammatory effects are mediated by enhanced endothelial barrier function (Birukov et al., 2004, Circ Res. 95:892-901; Birukova et al., 2007, Am J Physiol Lung Cell Mol Physiol. 292:924-935), induction of signaling pathways that lead to upregulation of anti-inflammatory genes, inhibition of pro-inflammatory gene expression (Eligini et al., 2002, Cardiovasc Res. 55:406-415; Ma et al., 2004, Am J Physiol Lung Cell Mol Physiol. 286:808-816; Otterbein et al., 2000, Nat Med. 6:422-428; Otterbein et al., 2003, Nat Med. 9:183-190), and prevention of the interaction of pro-inflammatory bacterial products with host cells (Bochkov et al., 2002, Nature. 419:77-81; Walton et al., 2003, Arterioscler Throm Vasc Biol. 23:1197-1203). However, in patients experiencing ARDS induced by infections with lung pathogens such as SARS-CoV-2, SARS-CoV and possibly H5N1 influenza viruses, we propose that multiple mechanisms implicated in the formation of ROS and oxidative stress convene in lungs of affected patients where oxPL and OSE accumulate to concentrations high enough to promote the biological effects described in the present invention.

In light of this general principle, the present invention not only provides further means and methods for the treatment or prevention of a disorder or a disease caused by coronavirus SARS-CoV-2, in particular (severe) symptoms such as ALI and ARDS.

Rather, the above general principle applies, in more general terms of the present invention, to the treatment or prevention of a disorder or a disease associated with/related to/caused by a natural IgM/IgA antibody deficiency (NAD) in a subject.

Accordingly, in accordance with the present invention, the subgroup of natural IgM and/or IgA antibodies, preferably OSE-specific natural IgM and/or IgA antibodies, is used in the treatment or prevention of a disorder or a disease associated with/related to/caused by a natural IgM/IgA antibody deficiency (NAD) in a subject.

In view of the prior art, the technical problem underlying the present invention is the provision of further means and methods for the treatment or prevention of a disorder or a disease associated with/related to/caused by a natural IgM/IgA antibody deficiency (NAD) in a subject.

The technical problem is solved by provision of the embodiments characterized in the claims.

Therefore, the present invention relates to a human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject.

More specifically, in a first aspect, the present invention relates to a human or humanized natural IgM and/or IgA antibody recognizing oxidized phospholipids and/or oxidization-specific epitopes for use in a method of treating or preventing a disorder or a disease associated with/related to/caused by a natural IgM/IgA antibody deficiency (NAD) in a subject, wherein said natural IgM and/or IgA is derived from IgM and/or IgA enriched plasma pools from healthy individuals.

In a second aspect, the present invention relates to a human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject, wherein said antibody is a recombinant human monoclonal natural IgM antibody.

In a third aspect, the present invention relates to a vaccine comprising a compound that induces the generation of natural IgM and/or IgA antibodies for use in a method of reducing or preventing the clinical signs or disease associated with/related to/caused by natural IgM/IgA antibody deficiency (NAD) in a subject, wherein said vaccine comprises a pharmaceutically acceptable carrier or excipient. Preferably, the present invention relates to such a vaccine for use in a method of reducing or preventing the clinical signs or disease associated with/related to/caused by natural IgM/IgA antibody deficiency (NAD) in a subject, wherein said compound induces human natural IgM and/or IgA antibody recognizing oxidized phospholipids and/or oxidation-specific epitopes.

As mentioned above, it has surprisingly been described in the present application that a subgroup of natural IgM and/or IgA antibodies can be used in the treatment or prevention of a disorder or a disease associated with/related to/caused by a natural IgM/IgA antibody deficiency (NAD) in a subject, i.e., human or humanized natural IgM and/or IgA antibody recognizing oxidized phospholipids and/or oxidation-specific epitopes.

Thus, the present invention generally relates to a human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject.

A “natural IgM and/or IgA antibody” in terms of the present invention is an antibody of the IgM and/or IgA isotype present in the serum of a healthy animal or human subject, which is produced by B1 cells independently of infection or immunization, and which possesses auto- and/or polyreactive antigen binding capacities.

Although the present invention is focused on “natural IgM and/or IgA antibodies”, whenever reference is made herein to “natural antibodies”, this term does not only relate to “natural IgM and/or IgA antibodies” but may also relate to “natural IgG”, in particular IgG2.

The human or humanized natural IgM and/or IgA antibody of the present invention is furthermore characterized in that it recognizes oxidized phospholipids and/or oxidation-specific epitopes and are, accordingly, a subgroup of the natural IgM and/or IgA repertoire of a subject.

Thus, the human or humanized natural IgM and/or IgA antibody of the present invention recognizing oxidized phospholipids and/or oxidation-specific epitopes is a subgroup/subfraction/subpopulation of the natural IgM and/or IgA repertoire of a subject. The terms “subfraction” and “subpopulation” will be used in the following as equivalent of the term “subgroup”.

In a preferred embodiment, the human or humanized natural IgM and/or IgA antibody of the present invention recognizing oxidized phospholipids and/or oxidation-specific epitopes is a subgroup/subfraction/subpopulation of the natural IgM and/or IgA repertoire from IgM and/or IgA enriched plasma pools from healthy individuals as described herein. Said IgM and/or IgA enriched plasma pools from healthy individuals are preferably themselves enriched for natural IgM and/or IgA antibodies which recognize oxidized phospholipids and/or oxidation-specific epitopes.

Methods for enriching antibodies which specifically recognize oxidized phospholipids and/or oxidation-specific epitopes are known to the skilled person and can routinely be applied. For example, antibody affinity purification methods can be applied to enrich a sample for specific antibodies. Antigen-specific affinity-affinity purification methods of only those antibodies in a sample that bind to a particular antigen molecule (i.e., oxidized phospholipids and/or oxidation-specific epitopes in accordance with the present invention) through their specific antigen-binding domains are known to the skilled person.

In preferred embodiments, the enrichment for IgM and/or IgA antibodies recognizing oxidized phospholipids and/or oxidation-specific epitopes means that:

the concentration of the IgM and/or IgA antibodies recognizing oxidized phospholipids and/or oxidation-specific epitopes is more than 5% higher compared to the concentration in the total IgM and/or IgA repertoire of a subject, preferably more than 10%, 20%, 30%, 40%, 50%, 60% or 70% or even more than 80%, 90%, 95% or 99%. In other preferred embodiments, the concentration of the IgM and/or IgA antibodies recognizing oxidized phospholipids and/or oxidation-specific epitopes is between 5% and 70% higher compared to the concentration in the total IgM and/or IgA repertoire of a subject, preferably between 10% and 70% higher, between 20% and 70% higher, between 30% and 70% higher, between 40% and 70% higher, between 50% and 70% higher, or between 60% and 70% higher.

In other preferred embodiments, the concentration of the IgM and/or IgA antibodies recognizing oxidized phospholipids and/or oxidation-specific epitopes is between 5% and 99% higher compared to the concentration in the total IgM and/or IgA repertoire of a subject, preferably between 10% and 99% higher, between 20% and 99% higher, between 30% and 99% higher, between 40% and 99% higher, between 50% and 99% higher, between 60% and 99% higher, between 70% and 99% higher, between 80% and 99% higher or between 90% and 99% higher.

In other preferred embodiments, the human or humanized natural IgM and/or IgA antibody of the present invention recognizing oxidized phospholipids and/or oxidation-specific epitopes being a subgroup/subfraction/subpopulation of the total IgM and/or IgA repertoire of a subject (and/or being a subgroup/subfraction/subpopulation of the total IgM and/or IgA repertoire from IgM and/or IgA enriched plasma pools from healthy individuals as described herein) essentially contains antibodies recognizing oxidized phospholipids and/or oxidation-specific epitopes. The term “essentially containing antibodies recognizing oxidized phospholipids and/or oxidation-specific epitopes” preferably means that the subgroup/subfraction/subpopulation is essentially pure, i.e., essentially consists of antibodies recognizing oxidized phospholipids and/or oxidation-specific epitopes.

Preferrably, the term “pure” or “essentially consisting of” means that a composition comprising said human or humanized natural IgM and/or IgA antibody of the present invention contains more than 30% antibodies recognizing oxidized phospholipids and/or oxidation-specific epitopes of the total IgM and/or IgA antibodies in said composition, preferably more than 35%, 40%, 50%, 60%, 70%, 80%, 90%, 99% or 100%.

In other preferred embodiments, the term “pure” means that a composition comprising said human or humanized natural IgM and/or IgA antibody of the present invention contains between 30% and 100% antibodies recognizing oxidized phospholipids and/or oxidation-specific epitopes of the total IgM and/or IgA antibodies in said composition, preferably between 35% and 100%, between 40% and 100%, between 50% and 100%, between 60% and 100%, between 70% and 100%, between 80% and 100%, between 90% and 100% or between 99% and 100%.

The terms “human” and “humanized” antibodies in terms of the present invention are defined further below.

The terms “recognizing”, “binding” and “binding to” as used in the context of the present invention are interchangeably used in the present invention and define a recognition and a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of oxidized phospholipids and/or oxidation-specific epitopes. Said binding/interaction is also understood to define a “specific recognition”. The term “specifically recognizing” means in accordance with this invention that the antibody is capable of specifically interacting with and/or binding to at least two molecules of each of the oxidized phospholipids and/or oxidation-specific epitopes as defined herein. Antibodies can recognize, interact and/or bind to different epitopes on oxidized phospholipids and/or oxidation-specific epitopes. This term relates to the specificity of the antibody molecule, i.e., to its ability to discriminate between the specific regions of oxidized phospholipids and/or oxidation-specific epitopes.

The term “specific interaction” as used in accordance with the present invention means that the antibody or antigen-binding fragment thereof of the invention does not or does not essentially cross-react with (poly) peptides of similar structures. Accordingly, the antibody of the invention specifically binds to/interacts with structures of oxidized phospholipids and/or oxidation-specific epitopes.

The term “oxidized phospholipids and/or oxidation-specific epitopes (also referred herein above and below as ‘OSE’)” in terms of the present invention relates to an immunogenic structure that is created by the peroxidation reaction of lipids present in mammalian cell membranes, lipoproteins such as low-density lipoprotein and high-density lipoprotein, in bacterial cell walls and/or the membrane of enveloped viruses, and that can be specifically recognized by an IgM and/or IgA antibody.

The structures of “oxidized phospholipids and/or oxidation-specific epitopes” are well-characterized and the present invention is not limited to specific “oxidized phospholipids and/or oxidation-specific epitopes”.

Preferably, said “oxidized phospholipids and/or oxidation-specific epitopes” are oxidized phosphatidylcholine, oxidized 1-palmitoyl-2-arachidonoyl-phosphatidylcholine, oxidized cardiolipin, oxidized phosphatidylserine, oxidized phosphatidylethanolamine, and terminal degradation products such as malondialdehyde (MDA), 4-hydroxynonenal (4-HNE) and 2-(ω-carboxyethyl)-pyrrole (CEP).

Cross-reactivity of a panel of antibody under investigation may be tested, for example, by assessing binding of said panel of antibody under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the oxidized phospholipid and/or oxidation-specific epitope of interest as well as to a number of more or less (structurally and/or functionally) closely related oxidized phospholipids and/or oxidation-specific epitopes. Only those constructs (i.e. antibodies, antigen-binding fragments thereof and the like) that bind to the certain structure of the oxidized phospholipid and/or oxidation-specific epitope, e.g., a specific epitope of the oxidized phospholipid and/or oxidation-specific epitope but do not or do not essentially bind to any of the other epitopes of the same oxidized phospholipid, are considered specific for the epitope or oxidized phospholipid and/or oxidation-specific epitope of interest and selected for further studies in accordance with the method provided herein. These methods may comprise, inter alia, binding studies, blocking and competition studies with structurally and/or functionally closely related molecules. These binding studies also comprise FACS analysis, surface plasmon resonance (SPR, e.g. with BIAcore®), analytical ultracentrifugation, isothermal titration calorimetry, fluorescence anisotropy, fluorescence spectroscopy or by radiolabeled ligand binding assays.

Accordingly, specificity can be determined experimentally by methods known in the art and methods as described herein. Such methods comprise, but are not limited to Western Blots, ELISA-, RIA-, ECL-, IRMA-tests and peptide scans.

The treatment or prevention of the present invention relates to the treatment or prevention of a natural IgM/IgA antibody deficiency.

“Natural IgM/IgA antibody deficiency” in this respect collectively refers to deficiencies that have in common that they lack sufficient amounts of natural ‘B1’ antibodies (particularly IgM and IgA) which (substantially) contributes to the pathogenesis of these conditions. Since NAD is considered the main driver for causing a wide variety of diseases by the same definable mechanism, namely the inability to efficiently remove oxidized specific epitope (OSE) degradation products by the innate immune system it is conceivable that methods for modulating NAD in affected patients is a valuable novel strategy for prevention and treatment of these conditions. Because this key event triggers the development of a variety of different diseases with a common cause we suggest the introduction of a novel term for this condition, Oxidized-Specific Epitope Accumulation Syndrome (OSEAS).

Accordingly, as described in the first, second and third aspect of the present invention, respectively, NAD can be modulated in 2 ways, namely i.) by the administration of natural B1 antibody preparations for therapeutic intervention or ii.) induction of B1 antibodies for disease prevention by active immunization (e.g. BCG, Pneumococcus vaccination).

Without being bound to theory, it is believed that all NADs have in common that there is a continuous fragmentation of oxidized phospholipids such as oxidized phosphatidylcholine, oxidized 1-palmitoyl-2-arachidonoyl-phosphatidylcholine, oxidized cardiolipin, oxidized phosphatidylserine and oxidized phosphatidylethanolamine which results in the generation of highly reactive degradation products such as malondialdehyde (MDA) and 4-hydroxynonenal (4-HNE), which then react with amino groups exposed by lysine residues of proteins and with adjacent aminophospholipid molecules. Furthermore, 2-(ω-carboxyethyl)-pyrrole (CEP) represents an adduct between (E)-4-hydroxy-7-oxohept-5-enoic acid—an oxidative fragment of docosahexaenoic acid—and the amino groups of lysines or aminophospholipids. The resulting oxidized lipid-protein adducts and modified lipids with altered structures are neo-autoantigens, also called oxidation-specific epitopes (OSE) (i.e., collectively referred to “oxidized phospholipids and/or oxidation-specific epitopes” in the present invention), and represent damage-associated molecular pattern (DAMPs) which are recognized by a variety of cellular and soluble pattern-recognition receptors (PRRs) of innate immunity. Apoptotic cells are recognized by oxidized phospholipid and/or oxidation-specific epitope-specific IgM antibodies in accordance with the present invention and their binding induce the quiescent clearance of cellular debris. After binding to apoptotic cells, the pentameric IgM molecule adopts a mushroom-shaped conformation with a central protruding region in the Fc portion, which is thought to recruit C1q, a protein belonging to the complement system. C1q then cleaves and recruits other serum proteins of the complement cascade resulting in the deposition of large amounts of cleavage fragments called iC3b on the surface of apoptotic cells. Apoptotic cells are thereby opsonized with iC3b, and which can be recognized by dendritic cells and macrophages expressing specific receptors such as complement receptor 3 (CR3) and CD91 that trigger downstream intracellular signaling events resulting in ingestion of the cellular debris and the production of anti-inflammatory molecules such as IL-10 and TGFβ. Clearance of apoptotic cells and cellular waste products by this mechanism is anti-inflammatory and, therefore, a minimum serum level of OSE-specific IgM is required to maintain normal tissue homeostasis.

Thus, in accordance with the present invention, high levels of circulating “oxidized phospholipids and/or oxidation-specific epitopes”-specific IgM antibodies are protective because they bind to oxidized phospholipids and OSE and thereby promote their save clearance using the C1q pathway accompanied by the production of anti-inflammatory cytokines such as IL-10 and TGFβ by phagocytes, which in turn interferes with inappropriate activation of alveolar macrophages and induction of fatal cytokine storm syndrome.

As will be outlined in more detail further below, the term “treatment” an/or “prevention” and the like are used herein to generally mean obtaining a desired pharmacological and/or physiological effect. Accordingly, the treatment of the present invention may relate to the treatment of (acute) states of a certain disease but may also relate to the prophylactic treatment in terms of completely or partially preventing a disease or symptom thereof. Preferably, the term “treatment” is to be understood as being therapeutic in terms of partially or completely curing a disease and/or adverse effect and/or symptoms attributed to the disease. “Acute” in this respect means that the subject shows symptoms of the disease. In other words, the subject to be treated is in actual need of a treatment and the term “acute treatment” in the context of the present invention relates to the measures taken to actually treat the disease after the onset of the disease or the outbreak of the disease. The treatment may also be prophylactic or preventive treatment, i.e., measures taken for disease prevention, e.g., in order to prevent the infection and/or the onset of the disease.

The antibody for use according the present invention is humanized or a human antibody, i.e., a fully human antibody. In a further preferred embodiment, the antibody for use according to the present invention is a murine antibody.

In a preferred embodiment, the antibody or the antigen-binding fragment thereof for use according the present invention is a monoclonal or a polyclonal antibody. In a further preferred embodiment, the antibody or the antigen-binding fragment thereof for use according to the present invention is a humanized or a human, i.e., a fully human antibody. In a further preferred embodiment, the antibody or the antigen-binding fragment thereof for use according to the present invention is a murine antibody.

In the context of the present invention and as described in more detail further below, the term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

The term “polyclonal antibody” as used herein, refers to an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

The term “human antibody” or the term “fully-human antibody” as used herein refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” or “murine antibody” refers to an antibody which comprises mouse/murine immunoglobulin protein sequences only. Alternatively, a “fully-human antibody” may contain rat carbohydrate chains if produced in a rat, in a rat cell, in a hybridoma derived from a rat cell. Similarly, the term “rat antibody” refers to an antibody that comprises rat immunoglobulin sequences only. Similarly, the term “rabbit antibody” refers to an antibody that comprises rabbit immunoglobulin sequences only. Similarly, in more general terms, the term “rodent antibody” refers to an antibody that comprises rodent immunoglobulin sequences only. Fully-human antibodies may also be produced, for example, by phage display which is a widely used screening technology which enables production and screening of fully human antibodies. Also human antibodies derived from phage display techniques can be used in context of this invention. Phage display methods are described, for example, in U.S. Pat. Nos. 5,403,484, 5,969,108 and 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397).

The term “chimeric antibodies”, refers to an antibody which comprises a variable region of the present invention fused or chimerized with an antibody region (e.g., constant region) from another, human or non-human species (e.g., mouse, horse, rabbit, dog, cow, chicken).

In certain aspects as described in the context of the present invention, the term antibody also relates to recombinant human antibodies, heterologous antibodies and heterohybrid antibodies. The term “recombinant human antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germ line repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

The term antibody also relates to humanized antibodies. “Humanized” forms of non-human (e.g. murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin.

Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: Jones Nature 321 (1986), 522-525; Reichmann Nature 332 (1998), 323-327 and Presta Curr Op Struct Biol 2 (1992), 593-596.

A popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. Nos. 5,225,539, 5,693,761 and 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084).

Accordingly, in context of the present invention, the term “antibody” relates to full immunoglobulin molecules as well as to parts of such immunoglobulin molecules (i.e., “antigen-binding fragment thereof”) Furthermore, the term relates, as discussed above, to modified and/or altered antibody molecules. The term also relates to recombinantly or synthetically generated/synthesized antibodies. The term also relates to intact antibodies as well as to antibody fragments thereof, like, separated light and heavy chains, Fab, Fv, Fab′, Fab′-SH, F(ab′)2. The term antibody also comprises but is not limited to fully-human antibodies, chimeric antibodies, humanized antibodies, CDR-grafted antibodies and antibody constructs, like single chain Fvs (scFv) or antibody-fusion proteins.

“Single-chain Fv” or “scFv” antibody fragments have, in the context of the invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Plückthun in The Pharmacology of Monoclonal Antibodies, Rosenburg and Moore eds. Springer-Verlag, N.Y. (1994), 269-315.

A “Fab fragment” as used herein is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody or comprising the C_{H}2, C_{H}3 and C_{H}4 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′) 2 molecule.

A “F(ab′)2 fragment” contains two light chains and two heavy chains containing a portion of the constant region between the C_{H}1 and C_{H}2 domains, such that an interchain disulfide bond is formed between the two heavy chains. A F(ab′)2 fragment thus is composed of two Fab′ fragments that are held together by a disulfide bond between the two heavy chains.

The “Fv region” comprises the variable regions from both the heavy and light chains, but lacks the constant regions.

Antibodies, antibody constructs, antibody fragments, antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook (1989), loc. cit. The term “Ig-derived domain” particularly relates to (poly) peptide constructs comprising at least one CDR. Fragments or derivatives of the recited Ig-derived domains define (poly) peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

The antibody as used in the context of the present invention for use as described above and below, is not particularly limited as long as it is an “IgM and/or IgA antibody recognizing oxidized phospholipids and/or oxidation-specific epitopes”.

Thus, the antibody may be any antibody which specifically binds to or specifically recognizes or interacts with an oxidized phospholipid and/or oxidation-specific epitope”, i.e., a domain or an antigen, preferably a surface-antigen of an oxidized phospholipid and/or oxidation-specific epitope. The skilled person is readily in a position to generate such an antibody directed to a given domain (i.e., an antigen, preferably a surface-antigen of an oxidized phospholipid and/or oxidation-specific epitope) and determine whether a respective antibody is capable of detecting/binding to a given domain, an antigen, preferably a surface-antigen of an oxidized phospholipid and/or oxidation-specific epitope.

IgA antibodies recognizing oxidized phospholipids and/or oxidation-specific epitopes can induce anti-inflammatory or proinflammatory effects, which depends on the IgA subclass and the antibody glycosylation pattern. It is known in the art that IgA2 in immune complexes acts proinflammatory on macrophages and neutrophils by binding to the Fc-alpha receptor, while IgA1 lacks such effects. Moreover, anti-inflammatory IgA1 possess more terminal sialic acid compared to IgA2, and removal of sialic acid increases the proinflammatory capacity of IgA1. In serum of healthy humans, IgA1 is predominant over IgA2 with a ratio of 9:1, whereas a shift toward the IgA2 subclass is associated with inflammation and disease activity (Steffen et al., 2020, Nat Commun, Vol. 11(1)). Thus, a natural IgA antibody in terms of the present invention is preferably an anti-inflammatory IgA1 antibody and is preferably rich in sialic acid.

In a first aspect, the above human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject in accordance with the present invention is a natural IgM and/or IgA which is derived from IgM and/or IgA enriched plasma pools from healthy individuals.

As will be outlined in more detail further below, in this first aspect, human or humanized natural IgM and/or IgA antibody recognizing oxidized phospholipids and/or oxidation-specific epitopes can be provided by naturally occurring natural IgM and/or IgA, yet preparations which are enriched for natural IgM and/or IgA.

Thus, in a preferred embodiment, the IgM and/or IgA enriched plasma pools from healthy individuals is an “intravenous normal immunoglobulin” (IVIG) product which is enriched in its IgM and/or IgA content.

“Intravenous normal immunoglobulin” (IVIG) products contain high dose pooled immunoglobulin G (IgG) and are manufactured from the plasma of a large pool of blood donors. IVIG preparation were originally used to treat infectious diseases. Since the early 1980s there have been indications that IVIG may also modulate the immune system.

In some cases of immune-mediated diseases (e.g. idiopathic thrombocytopenic purpura, Kawasaki syndrome, Guillain Barré syndrome, severe dermatomyositis, graft-vs.-host disease and septicemic shock) the use of IVIG has shown to be effective. Although the exact mode of action of IVIG in autoimmune and immunoregulatory disorders is still poorly understood, IVIg could block Fc receptors on inflammatory cells, neutralize auto-antibodies, modulate leukocyte cytokine production and block complement activation (Ott et al., J Allergy Clin Immunol. 108 (2001); Kazatchkine et al., Int Rev Immunol 5 (1989); Basta et al., J Clin Invest 84 (1989)).

Using in vitro models, such as lectin stimulation and mixed lymphocyte reaction (MLR), demonstrated that commercially available IVIG preparations interfere with activation and proliferation of immune cells as one mode of action (Nachbaur et al., Immunology 90(212) (1997); Andersson et al., Immunol Rev. 139(21) (1994); Amran et al., Clin Immunol Immunopathol 73 (180) (1994)).

Furthermore, it has been supposed that cytokine modulation by IVIG might be, at least in part, responsible for the benefits observed in human bone marrow or solid organ transplant recipients receiving IVIG (Sullivan et al., N Engl J Med 323 (1990); Peraldi et al., Transplant 62(1670) (1996)).

The two main uses for IVIG are as substitution therapy in primary or acquired antibody deficiency disorders and as therapeutic modulation of the immune system in patients with autoimmune or inflammatory conditions.

Pentaglobin® is a commercially available IVIG specifically enriched in IgM and IgA. Active ingredients of Pentaglobin® are human plasma proteins (50 mg/ml), of which at least 95% are immunoglobulins with immunoglobulin G (IgG) 38 mg (76%), immunoglobulin M (IgM) 6 mg (12%), and immunoglobulin A (IgA) 6 mg (12%). The distribution of IgG subclasses is specified in more detail and is are approximately 63% (IgG1), 26% (IgG2), 4% (IgG3), 7% (IgG4). Its content of toxin-binding and neutralizing antibodies to various Gram-positive and Gram-negative bacteria, such as Escherichia coli, Pseudomonas and Klebsiella makes it particularly suitable for treating severe bacterial infections and for substitution of immunoglobulins in patients with immune deficiency. Pentaglobin® is currently the only immunoglobulin preparation approved for this purpose. Meta-analyses of studies evaluating the efficacy of adjunctive therapy of sepsis and septic shock show that using immunoglobulins significantly reduces the mortality risk of sepsis patients. This influence is much more intensive with IgM-enriched immunoglobulin treatment than with standard immunoglobulins.

Pentaglobin® has been used in a variety of indications off-label. Viral heart disease, also known as myocarditis, is a heart condition caused by a virus. The virus attacks the heart muscle, causing inflammation and disrupting the electrical pathways that signal the heart to beat properly. Treatment with Pentaglobin® has been reported to be highly effective in resolving myocardial inflammation. Adenoviral infection was better eradicated than Parvo B19 infection (Maisch JACC, 2016, Abstract 1346 Maisch 23 Mar. 2018 Circulation. 2010; 122:A20154).

In November 2002 the severe acute respiratory syndrome (SARS) spread to all continents within several weeks and a novel coronavirus (SARS-CoV) was discovered as aetiological agent. Ho et al. from the University of Hong Kong administered Pentaglobin® to a cohort of 12 severe SARS patients who did not show favorable response to corticosteroid therapy (Ho et al., Int. J. Tuberc. Lung Dis. 8(10) (2004). After commencement of Pentaglobin® treatment there was significant improvement in radiographic scores and in oxygen requirement resulting in an uneventful recovery of ten patients after treatment. An inhibitory effect on cytokine release was discussed to be an important mechanism of action in the treatment of SARS with Pentaglobin®.

Due to its multimeric structure IgM has a higher opsonization activity, a more potent agglutination strength, a higher phagocytic activity, and a higher specific complement activation compared with monomeric IgG. These structural benefits of IgM have a decisive influence on the clinical status of patients.

Without being bound to theory, in preferred embodiments, intravenous administration of Pentaglobin® to patients infected with lung pathogens such as SARS-CoV-2, SARS-CoV, MERS-CoV, Influenza virus, Anthrax or other pathogens that induce severe ALI and ARDS, have protective effects that depend on the presence of OSE-specific IgM and/or IgA antibodies.

Thus, in a preferred embodiment, the human or humanized natural IgM and/or IgA antibody recognizing oxidized phospholipids and/or oxidation-specific epitopes are derived from Pentaglobin® which are, however, enriched for human or humanized natural IgM and/or IgA antibody vis-à-vis the Pentaglobin® preparation having 76% immunoglobulin G (IgG), 12% immunoglobulin M (IgM), and 12% immunoglobulin A (IgA) of the total plasma proteins.

Accordingly, in preferred embodiments, the natural IgM and/or IgA is derived from IgM and/or IgA enriched plasma pools from healthy individuals in that:

IgM is more than 12% of the total plasma proteins, preferably more than 13%, 15%, 20%, 30%, 40% or 50%.

In other preferred embodiments, IgM is between 13% and 15%, of the total plasma proteins, preferably between 13% and 20%, between 13% and 30%, between 13% and 40% or between 13% and 50%.

IgA is more than 12% of the total plasma proteins, preferably more than 13%, 15%, 20%, 30%, 40% or 50%.

In other preferred embodiments, IgA is between 13% and 15%, of the total plasma proteins, preferably between 13% and 20%, between 13% and 30%, between 13% and 40% or between 13% and 50%.

In another preferred embodiment, the IgM and/or IgA enriched plasma pools from healthy individuals which is an “intravenous normal immunoglobulin” (IVIG) product being enriched in its IgM and/or IgA content is the commercially available IVIG Trimodulin.

Trimodulin is an IgM concentrate derived from human blood plasma with a high content of IgG, IgM and IgA, which is currently being developed for the treatment of severe community-acquired pneumonia (sCAP). Trimodulin (IgM Concentrate) acts through a wide range of mechanisms interfering pathophysiological processes, which otherwise could lead to severe respiratory disturbances, severe sepsis, multi organ failure and ultimately death of the patient. Besides neutralisation of bacterial endotoxin and exotoxin, IgM mediates increased recognition of pathogens by certain immune cells and promotes their destruction.

More specifically, Trimodulin is a human plasma-derived native polyvalent antibody preparation for intravenous administration. Trimodulin contains immunoglobulins IgM 23%, IgA 21% and IgG 56%. Thus, active ingredients of Trimodulin are immunoglobulins with IgM 23%, IgA 21% and IgG 56% of the total plasma proteins. In a preferred embodiment, the human or humanized natural IgM and/or IgA antibody recognizing oxidized phospholipids and/or oxidation-specific epitopes are derived from Trimodulin which are, however, enriched for human or humanized natural IgM and/or IgA antibody vis-à-vis the Trimodulin preparation having 56% immunoglobulin G (IgG), 23% immunoglobulin M (IgM), and 21% immunoglobulin A (IgA) of the total plasma proteins.

Accordingly, in preferred embodiments, the natural IgM and/or IgA is derived from IgM and/or IgA enriched plasma pools from healthy individuals in that: IgM is more than 23% of the total plasma proteins, preferably more than 24%, 25%, 30%, 40% or 50%.

In other preferred embodiments, IgM is between 24% and 27%, of the total plasma proteins, preferably between 24% and 30%, between 24% and 40%, between 24% and 50% or between 24% and 60%.

IgA is more than 21% of the total plasma proteins, preferably more than 23%, 25%, 30%, 40% or 50%.

In other preferred embodiments, IgA is between 22% and 25%, of the total plasma proteins, preferably between 22% and 30%, between 22% and 40%, between 22% and 50% or between 22% and 60%.

Thus, the above human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject in accordance with the present invention is a natural IgM and/or IgA which is derived from IgM and/or IgA enriched plasma pools from healthy individuals wherein said in order to enrichment may be achieved by several means, thereby improving its protective effects:
1) The plasma can be prepared out of sera derived from young women not older than 29 years, because this population group contains the highest concentration of OSE-specific antibodies in their serum.
2) The concentration of IgM and/or IgA antibodies can be modified from 12% in the standard formulation to higher concentrations as outlined above by applying routine methods known to the skilled person to enrich IgM and/or IgA antibodies.
3) Although the concentration of OSE-specific antibodies in the product is unknown, the product formulation can be further enriched for OSE-specific IgM and IgA antibodies by applying routine methods known to the skilled person to enrich corresponding specific antibodies.

Thus, as mentioned above, the human or humanized natural IgM and/or IgA antibody of the present invention recognizing oxidized phospholipids and/or oxidation-specific epitopes is preferably a subgroup/subfraction/subpopulation of the natural IgM and/or IgA repertoire of a subject which is enriched/purified for antibodies recognizing oxidized phospholipids and/or oxidation-specific epitopes. Accordingly, as regards said enrichment and/or purification for antibodies recognizing oxidized phospholipids and/or oxidation-specific epitopes, the same applies, mutatis mutandis, to the present first aspect of the present invention as has been set forth above in the context of the more general disclosure of the present invention.

In another preferred embodiment, the above human or humanized natural IgM and/or IgA antibody recognizing oxidized phospholipids and/or oxidation-specific epitopes which are enriched for human or humanized natural IgM and/or IgA antibody are derived from plasma of subjects which have been vaccinated with a vaccine according to the third aspect of the present invention as described further below. In a preferred embodiment, said vaccine is a Pneumococcus or Bacillus Calmette-Guérin (BCG) vaccine. In another preferred embodiment, said subject is a young woman not older than 25 years.

In a preferred embodiment, said disorder or a disease associated with/related to/caused by natural IgM/IgA antibody deficiency (NAD) in accordance with the first aspect of the present invention, is a natural antibody deficient infectious, neurodegenerative, metabolic, autoimmune, or cardiovascular disease.

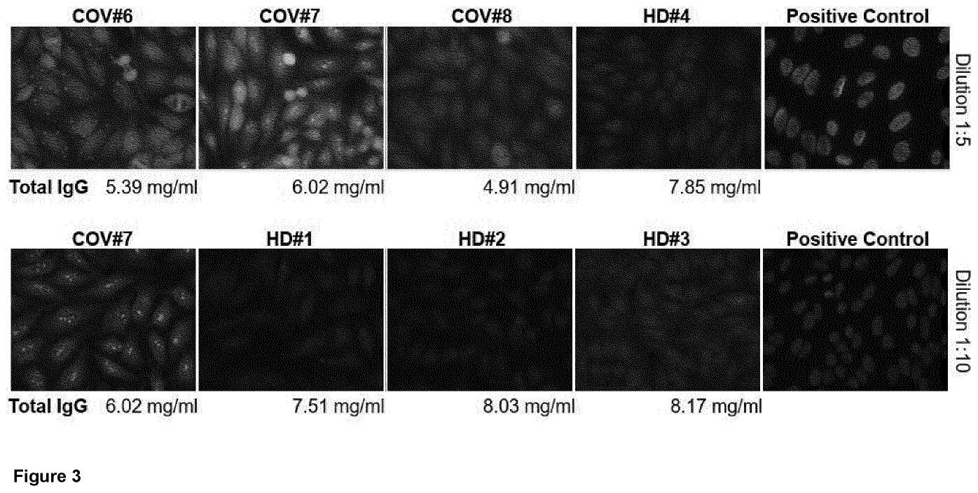

In fact, as regards autoimmune diseases, it has surprisingly been found in the present invention (see Example 22 and FIG. 3) that in patients with severe COVID-19, autoimmune IgG antibodies are generated. These data support that the lack of natural antibodies (nABs) in terms of the present invention can result in the development of autoimmune antibodies during severe COVID-19 courses. The presence of these autoimmune antibodies provides evidence for recurring or long-lasting COVID-19 disease symptoms, supporting that sufficient levels of natural antibodies, provision of (monoclonal) natural IgMs or IgAs, or preparations enriched for natural antibodies (e.g. Pentaglobin®) in terms of the present invention can prevent the generation or reduce the levels of autoimmune antibodies.

In another preferred embodiment, the human or humanized natural IgM and/or IgA antibody recognizing oxidized phospholipids and/or oxidation-specific epitopes is for use in a method of treating or preventing a disorder or a disease associated with/related to/caused by a natural IgM/IgA antibody deficiency (NAD) in a subject, wherein said disorder or a disease associated with/related to/caused by natural IgM/IgA antibody deficiency (NAD) is the virus infection disease COVID-19 caused by the β-Coronavirus SARS-CoV-2.

The term “SARS-CoV-2” comprises viruses with at least 70% identity in the amino acid sequences of their expressed open reading frames to the Genebank reference sequence NC_045512.2.

More specifically, the term “SARS-CoV-2” comprises viruses with at least 70% identity in the complete genome sequence of the Genebank reference sequence NC_045512.2 (SEQ ID NO:17).

In a more preferred embodiment, the term “SARS-VoV-2” comprises a genomic sequence of SEQ ID NO:17 which is at least n % identical to the above sequence with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99.

As regards the determination of sequence identity, the following should apply: When the sequences which are compared do not have the same length, the degree of identity either refers to the percentage of nucleic acid residues in the shorter sequence which are identical to nucleic acid residues in the longer sequence or to the percentage of nucleic acid residues in the longer sequence which are identical to nucleic acid residues in the shorter sequence. Preferably, it refers to the percentage of nucleic acid residues in the shorter sequence which are identical to nucleic acid residues in the longer sequence. The degree of sequence identity can be determined according to methods well known in the art using preferably suitable computer algorithms such as CLUSTAL.

When using the Clustal analysis method to determine whether a particular sequence is, for instance, at least 60% identical to a reference sequence default settings may be used or the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

Preferably, the degree of identity is calculated over the complete length of the sequence.

In another preferred embodiment, the human or humanized natural IgM and/or IgA antibody recognizing oxidized phospholipids and/or oxidation-specific epitopes is for use in a method of treating or preventing a disorder or a disease associated with/related to/caused by a natural IgM/IgA antibody deficiency (NAD) in a subject, wherein said disorder or a disease associated with/related to/caused by natural IgM/IgA antibody deficiency (NAD) is long COVID-19.

Long COVID, also known as post-acute sequelae of SARS-CoV-2 infection, post-acute sequelae of COVID-19 (PASC), chronic COVID syndrome (CCS) and long-haul COVID is a theorized condition, proposed to be characterized by long-term sequelae-persisting after the typical convalescence period-of coronavirus disease 2019 (COVID-19). A wide range of symptoms are commonly referred to, including fatigue, headaches, shortness of breath, anosmia (loss of smell), muscle weakness, low fever, cognitive dysfunction, sleep disorders, intermittent fevers, gastrointestinal symptoms, anxiety, and/or depression.

In preferred embodiments, long COVID as referred to herein is opposed to acute COVID-19 which is defined by signs and symptoms during the first 4 weeks after infection with severe acute respiratory syndrome coronavirus 2 (SARS-CoV-2).

Thus, long COVID in terms of the present invention relates in preferred embodiments to ongoing symptomatic COVID-19 for effects from 4 to 12 weeks after onset, and/or to post-COVID-19 syndrome for effects that persist 12 or more weeks after onset.

In a preferred embodiment, the human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject in accordance with the present invention is an antibody which is capable of inhibiting the spreading of a virus from an infected cell to an adjacent second non-infected cell (cell-to-cell spread).

Cell-to-cell spread is the ability of viruses to spread to an adjacent second non-infected cell without releasing cell-free particles.

In order to examine whether an antibody is capable of inhibiting the spread of a virus from an infected cell to an adjacent second non-infected cell (cell-to-cell spread), methods well-known to the person skilled in the art can be used.

As an example, the following assay can be used: Vero cells grown to confluency on glass cover slips in 24-well tissue culture plates are infected for 4 h at 37° C. with a constant Herpes Simplex virus type 1 (HSV-1) amount of 400 TCID₅₀/well. One median tissue culture infective dose (1 TCID₅₀) is the mount of a cytopathogenic agent, such as a virus, that will produce a cytopathic effect in 50% of the cell cultures inoculated. The virus inoculum is subsequently removed, the cells washed twice with PBS and further incubated for 2 days at 37° C. in 1 ml DMEM, 2% FCS, Pen/Strep containing an excess of either natural OSE-specific IgM and/or IgA antibodies which can be derived from plasma pools or can be monoclonal antibodies or polyclonal anti-virus control serum as positive control in order to prevent viral spreading via the supernatant. Viral antigens of virus-infected cells are detected with a fluorescence labelled serum directed against the virus. Preferably, an antibody is inhibiting cell-to-cell spread if less than 20% of the adjacent cells are infected, preferably wherein less than 15%, less than 10%, less than 5%, more preferably less than 3% and most preferably less than 1% of the adjacent cells are infected in the above assay.

In a preferred embodiment, the human or humanized natural IgM and/or IgA antibody recognizing oxidized phospholipids and/or oxidation-specific epitopes for use in a method of treating or preventing a disorder or a disease associated with/related to/caused by a natural IgM antibody deficiency (NAD) in a subject in accordance with the present invention is an antibody which is capable of neutralizing the infection by a virus, preferably to thereby preventing the infection of target cells.

Neutralization is the ability of agents to prevent viruses to infect non-infected cells. In order to examine whether an antibody is capable of neutralizing a virus thereby preventing infection of a non-infected cell, methods well-known to the person skilled in the art can be used.

As an example, the following assay can be used:

Herpes Simplex virus type 1 (HSV-1) amount of 100 TCID₅₀ is incubated 1 h at room temperature with serial dilutions of either natural OSE-specific IgM and/or IgA antibodies which can be derived from plasma pools or can be monoclonal antibodies or polyclonal anti-virus control serum as positive control or DMEM, 2% FCS, Pen/Strep as second negative control. One median tissue culture infective dose (1 TCID₅₀) is the amount of a cytopathogenic agent, such as a virus, that will produce a cytopathic effect in 50% of the cell cultures inoculated.

Vero cells grown to 80% confluency in 96-well tissue culture plates are infected for 2 days at 37° C. by adding 100 μl DMEM, 2% FCS, Pen/Strep containing the virus-antibody/plasma inoculum or positive control or negative controls to the cells. The cells are further incubated for 2 days at 37° C. Cytopathic effects induced by virus infection is scored by counting wells that contain plaques and determining the infectivity of the virus-antibody/plasma pool inoculum as TCID₅₀/ml inoculum. Preferably, an antibody is neutralizing virus infection if less than 20% of infectivity, preferably less than 15%, less than 10%, less than 5%, more preferably less than 3% and most preferably less than 1% infectivity is determined compared to the negative control.

In a preferred embodiment, the human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject in accordance with the present invention is an antibody which has an anti-inflammatory activity, preferably the capability of:
reducing the accumulation of free oxidized phospholipids, preferably in infect lungs, clearing cellular debris in lung tissue,
stimulating IL-10 and/or TGFβ secretion; and/or
neutralizing pro-inflammatory immune responses triggered by cytokines.

In order to examine whether an antibody has an anti-inflammatory activity, preferably the capability of:
reducing the accumulation of free oxidized phospholipids, preferably in infect lungs,
clearing cellular debris in lung tissue,
stimulating IL-10 and/or TGFβ secretion; and/or
neutralizing pro-inflammatory immune responses triggered by cytokines,
methods well-known to the person skilled in the art can be used. As an example, the binding capacity of monoclonal or polyclonal IgM and/or IgA antibodies to oxidized phospholipids or oxidation-specific epitopes can be tested using ELISA techniques and antigens such as oxidized phospholipids and/or oxidation-specific epitopes coupled to a protein-carrier such as BSA. The binding capacity of monoclonal or polyclonal IgM and/or IgA antibodies to apoptotic cells can be tested by flow-cytometry or immunofluorescence microscopy assays using fluorescently labeled primary or secondary antibodies. To test for recruitment of C1q, apoptotic cells can be incubated with monoclonal or polyclonal IgM and/or IgA antibodies in the presence of mouse or human serum, followed by staining with fluorescently labeled C1q-specific antibodies and flow-cytometric analyses. To test for phagocytosis, equal numbers of labeled apoptotic cells and phagocytes are incubated in the presence of mouse or human serum, and in the presence or absence of OSE-specific monoclonal or polyclonal IgM and/or IgA antibodies. IgM- and/or IgA-dependent phagocytosis can then be determined using flow-cytometry. The anti-inflammatory capacity of OSE-specific IgM and/or IgA antibodies can be tested using mouse or human macrophages or monocytes that are incubated with oxidized phospholipids or oxidation-specific epitopes present on LDL, a protein-carrier such as BSA, or broncho-alveolar lavage fluid derived from mice infected with lung pathogens such as Influenza virus, in the presence or absence of OSE-specific IgM and/or IgA antibodies. The resulting activation of the macrophages or monocytes can be tested by measuring the concentration of secreted pro-inflammatory cytokines such as IL-6 using methods such as ELISA. Protective effects of OSE-specific IgM and/or IgA antibodies can be tested by intravenous injection of the antibodies into mice infected with a lethal dose of H5N1 Influenza virus or other lung pathogens. Protective effects of OSE-specific IgM and/or IgA antibodies can be tested by intravenous injection of the antibodies into Idlr−/− or apoe−/− mice that are predisposed to develop atherosclerosis.

In another preferred embodiment, the human or humanized natural IgM and/or IgA antibody recognizing oxidized phospholipids and/or oxidation-specific epitopes is for use in a method of treating or preventing a disorder or a disease associated with/related to/caused by a natural IgM/IgA antibody deficiency (NAD) in a subject, wherein said disorder or a disease associated with/related to/caused by natural IgM/IgA antibody deficiency (NAD) is an inflammatory disease or a virus infection disease.

The present invention is not particularly limited to a specific inflammatory disease or a virus infection disease. The common feature of given examples of diseases listed in this application is the accumulation of oxidized specific epitopes (OSE) from phospholipid membranes that cannot be sufficiently removed from the body. Because this key event triggers the development of a variety of different diseases with a common cause we suggest the introduction of a novel term for this condition, Oxidized-Specific Epitope Accumulation Syndrome (OSEAS).

Yet, in a preferred embodiment, said inflammatory disease is selected from the group consisting of infectious diseases mediated by respiratory viruses, preferably COVID19, influenza, MERS-COV or SARS-COV; infectious diseases caused by bacterial infections mediated by gram positive or gram negative pathogens, fungi, or parasites; and sterile inflammatory diseases, preferably cardiovascular diseases, atherosclerosis, coronary heart disease, heart attack and stroke, metabolic disorders like diabetes mellitus, neurodegenerative diseases, preferably Alzheimer's Disease, and autoimmune diseases, preferably Systemic Lupus Erythematodes, or Multiple Sclerosis.

In fact, as regards autoimmune diseases, it has surprisingly been found in the present invention (see Example 22 and FIG. 3) that in patients with severe COVID-19, autoimmune IgG antibodies are generated. These data support that the lack of natural antibodies (nABs) in terms of the present invention can result in the development of autoimmune antibodies during severe COVID-19 courses. The presence of these autoimmune antibodies provides evidence for recurring or long-lasting COVID-19 disease symptoms, supporting that sufficient levels of natural antibodies, provision of (monoclonal) natural IgMs or IgAs, or preparations enriched for natural antibodies (e.g. Pentaglobin®) in terms of the present invention can prevent the generation or reduce the levels of autoimmune antibodies.

Moreover, in another preferred embodiment, said virus infection disease is selected from the group consisting of infections by coronaviruses, preferably SARS-CoV, SARS-CoV-2, MERS); influenza viruses, parainfluenza viruses, respiratory syncytial viruses (RSV), rhinoviruses, adenoviruses, enteroviruses, human metapneumoviruses, herpesviruses, preferably HSV-1, HSV-2, VZV, EBV, HCMV, HHV-6, HHV-7, HHV-8.

In further preferred embodiment, the present invention relates to a pharmaceutical composition, comprising an effective amount of the human or humanized natural IgM and/or IgA antibody recognizing oxidized phospholipids and/or oxidation-specific epitopes for use according to the present invention and at least one pharmaceutically acceptable excipient.

The term “pharmaceutical composition” and “pharmaceutically acceptable excipient” in terms of the present invention is described in more detail further below in the context of the second aspect of the present invention which applies, mutatis mutandis, to the first aspect of the present invention as regards the terms “pharmaceutical composition” and “pharmaceutically acceptable excipient”.

In a second aspect, the above human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject in accordance with the present invention is not an IgM and/or IgA which is derived from natural occurring sources like, e.g., the above-described IgM and/or IgA enriched plasma pools from healthy individuals.

In contrast, in this second aspect, the above human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject in accordance with the present invention is a recombinant human monoclonal natural IgM or IgA antibody.

Indeed, fully human monoclonal OSE-specific antibodies of the IgM or IgA isotype may protect from pathogen-induced ALI and ARDS in accordance with the above rationale of the present invention, and, accordingly, may have beneficial effects when administered into patients with sterile inflammatory diseases such as atherosclerosis, SLE, MS, AMD and AD.

To isolate fully human OSE-specific IgM antibodies, single human B1 cells were sort-purified according to the phenotype CD20^{pos}CD27^{pos}CD43^{pos}CD5^{pos}CD70^{neg}, amplified V_{H} and V_{L} genes from single cells and tested the reactivity of the resulting recombinant antibodies.

For two clones described further below (termed “Clone 1” and “Clone 2”; see, in particular, Example 23), binding reactivities to antigens that are typical for natural antibodies including DNA, oxidized phospholipids such as oxCL and oxPC, oxLDL, MDA-LDL, Influenza virus, Lipopolysaccharide (LPS) and misfolded amyloid-β peptide oligomers. Thus, these two monoclonal natural IgM antibody clones are considered appropriate candidates for protecting patients with OSEAS related infectious, metabolic, cardiovascular, neurodegenerative, and diseases including ALI and ARDS mediating infectious diseases such as SARS-CoV-2, SARS-CoV, MERS-CoV, Influenza virus, Anthrax, or other and possibly yet unknown lung pathogens mediating severe ALI and ARDS. In addition, as described in more detail further below, these two monoclonal natural IgM antibody clones can be utilized to protect humans from the development of other sterile chronic inflammatory OSEAS mediated diseases such as atherosclerosis, SLE, DM II, MS, AMD, and AD.

However, the present invention is not limited to these two natural antibodies. Rather, the present invention relates in this second aspect to the above human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject in accordance with the present invention, wherein said antibody is, in more general terms, a recombinant human monoclonal natural IgM or IgA antibody recognizing oxidized phospholipids and/or oxidation-specific epitopes.

As already mentioned above, the term “monoclonal antibody” as used herein, refers to an antibody obtained from a population of substantially homogeneous antibodies, i.e., the individual antibodies comprising the population are very similar in sequence except for possible naturally occurring mutations that may be present in minor amounts. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies and is not to be construed as requiring production of the antibody by any particular method. As mentioned above, the monoclonal antibodies to be used in accordance with the present invention may be made by the hybridoma method described by Kohler, Nature 256 (1975), 495.

The term “recombinant” generally refers to a compound which is composed of elements which do not occur in nature in this combination.

In a preferred embodiment, the recombinant human monoclonal natural IgM and/or IgA antibody recognizing oxidized phospholipids and/or oxidation-specific epitopes for use according to the present invention is an antibody which recognizes said oxidized phospholipids and/or oxidation-specific epitopes as described above.

Preferably, the antibody binds in a polyreactive manner said oxidized phospholipids and/or oxidation-specific epitopes, preferably to, e.g., oxLDL.

In other preferred embodiments, said antibody binds to oxidized cardiolipin; viral particles (like, e.g., particles of the influenza virus); proinflammatory cytokines (e.g., TNFα) and/or miss-folded proteins.

Preferably, said antibody binds to miss-folded proteins in neurodegenerative diseases like, e.g., “oligomeric amyloid-β peptides”.

In yet another preferred embodiment, said antibody binds to oxidized phospholipids and/or oxidation-specific epitopes present in the plasma membrane of mammalian cells, in circulating lipoproteins, in the membrane of enveloped viruses or in the cell-wall of bacteria, fungi or parasites, apoptotic cells, cellular debris, to oxidized LDL, to viruses, preferably to Influenza viruses or SARS-coronaviruses, or to microbes, preferably Staphylococcus pneumoniae; misfolded proteins that accumulate in neurodegenerative diseases, preferably as oligomeric amyloid-β peptides.

In a preferred embodiment, the recombinant human monoclonal natural IgM and/or IgA antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide.

In another preferred embodiment, the recombinant human monoclonal natural IgM and/or IgA antibody is not only defined in that it recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide. The recombinant human monoclonal natural IgM and/or IgA antibody of the present invention may also be characterized in that it recognizes and binds to phosphorylcholine exposed by oxidized phosphatidylcholine and/or oxidized 1-palmitoyl-2-arachidonoyl-phosphatidylcholine but not to phosphorylcholine of non-oxidized phosphatidylcholine and non-oxidized 1-palmitoyl-2-arachidonoyl-phosphatidylcholine, to oxidized cardiolipin but not to non-oxidized cardiolipin, to oxidized phosphatidylserine but not to non-oxidized phosphatidylserine, to malondialdehyde-, 4-hydroxynonenal- and/or 2-(ω-carboxyethyl)-pyrrole-modified proteins but not to native proteins, and/or to oligomeric amyloid-β peptide but not to monomeric amyloid-β peptide.

Assays for determining whether an antibody binds to the above structures are known in the art and can, e.g., be assessed as described herein above.

In a preferred embodiment, the recombinant human monoclonal natural IgM and/or IgA antibody recognizing oxidized phospholipids and/or oxidation-specific epitopes for use according to the present invention,
comprises the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6, wherein said antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide; or
comprises the complementarity determining regions V_{H}CDR1 comprising SEQ ID NO: 9, V_{H}CDR2 comprising SEQ ID NO: 10, V_{H}CDR3 comprising SEQ ID NO: 11, V_{L}CDR1 comprising SEQ ID NO: 12, V_{L}CDR2 comprising SEQ ID NO: 13, and V_{L}CDR3 comprising SEQ ID NO:14, wherein said antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide.

Assays for determining whether an antibody binds to the above structures are known in the art and can, e.g., be assessed as described herein above.

The monoclonal antibodies which are structurally described above with reference to SEQ ID NOs: 1 to 6 and SEQ ID NOs: 9 to 14, respectively, have been characterized in Example 23, further below.

SEQ ID NOs: 1 to 6 are derived from “Clone 1”.

SEQ ID NOs: 9 to 14 are derived from “Clone 2”.

In preferred embodiments, the monoclonal antibodies described above with reference to SEQ ID NOs: 1 to 6 and SEQ ID NOs: 9 to 14, respectively, are not monospecific.

In preferred embodiments, the monoclonal antibodies described above with reference to SEQ ID NOs: 1 to 6 and SEQ ID NOs: 9 to 14, respectively, are bispecific or multispecific.

Bispecific in this context means that the monoclonal antibody specifically binds to two distinct antigens and/or epitopes of an antigen.

Multispecifc in this context means that the monoclonal antibody specifically binds to more than two distinct antigen and/or epitopes of an antigen, preferably, three, four, or five distinct antigens and/or epitopes of an antigen.

In a more preferred embodiment, said distinct antigen and/or epitope of an antigen is a danger-associated molecular pattern (DAMPs).

Damage-associated molecular patterns (DAMPs) are known in the art and are molecules within cells that are a component of the innate immune response which are released from damaged or dying cells due to trauma or an infection due to a pathogen. They are also known as danger-associated molecular patterns, danger signals, and alarmin because they serve as a warning sign for the organism to alert it of any damage or infection to its cells. DAMPs are endogenous danger signals which are discharged to the extracellular space in response to damage to the cell from trauma or pathogen.

In a particularly preferred embodiment, said danger-associated molecular pattern (DAMPs) is selected from the group consisting of OxLDL, MDA-LDL, MDA-BSA, PC-BSA and DNA.

Thus, in a particularly preferred embodiment, the monoclonal antibodies described above with reference to SEQ ID NOs: 1 to 6 and SEQ ID NOs: 9 to 14, respectively, bind to at least two danger-associated molecular pattern (DAMPs) selected from the group consisting of OxLDL, MDA-LDL, MDA-BSA, PC-BSA and DNA.

In another particularly preferred embodiment, the monoclonal antibodies described above with reference to SEQ ID NOs: 1 to 6 (corresponding to “Clone 1”) bind to the danger-associated molecular pattern (DAMPs) OxLDL, MDA-LDL, MDA-BSA, PC-BSA and DNA. Thus, it is multispecific in terms of the present invention.

In another even more preferred embodiment, the monoclonal antibodies described above with reference to SEQ ID NOs: 9 to 14 (corresponding to “Clone 2”) bind to the danger-associated molecular pattern (DAMPs) OxLDL and DNA. Thus, it is bispecific in terms of the present invention.

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5th edit. NIH Publication no. 91-3242 U.S. Department of Health and Human Services (1991); Chothia J. Mol. Biol. 196 (1987), 901-917 or Chothia Nature 342 (1989), 877-883.

Accordingly, in general terms, an antibody molecule described herein may be selected from the group consisting of a full IgA or IgM or an IgG antibody multivalent F(ab)-, Fab′-SH-, Fv-, Fab′-, F(ab′)2-fragments, a chimeric antibody, a CDR-grafted antibody, a fully human antibody, a bivalent antibody-construct, an antibody-fusion protein, a synthetic antibody, bivalent single chain antibody, a trivalent single chain antibody and a multivalent single chain antibody.

Yet, in a preferred embodiment, the recombinant human monoclonal natural antibody is an IgM and/or IgA antibody.

As already outlined above, “Humanization approaches” are well known in the art and in particular described for antibody molecules, e.g. Ig-derived molecules. The term “humanized” refers to humanized forms of non-human (e.g., murine) antibodies or fragments thereof (such as Fv, Fab, Fab′, F(ab′), scFvs, or other antigen-binding partial sequences of antibodies) which contain some portion of the sequence derived from non-human antibody. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin; see, inter alia, Jones et al., Nature 321 (1986), 522-525, Presta, Curr. Op. Struct. Biol. 2 (1992), 593-596. Methods for humanizing non-human antibodies are well known in the art. Generally, a humanized antibody has one or more amino acids introduced into it from a source which is non-human still retain the original binding activity of the antibody. Methods for humanization of antibodies/antibody molecules are further detailed in Jones et al., Nature 321 (1986), 522-525; Reichmann et al., Nature 332 (1988), 323-327; and Verhoeyen et al., Science 239 (1988), 1534-1536. Specific examples of humanized antibodies, e.g. antibodies directed against EpCAM, are known in the art, see e.g. (LoBuglio, Proceedings of the American Society of Clinical Oncology Abstract (1997), 1562 and Khor, Proceedings of the American Society of Clinical Oncology Abstract (1997), 847).

Accordingly, in the context of this invention, antibody molecules or antigen-binding fragments thereof are provided, which are humanized and can successfully be employed in pharmaceutical compositions.

Moreover, in a preferred embodiment, the recombinant human monoclonal natural IgM and/or IgA antibody recognizing oxidized phospholipids and/or oxidation-specific epitopes for use according to the present invention,
comprises an amino acid sequence with at least 70% sequence identity to the amino acid residues shown in positions 1 to 25, 34 to 50, 59 to 96, and 116 to 126 of SEQ ID NO: 7 and in positions 1 to 25, 35 to 51, 55 to 90, and 101 to 110 of SEQ ID NO: 8, wherein said antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide; or
comprises an amino acid sequence with at least 70% sequence identity to the amino acid residues shown in positions 1 to 25, 34 to 50, 59 to 96, and 116 to 126 of SEQ ID NO: 15 and in positions 1 to 26, 34 to 50, 54 to 89, and 99 to 108 of SEQ ID NO: 16, wherein said antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide.

The monoclonal antibodies which are structurally described above and below with reference to SEQ ID NOs: 7 to 8 and SEQ ID NOs: 15 to 16, respectively, have been characterized in Example 23, further below.

SEQ ID NOs: 7 to 8 are derived from “Clone 1”.

SEQ ID NOs: 15 to 16 are derived from “Clone 2”.

In preferred embodiments, the monoclonal antibodies described above and below with reference to SEQ ID NOs: 7 to 8 and SEQ ID NOs: 15 to 16, respectively, are not monospecific.

In preferred embodiments, the monoclonal antibodies described above and below with reference to SEQ ID NOs: 7 to 8 and SEQ ID NOs: 15 to 16, respectively, are bispecific or multispecific.

Bispecific in this context means that the monoclonal antibody specifically binds to two distinct antigens and/or epitopes of an antigen.

Multispecifc in this context means that the monoclonal antibody specifically binds to more than two distinct antigen and/or epitopes of an antigen, preferably, three, four, or five distinct antigens and/or epitopes of an antigen.

In a more preferred embodiment, said distinct antigen and/or epitope of an antigen is a danger-associated molecular pattern (DAMPs) as already defined herein above.

In a particularly preferred embodiment, said danger-associated molecular pattern (DAMPs) is selected from the group consisting of OxLDL, MDA-LDL, MDA-BSA, PC-BSA and DNA.

Thus, in a particularly preferred embodiment, the monoclonal antibodies described above and below with reference to SEQ ID NOs: 7 to 8 and SEQ ID NOs: 15 to 16, respectively, bind to at least two danger-associated molecular pattern (DAMPs) selected from the group consisting of OxLDL, MDA-LDL, MDA-BSA, PC-BSA and DNA.

In another particularly preferred embodiment, the monoclonal antibodies described above and below with reference to SEQ ID NOs: 7 to 8 (corresponding to “Clone 1”) bind to the danger-associated molecular pattern (DAMPs) OxLDL, MDA-LDL, MDA-BSA, PC-BSA and DNA. Thus, it is multispecific in terms of the present invention.

In another particularly preferred embodiment, the monoclonal antibodies described above and below with reference to SEQ ID NOs: 15 to 16 (corresponding to “Clone 2”) bind to the danger-associated molecular pattern (DAMPs) OxLDL and DNA. Thus, it is bispecific in terms of the present invention.

In a preferred embodiment, the recombinant human monoclonal natural IgM and/or IgA antibody recognizing oxidized phospholipids and/or oxidation-specific epitopes for use according to the present invention comprises or consists of V_{H} domain (heavy chain variable region) and V_{L} domain (light chain variable region),
i.e., the amino acid sequence of the variable region of the heavy chain of an antibody as depicted in SEQ ID NO:7 and the amino acid sequence of the variable region of the light chain of an antibody as depicted in SEQ ID NO:8, wherein said antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide; or
i.e., the amino acid sequence of the variable region of the heavy chain of an antibody as depicted in SEQ ID NO:15 and the amino acid sequence of the variable region of the light chain of an antibody as depicted in SEQ ID NO:16, wherein said antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide.

Assays for determining whether an antibody binds to the above structures are known in the art and can, e.g., be assessed as described herein above.

However, the antibody as used in the present invention is not particularly limited to such variable heavy and light chain variable regions but may also be an antibody or antigen-binding fragment thereof which comprises or consists of V_{H} domain and V_{L} domain with at least 99%, 98%, 97%, 96%, 95%, 90%, 85%, 75%, 70%, 65%, 60%, 55% or 50% sequence identity with the sequences of SEQ ID NOs: 7 and 8, respectively,
the sequences of SEQ ID NOs: 15 and 16, respectively,
as long as the antibody has the capability of recognizing and binding to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide.

Furthermore, the antibody or antigen-binding fragment thereof is a molecule that comprises V_{H} and V_{L} domains having up to 0, 1, 2, 3, 4, 5, 6, 7, 8, 9, 10 or more conservative amino acid substitutions with reference to the sequences of SEQ ID NOs: 7 and 8 or SEQ ID NOs: 15 and 16. Moreover, the antibody or antigen-binding fragment thereof is an antibody fragment selected from the group consisting of Fab, Fab′, Fab′-SH, FV, scFV, F(ab′)2, and a diabody.

In order to determine whether an amino acid sequence has a certain degree of identity to the sequences of SEQ ID NOs: 7, 8, 15 and 16, the skilled person can use means and methods well known in the art, e.g. alignments, either manually or by using computer programs known to the person skilled in the art. Such an alignment can, e.g., be done with means and methods known to the skilled person, e.g. by using a known computer algorithm such as the Lipman-Pearson method (Science 227 (1985), 1435) or the CLUSTAL algorithm. It is preferred that in such an alignment maximum homology is assigned to conserved amino acid residues present in the amino acid sequences. In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity with the nucleic acid sequences or with the amino acid sequences as described above which are capable of binding to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide, when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably, the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

Preferably, the amino acid substitution(s) are “conservative substitution(s)” which refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson Molecular Biology of the Gene, The Benjamin/Cummings Pub. Co. 4th Ed. (1987), 224. In addition, substitutions of structurally or functionally similar amino acids are less likely to disrupt biological activity. Within the context of the present invention the binding compounds/antibodies of the present invention comprise polypeptide chains with sequences that include up to 0 (no changes), 1, 2, 3, 4, 5, 6, 7, 8, 9, 10, 12, 15, 20 or more conservative amino acid substitutions when compared with the specific amino acid sequences disclosed herein, for example, SEQ ID NO: 9 (referring to the variable region of the antibody heavy chain of the antibody) and 10 (referring to the variable of the light chain of the antibody). As used herein, the phrase “up to X” conservative amino acid substitutions includes 0 substitutions and any number of substitutions up to 10 and including 0, 1, 2, 3, 4, 5, 6, 7, 8, 9, 10 substitutions.

Such exemplary substitutions are preferably made in accordance with those set forth in Table 1 as follows:

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Moreover, in a preferred embodiment, the antibody or the antigen-binding fragment thereof for use according to the present invention comprises an amino acid sequence with at least 70% sequence identity to the amino acid residues shown in positions 1 to 25, 34 to 50, 59 to 96, and 116 to 126 of SEQ ID NO: 7 and in positions 1 to 25, 35 to 51, 55 to 90, and 101 to 110 of SEQ ID NO: 8, wherein said antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide.

Moreover, in a preferred embodiment, the antibody or the antigen-binding fragment thereof for use according to the present invention comprises an amino acid sequence with at least 70% sequence identity to the amino acid residues shown in positions 1 to 25, 34 to 50, 59 to 96, and 116 to 126 of SEQ ID NO: 15 and in positions 1 to 26, 34 to 50, 54 to 89, and 99 to 108 of SEQ ID NO: 16, wherein said antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide.

In a further, preferred embodiment, the antibody for use according to the present invention comprises an amino acid sequence with at least 75%, at least 80%, more preferably at least 85%, at least 90%, even more preferably at least 95%, and most preferably 98% overall sequence identity in the framework regions compared to the amino acid residues shown in positions 1 to 25, 34 to 50, 59 to 96, and 116 to 126 of SEQ ID NO: 7 and in positions 1 to 25, 35 to 51, 55 to 90, and 101 to 110 of SEQ ID NO: 8.

Such antibodies are suitable for the medical uses of the present invention as long as the antibody or antigen-binding fragment binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide as described herein above and below.

Thus, in a preferred embodiment, the antibody for use according to the present invention comprises an amino acid sequence having the above variable regions of the light and heavy chains (i.e., the CDRs defined above, i.e., V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6) while the amino acid sequence have a variability in the framework region with at least 75%, at least 80%, more preferably at least 85%, at least 90%, even more preferably at least 95%, and most preferably 98% or even 99 or 100% overall sequence identity in the framework regions compared to the amino acid residues shown in positions 1 to 25, 34 to 50, 59 to 96, and 116 to 126 of SEQ ID NO: 7 and in positions 1 to 25, 35 to 51, 55 to 90, and 101 to 110 of SEQ ID NO: 8.

In a further, preferred embodiment, the antibody for use according to the present invention comprises an amino acid sequence with at least 75%, at least 80%, more preferably at least 85%, at least 90%, even more preferably at least 95%, and most preferably 98% overall sequence identity in the framework regions compared to the amino acid residues shown in positions 1 to 25, 34 to 50, 59 to 96, and 116 to 126 of SEQ ID NO: 15 and in positions 1 to 26, 34 to 50, 54 to 89, and 99 to 108 of SEQ ID NO: 16.

Such antibodies are suitable for the medical uses of the present invention as long as the antibody or antigen-binding fragment binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide as described herein above and below.

Thus, in a preferred embodiment, the antibody for use according to the present invention comprises an amino acid sequence having the above variable regions of the light and heavy chains (i.e., the CDRs defined above, i.e., V_{H}CDR1 comprising SEQ ID NO: 9, V_{H}CDR2 comprising SEQ ID NO: 10, V_{H}CDR3 comprising SEQ ID NO: 11, V_{L}CDR1 comprising SEQ ID NO: 12, V_{L}CDR2 comprising SEQ ID NO: 13, and V_{L}CDR3 comprising SEQ ID NO:14) while the amino acid sequence have a variability in the framework region with at least 75%, at least 80%, more preferably at least 85%, at least 90%, even more preferably at least 95%, and most preferably 98% or even 99 or 100% overall sequence identity in the framework regions compared to the amino acid residues shown in positions 1 to 25, 34 to 50, 59 to 96, and 116 to 126 of SEQ ID NO: 15 and in positions 1 to 26, 34 to 50, 54 to 89, and 99 to 108 of SEQ ID NO: 16.

In this context, a polypeptide has “at least X % sequence identity” in the framework regions to SEQ ID NO:7 or 8 (or SEQ ID NO: 15 or 16) if SEQ ID NO:7 or SEQ ID NO: 8 (or SEQ ID NO: 15 or 16) is aligned with the best matching sequence of a polypeptide of interest and the amino acid identity between those two aligned sequences is at least X % over positions 1 to 25, 34 to 50, 59 to 96, and 116 to 126 of SEQ ID NO: 7 (or SEQ ID NO: 15) and in positions 1 to 25, 35 to 51, 55 to 90, and 101 to 110 of SEQ ID NO: 8 (or SEQ ID NO: 16). As mentioned above, such an alignment of amino acid sequences can be performed using, for example, publicly available computer homology programs such as the “BLAST” program provided on the National Centre for Biotechnology Information (NCBI) homepage using default settings provided therein. Further methods of calculating sequence identity percentages of sets of amino acid sequences or nucleic acid sequences are known in the art.

Moreover, in a preferred embodiment, the antibody or the antigen-binding fragment thereof for use according to the present invention comprises the V_{H} of SEQ ID NO:7 and the V_{L} of SEQ ID NO:8.

Moreover, in a preferred embodiment, the antibody or the antigen-binding fragment thereof for use according to the present invention comprises the V_{H} of SEQ ID NO:7 and the V_{L} of SEQ ID NO:8, wherein said antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide.

Moreover, in a preferred embodiment, the antibody or the antigen-binding fragment thereof for use according to the present invention comprises the V_{H} of SEQ ID NO:15 and the V_{L} of SEQ ID NO:16.

Moreover, in a preferred embodiment, the antibody or the antigen-binding fragment thereof for use according to the present invention comprises the V_{H} of SEQ ID NO:15 and the V_{L} of SEQ ID NO:15, wherein said antibody recognizes and binds to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide.

The term “recognizing and binding to” a certain structure has been described above which applies, mutatis mutandis, to the above-described antibodies which “recognize and bind phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide”.

In a preferred embodiment, the antibody which “recognize and bind to phosphorylcholine exposed by oxidized phosphatidylcholine and/or oxidized 1-palmitoyl-2-arachidonoyl-phosphatidylcholine, to oxidized cardiolipin, to oxidized phosphatidylserine, to malondialdehyde-, 4-hydroxynonenal- and 2-(ω-carboxyethyl)-pyrrole-modified proteins, and/or to oligomeric amyloid-β peptide” has a dissociation constant KD of at most 0.5×10⁻³ nM, at most 1.0×10⁻³ M, at most 0.5×10⁻⁴M, at most 1.0×10⁻⁴ M, at most 5×10⁻⁴ M, at most 1×10⁻⁵ M, at most 3×10⁻⁵ M, at most 5.0×10⁻⁵ M, at most 1.0×10⁻⁶ M, preferably at most 0.5×10⁻⁷ M, more preferably at most 1.0×10⁻⁷ M, even more preferably at most 1.0×10⁻⁸ M, and most preferably at most 1.0×10⁻⁹ M. The KD represents the dissociation constant as a measure of the propensity of a complex to dissociate reversibly into its components (i.e. the affinity of the antibody for the antigen) and is the inverse of the association constant.

The above values relate to the binding per binding site of the antibody.

The KD may be calculated from the Scatchard equation and methods for determining KD are well known in the art.

In the present case, as the antibody preferably concerns an IgM antibody consisting of five antibodies (and, accordingly, is present in the form of a pentamer has ten binding sites), the avidity is rather high and has a dissociation constant KD of at most 0.5×10⁻³ nM, at most 1.0×10⁻³ M, at most 0.5×10⁻⁴M, at most 1.0×10⁻⁴ M, at most 5×10⁻⁴ M, at most 1×10⁻⁵ M, at most 3×10⁻⁵ M, at most 5.0×10⁻⁵ M, at most 1.0×10⁻⁶ M, preferably at most 0.5×10⁻⁷ M, more preferably at most 1.0×10⁻⁷ M, even more preferably at most 1.0×10⁻⁸ M, and most preferably at most 1.0×10⁻⁹ M.

Avidity is a measure of the accumulated strength of multiple affinities of individual non-covalent binding interactions, such as between an antibody and its antigen. As such, avidity is distinct from affinity, which describes the strength of a single interaction.

The recombinant human or humanized natural IgM and/or IgA antibody recognizing oxidized phospholipids and/or oxidation-specific epitopes for use according to the present invention is not limited to the above specific antibody/antibodies but may be any recombinant human monoclonal natural IgM antibody as long as it recognizes and binds to oxidized phospholipids and/or oxidation-specific epitopes.

With the normal skill of the person skilled in the art and by routine methods, the person skilled in the art equipped with this description of the present invention can easily deduce from the structure of oxidized phospholipids and/or oxidation-specific epitopes relevant epitopes (also functional fragments) which are useful in the generation of antibodies like polyclonal and monoclonal antibodies. However, the person skilled in the art is readily in a position to also provide for engineered antibodies like CDR-grafted antibodies or also humanized and fully human antibodies and the like.

As mentioned above, particularly preferred in the context of the present invention are monoclonal antibodies. For the preparation of monoclonal antibodies, any technique which provides antibodies produced by continuous cell line cultures can be used. Examples for such techniques include the hybridoma technique, the trioma technique, the human B-cell hybridoma technique and the EBV-hybridoma technique to produce human monoclonal antibodies (Shepherd and Dean (2000), Monoclonal Antibodies: A Practical Approach, Oxford University Press, Goding and Goding (1996), Monoclonal Antibodies: Principles and Practice—Production and Application of Monoclonal Antibodies in Cell Biology, Biochemistry and Immunology, Academic Pr Inc, USA).

The antibody (derivatives) can also be produced by peptidomimetics. Further, techniques described for the production of single chain antibodies (see, inter alia, U.S. Pat. No. 4,946,778) can be adapted to produce single chain antibodies specifically recognizing an antigen. Also, transgenic animals may be used to express humanized antibodies to the desired antigen.

The present invention also envisages the production of specific antibodies against oxidized phospholipids and/or oxidation-specific epitopes. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

As a second example, this production is based on the single-cell sorting of human B cells and expression-cloning of the immunoglobulin heavy and light chain genes thereof. In this example, single human B cells can be sorted based on the expression of specific surface markers, such as CD20^{pos}CD27^{pos}CD43^{pos}CD70^{neg} to identify B1 cells, or based on binding of fluorescently labelled specific antigens such as phosphorylcholine or MDA adducts. As a third example, this production is based on the sorting of human B cells based on the expression of specific surface markers, such as CD20^{pos}CD27^{pos}CD43^{pos}CD70^{neg} to identify B1 cells, or based on binding of fluorescently labelled specific antigens such as phosphorylcholine or MDA adducts. The sorted B cells can be transduced with EBV virus to immortalize the B cells, followed by single-cell cloning using methods such as single-cell FACS-sorting or limiting dilution. The supernatant obtained from thereby generated B cell lines can be tested for antigen-specificity and the immunoglobulin heavy and light chain genes can be cloned from B cell clones expressing the desired antibody specificity. As a fourth example, this production is based on selecting antibodies out of combinatorial antibody-phage display libraries. In this example, antigens such as phosphorylcholine or MDA adducts can be used to select specific antibodies.

The term “specifically binds”, as used herein, refers to a binding reaction that is determinative of the presence of the desired oxidized phospholipids and/or oxidation-specific epitopes, and an antibody in the presence of a heterogeneous population of proteins and other biologics.

Thus, under designated assay conditions, the specified antibodies and a corresponding oxidized phospholipid and/or oxidation-specific epitope, bind to one another and do not bind in a significant amount to other components present in a sample.

Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell (2000) Basic Methods in Antibody Production and Characterization, Crc. Pr. Inc. for a description of immunoassay formats and conditions that can be used to determine specific immunoreactivity. Typically a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. cit.).

The term “antibody or antigen-binding fragment thereof” means in accordance with this invention that the antibody molecule or antigen-binding fragment thereof is capable of specifically recognizing or specifically interacting with and/or binding to at least a partial structure of said oxidized phospholipid and/or oxidation-specific epitope. Said term relates to the specificity of the antibody molecule, i.e. to its ability to discriminate between the specific regions of an oxidized phospholipid and/or oxidation-specific epitope. Accordingly, specificity can be determined experimentally by methods known in the art and methods as disclosed and described herein. Such methods comprise, but are not limited to Western blots, ELISA-, RIA-, ECL-, IRMA-tests and peptide scans. Such methods also comprise the determination of K_{D}-values known in the art.

In a preferred embodiment, regarding the recombinant human monoclonal natural IgM and/or IgA antibody recognizing oxidized phospholipids and/or oxidation-specific epitopes for use according to present invention in accordance with the second aspect of the present invention, said disorder or a disease associated with/related to/caused by natural IgM/IgA antibody deficiency (NAD) is a natural antibody infectious, neurodegenerate, metabolic, autoimmune or cardiovascular disease.

As regards said natural antibody deficient infectious, neurodegenerate, metabolic, autoimmune or cardiovascular disease, the same applies, mutatis mutandis, as has been set forth above in the context of the first aspect of the present invention.

In fact, as regards autoimmune diseases, it has surprisingly been found in the present invention (see Example 22 and FIG. 3) that in patients with severe COVID-19, autoimmune IgG antibodies are generated. These data support that the lack of natural antibodies (nABs) in terms of the present invention can result in the development of autoimmune antibodies during severe COVID-19 courses. The presence of these autoimmune antibodies provides evidence for recurring or long-lasting COVID-19 disease symptoms, supporting that sufficient levels of natural antibodies, provision of (monoclonal) natural IgMs or IgAs, or preparations enriched for natural antibodies (e.g. Pentaglobin®) in terms of the present invention can prevent the generation or reduce the levels of autoimmune antibodies.

In a preferred embodiment, regarding the recombinant human monoclonal natural IgM and/or IgA antibody recognizing oxidized phospholipids and/or oxidation-specific epitopes for use according to present invention in accordance with the second aspect of the present invention, said disorder or a disease associated with/related to/caused by natural IgM/IgA antibody deficiency (NAD) is the virus infection disease COVID-19 caused by the β-Coronavirus SARS-CoV-2.

In another preferred embodiment, regarding the recombinant human monoclonal natural IgM and/or IgA antibody recognizing oxidized phospholipids and/or oxidation-specific epitopes for use according to present invention in accordance with the second aspect of the present invention, said disorder or a disease associated with/related to/caused by natural IgM/IgA antibody deficiency (NAD) is long COVID-19.

As regards said virus infection disease COVID-19 caused by the β-Coronavirus SARS-CoV-2 and long COVID-19, respectively, the same applies, mutatis mutandis, as has been set forth above in the context of the first aspect of the present invention.

In a preferred embodiment, regarding the recombinant human monoclonal natural IgM and/or IgA antibody recognizing oxidized phospholipids and/or oxidation-specific epitopes for use according to present invention in accordance with the second aspect of the present invention, said antibody is capable of virus neutralization and/or inhibiting the spreading of a virus from an infected cell to an adjacent second non-infected cell (cell-to-cell spread).

As regards said capability of inhibiting the spreading of a virus from an infected cell to an adjacent second non-infected cell (cell-to-cell spread), the same applies, mutatis mutandis, as has been set forth above in the context of the first aspect of the present invention.

In a preferred embodiment, regarding the recombinant human monoclonal natural IgM and/or IgA antibody recognizing oxidized phospholipids and/or oxidation-specific epitopes for use according to present invention in accordance with the second aspect of the present invention, said antibody is capable of neutralizing the infection by a virus and to, thereby, preventing the infection of target cells.

As regards said capability of “virus neutralization” and/or “neutralizing the infection by a virus and to, thereby, preventing the infection of target cells”, the same applies, mutatis mutandis, as has been set forth above in the context of the first aspect of the present invention.

In a preferred embodiment, regarding the recombinant human monoclonal natural IgM and/or IgA antibody recognizing oxidized phospholipids and/or oxidation-specific epitopes for use according to present invention in accordance with the second aspect of the present invention, said antibody has an anti-inflammatory activity, preferably the capability of:
reducing the accumulation of free oxidized phospholipids, preferably in infect lungs, clearing cellular debris in lung tissue, and/or
stimulating IL-10 and/or TGFβ secretion;

As regards said anti-inflammatory activity, preferably said capability of: reducing the accumulation of free oxidized phospholipids, preferably in infect lungs, clearing cellular debris in lung tissue, and/or stimulating IL-10 and/or TGFβ secretion; and/or neutralizing of pro-inflammatory cytokines the same applies, mutatis mutandis, as has been set forth above in the context of the first aspect of the present invention.

In a preferred embodiment, regarding the recombinant human monoclonal natural IgM and/or IgA antibody recognizing oxidized phospholipids and/or oxidation-specific epitopes for use according to present invention in accordance with the second aspect of the present invention, said disorder or disease associated with natural IgM/IgA antibody deficiency (NAD) is an inflammatory disease or a virus infection disease.

As regards said disorder or a disease associated with natural IgM/IgA antibody deficiency (NAD) is an inflammatory disease or a virus infection disease, the same applies, mutatis mutandis, as has been set forth above in the context of the first aspect of the present invention.

In a preferred embodiment, regarding the recombinant human monoclonal natural IgM and/or IgA antibody recognizing oxidized phospholipids and/or oxidation-specific epitopes for use according to present invention in accordance with the second aspect of the present invention, said inflammatory disease is selected from the group consisting of wherein said inflammatory disease is selected from the group consisting of infectious diseases mediated by respiratory viruses, preferably COVID19, influenza, MERS-COV or SARS-COV; infectious diseases caused by bacterial infections mediated by gram positive or gram negative pathogens, fungi, or parasites; and sterile diseases, preferably cardiovascular diseases, atherosclerosis, coronary heart disease, heart attack and stroke, metabolic disorders like diabetes mellitus, neurodegenerative diseases, preferably Alzheimer's Disease, and autoimmune diseases, preferably Systemic Lupus Erythematodes, or Multiple Sclerosis.

As regards said inflammatory disease being selected from the group consisting of infectious diseases mediated by respiratory viruses, preferably COVID19, influenza, MERS-COV or SARS-COV; infectious diseases caused by bacterial infections mediated by gram positive or gram negative pathogens, fungi, or parasites; and sterile diseases, preferably cardiovascular diseases, atherosclerosis, coronary heart disease, heart attack and stroke, metabolic disorders like diabetes mellitus, neurodegenerative diseases, preferably Alzheimer's Disease, and autoimmune diseases, preferably Systemic Lupus Erythematodes, or Multiple Sclerosis., the same applies, mutatis mutandis, as has been set forth above in the context of the first aspect of the present invention.

In a preferred embodiment, regarding the recombinant human monoclonal natural IgM and/or IgA antibody recognizing oxidized phospholipids and/or oxidation-specific epitopes for use according to present invention in accordance with the second aspect of the present invention, said virus infection disease is selected from the group consisting of infections by coronaviruses, preferably SARS-CoV, SARS-CoV-2, MERS); influenza viruses, parainfluenza viruses, respiratory syncytial viruses (RSV), rhinoviruses, adenoviruses, enteroviruses, human metapneumoviruses, herpesviruses, preferably HSV-1, HSV-2, VZV, EBV, HCMV, HHV-6, HHV-7, HHV-8. As regards said virus infection disease being selected from the group consisting of infections by coronaviruses, preferably SARS-CoV, SARS-CoV-2, MERS); influenza viruses, parainfluenza viruses, respiratory syncytial viruses (RSV), rhinoviruses, adenoviruses, enteroviruses, human metapneumoviruses, herpesviruses, preferably HSV-1, HSV-2, VZV, EBV, HCMV, HHV-6, HHV-7, HHV-8, the same applies, mutatis mutandis, as has been set forth above in the context of the first aspect of the present invention.

The antibodies as defined above are particularly useful in medical settings.

Thus, in a preferred embodiment, the present invention relates to a pharmaceutical composition, comprising an effective amount of the recombinant human monoclonal natural IgM and/or IgA antibody recognizing oxidized phospholipids and/or oxidation-specific epitopes for use according to the present invention as described above and at least one pharmaceutically acceptable excipient.

The term “treatment” and/or “prevention” and the like are used herein to generally mean obtaining a desired pharmacological and/or physiological effect. Accordingly, the treatment of the present invention may relate to the treatment of (acute) states of a certain disease but may also relate to the prophylactic treatment in terms of completely or partially preventing a disease or symptom thereof. Preferably, the term “treatment” is to be understood as being therapeutic in terms of partially or completely curing a disease and/or adverse effect and/or symptoms attributed to the disease. “Acute” in this respect means that the subject shows symptoms of the disease. In other words, the subject to be treated is in actual need of a treatment and the term “acute treatment” in the context of the present invention relates to the measures taken to actually treat the disease after the onset of the disease or the outbreak of the disease. The treatment may also be prophylactic or preventive treatment, i.e., measures taken for disease prevention, e.g., in order to prevent the infection and/or the onset of the disease.

The pharmaceutical composition of the present invention may be administered via a large range of classes of forms of administration known to the skilled person. Administration may be systemically, locally, orally, through aerosols including but not limited to tablets, needle injection, the use of inhalators, creams, foams, gels, lotions and ointments.

An excipient or carrier is an inactive substance formulated alongside the active ingredient, i.e., the antibody as described above of the present invention for the purpose of bulking-up formulations that contain potent active ingredients. Excipients are often referred to as “bulking agents,” “fillers,” or “diluents”. Bulking up allows convenient and accurate dispensation of a drug substance when producing a dosage form. They also can serve various therapeutic-enhancing purposes, such as facilitating drug absorption or solubility, or other pharmacokinetic considerations. Excipients can also be useful in the manufacturing process, to aid in the handling of the active substance concerned such as by facilitating powder flowability or non-stick properties, in addition to aiding in vitro stability such as prevention of denaturation over the expected shelf life. The selection of appropriate excipients also depends upon the route of administration and the dosage form, as well as the active ingredient and other factors.

Thus, the pharmaceutical composition comprising an effective amount of the antibody of the present invention as described above may be in solid, liquid or gaseous form and may be, inter alia, in a form of (a) powder(s), (a) tablet(s), (a) solution(s) or (an) aerosol(s). It is preferred that said pharmaceutical composition optionally comprises a pharmaceutically acceptable carrier and/or diluent.

These pharmaceutical compositions can be administered to the subject at a suitable dose. Administration of the suitable compositions may be affected by different ways, e.g., by intravenous, intraperitoneal, subcutaneous, intramuscular, topical, intradermal, intranasal or intrabronchial administration. It is particularly preferred that said administration is carried out by injection and/or delivery, e.g., to a site in a lung artery or directly into the lung. The compositions of the invention may also be administered directly to the target site, e.g., by biolistic delivery to an external or internal target site, like the lung. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg/kg body weight per dose; however, doses below or above this exemplary range are envisioned, especially considering the aforementioned factors. If the regimen is a continuous infusion, it should also be in the range of 1 μg to 10 mg units per kilogram of body weight per minute.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose, i.e., in “an effective amount” which can easily be determined by the skilled person by methods known in the art. The dosage regimen will be determined by the attending physician and clinical factors. As is well known in the medical arts, dosages for any one patient depends upon many factors, including the patient's or subject's size, body surface area, age, the particular compound to be administered, sex, time and route of administration, general health, and other drugs being administered concurrently.

Thus, preferably, the antibody of the present invention as described above is included in an effective amount. The term “effective amount” refers to an amount sufficient to induce a detectable therapeutic response in the subject to which the pharmaceutical composition is to be administered. In accordance with the above, the content of the antibody of the present invention in the pharmaceutical composition is not limited as far as it is useful for treatment as described above, but preferably contains 0.0000001-10% by weight per total composition. Further, the antibody described herein is preferably employed in a carrier. Generally, an appropriate amount of a pharmaceutically acceptable salt is used in the carrier to render the composition isotonic. Examples of the carrier include but are not limited to saline, Ringer's solution and dextrose solution. Preferably, acceptable excipients, carriers, or stabilisers are non-toxic at the dosages and concentrations employed, including buffers such as citrate, phosphate, and other organic acids; salt-forming counter-ions, e.g. sodium and potassium; low molecular weight (>10 amino acid residues) polypeptides; proteins, e.g. serum albumin, or gelatine; hydrophilic polymers, e.g. polyvinylpyrrolidone; amino acids such as histidine, glutamine, lysine, asparagine, arginine, or glycine; carbohydrates including glucose, mannose, or dextrins; monosaccharides; disaccharides; other sugars, e.g. sucrose, mannitol, trehalose or sorbitol; chelating agents, e.g. EDTA; non-ionic surfactants, e.g. Tween, Pluronics or polyethylene glycol; antioxidants including methionine, ascorbic acid and tocopherol; and/or preservatives, e.g. octadecyldimethylbenzyl ammonium chloride; hexamethonium chloride; benzalkonium chloride, benzethonium chloride; phenol, butyl or benzyl alcohol; alkyl parabens, e.g. methyl or propyl paraben; catechol; resorcinol; cyclohexanol; 3-pentanol; and m-cresol). Suitable carriers and their formulations are described in greater detail in Remington's Pharmaceutical Sciences, 17th ed., 1985, Mack Publishing Co.

Therapeutic progress can be monitored by periodic assessment. The antibody of the present invention or the pharmaceutical composition of the invention may be in sterile aqueous or non-aqueous solutions, suspensions, and emulsions as well as creams and suppositories. Examples of non-aqueous solvents are propylene glycol, polyethylene glycol, vegetable oils such as olive oil, and organic esters such as ethyl oleate. Aqueous carriers include water, alcoholic/aqueous solutions, emulsions or suspensions, including saline and buffered media. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition. Said agents may be, e.g., polyoxyethylene sorbitan monolaurate, available on the market with the commercial name Tween, propylene glycol, EDTA, Citrate, Sucrose as well as other agents being suitable for the intended use of the pharmaceutical composition that are well-known to the person skilled in the art.

In accordance with this invention, the term “pharmaceutical composition” relates to a composition for administration to a patient, preferably a human patient.

The invention also relates to method of treating or preventing a disorder or a disease associated with/related to/caused by a natural IgM/IgA antibody deficiency (NAD) in a subject as defined herein above.

As regards the preferred embodiments of the method for treatment the same applies, mutatis mutandis, as has been set forth above in the context of the antibody or the pharmaceutical composition for use as defined above.

In the present invention, the subject is, in a preferred embodiment, a mammal such as a dog, cat, pig, cow, sheep, horse, rodent, e.g., rat, mouse, and guinea pig, or a primate, e.g., gorilla, chimpanzee, and human. In a most preferable embodiment, the subject is a human.

In the present invention, in a preferred embodiment of the first and second aspect as defined above,
said human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject is to be administered in combination with an immunomodulator. Preferably, such a combination therapy exerts synergistic effects on the treatment in accordance with the present invention.

As regards the preferred embodiments of such a combination therapy, the same applies, mutatis mutandis, as has been set forth above in the context of the first and/or second aspect of the present invention and/or the antibody or the pharmaceutical composition for use as defined above.

The term “combination” as used herein relates to a combination of a human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject as described herein above and an immunomodulator described herein below.

In a preferred embodiment, a simultaneous application is envisaged. Yet, the combination also encompasses a subsequent application of the two components, i.e. an human or humanized natural IgM and/or IgA antibody recognizing oxidized phospholipids and/or oxidation-specific epitopes as outlined above and an immunomodulator described herein below. Thus, one of these components may be administered before, simultaneously with or after the other one of the combination, or vice versa.

Accordingly, “in combination” as used herein does not restrict the timing between the administration of the human or humanized natural IgM and/or IgA antibody recognizing oxidized phospholipids and/or oxidation-specific epitopes as outlined above and an immunomodulator described herein below. Thus, when the two components are not administered simultaneously with/concurrently, the administrations may be separated by 1 minute, 5 minutes, 15 minutes, 30 minutes, 45 minutes, 1 hour, 2 hours, 4 hours, 6 hours, 12 hours, 24 hours, 48 hours or 72 hours or by any suitable time differential readily determined by one of skill in art and/or described herein. In a preferred embodiment, when the two components are not administered simultaneously with/concurrently, the administrations may be separated by 1 hour, 2 hours, 4 hours, 6 hours, 12 hours, 24 hours, 48 hours or 72 hours or by any suitable time differential readily determined by one of skill in art and/or described herein.

Immunomodulators are well-known to the person skilled in the art and are commonly also referred to an agent or drug that has the capability to modulate the behaviour and/or function of specific immune cells or other cell types such as endothelial cells of the host.

It is known that immunomodulators act at different levels of the immune system. Therefore different kinds of agents or drugs have been developed that selectively either inhibit or intensify/enhance the specific populations and subpopulations of immune responsive cells, i.e., lymphocytes, macrophages, neutrophils, natural killer (NK) cells, and cytotoxic T lymphocytes (CTL).

More specifically, in preferred embodiments, such an immunomodulator, immunomodulatory agent or drug can have immunostimulatory (proinflammatory) effects on an immune response. Thus, in preferred embodiments, the immunomodulator, immunomodulatory agent or drug can be immunostimulator.

Alternatively, in other preferred embodiments, such an immunomodulator, immunomodulatory agent or drug can have immunosuppressive (anti-inflammatory) effects on an immune response.

Immunomodulators are well-known to the person skilled in the art and an immunomodulator in accordance with the present invention is not limited to specific immunomodulators. Rather, the skilled person is in a position to select a suitable immunomodulator as long as it has the above functional capabilities.

In preferred embodiments, immunomodulatory drugs can either be small molecules or biologics. The nature of the immunomodulatory drug being a small molecule or a biologics is not limited. In preferred embodiments, the immunomodulatory drugs can be selected from the group consisting of anti-PD-1, anti-PD-L1, anti-CD40 (Agonist), CD40-Ligand, anti-GM-CSF, anti-CSF-1R, anti-CTLA-4, anti-IL-6, anti-IL-6R, anti-CCL2, anti-CCL5, anti-CCR5 (Antagonist), anti-CCR2 (Antagonist). Examples of immunomodulatory agents are, but are not limited to, antibodies that bind to cytokines such as IL-6, or to its specific receptor such as the IL-6R, and thereby neutralize the proinflammatory effects of the cytokine on other cell types including immune cells.

Other examples of immunomodulatory agents are, but are not limited to, antibodies that bind to chemokines such as CCL2 and CCL5, or to their specific receptors such as CCR2 and CCR5, and thereby neutralize the chemotactic effects of the chemokines on other cell types including immune cells.

Yet other examples of immunomodulatory agents are, but are not limited to, synthetic molecules such as Maraviroc that bind to receptors of chemokines such as CCR5, and thereby neutralize the chemotactic effects of the chemokine on other cell types including immune cells.

The skilled person is in a position to select an appropriate immunomodulator that is suitable to modulate the behaviour and/or function of specific immune cells or other cell types such as endothelial cells in accordance with the present invention and in accordance with the above.

As examples, immunomodulators may be selected from the group consisting of antibodies that bind to cytokines (preferably to IL-6), antibodies that bind to its specific cytokine receptor (preferably to IL-6R), antibodies that bind to chemokine(s) (preferably to CCL2 and CCL5), antibodies that bind to their specific chemokine receptor(s) (preferably to CCR2 and CCR5), synthetic molecules (preferably Maraviroc).

In a more preferred embodiment, the immunomodulator is Maraviroc.

Maraviroc belongs to the CCR5 receptor antagonist class and is used as an antiretroviral drug in the treatment of HIV infection. It is also classed as an entry inhibitor. It also reduces graft-versus-host disease in patients treated with allogeneic bone marrow transplantation for leukemia. Maraviroc is an entry inhibitor. Specifically, Maraviroc is a negative allosteric modulator of the CCR5 receptor, which is found on the surface of certain human cells. The chemokine receptor CCR5 is an essential co-receptor for most HIV strains and necessary for the entry process of the virus into the host cell. The drug binds to CCR5, thereby blocking the HIV protein gp120 from associating with the receptor. HIV is then unable to enter human macrophages and T cells.

In another, also more preferred embodiment, the immunomodulator is dexamethason. Dexamethason is a low-cost corticosteroid medication that is used in a variety of inflammatory diseases, since it has anti-inflammatory and immunosuppressant effects. Thus, dexamethason is an anti-inflammatory and immunosuppressant immunomodulator. The preliminary report of the RECOVERY trial conducted by the University of Oxford (UK) showed that use of dexamethasone reduced the incidence of death by approximately one-third in ventilated patients with severe COVID-19. This is likely due to its anti-inflammatory and immunosuppressive effects which are most prominent in patients with severe disease, since they show strong pro-inflammatory profiles. In contrast, in COVID-19 patients not requiring ventilation dexamethasone had no beneficial effect, in contrary may even have caused an increase in the incidence of deaths. Dexamethasone treatment has been shown to induce expression of ACE, which causes the production of angiotensin II. In addition, angiotensin II type 1 receptor expression was also demonstrated to be increased by dexamethasone, hence dexamethasone likely contributes to pro-inflammatory signaling through angiotensin II and to increased oxidative stress and accumulation of oxidized phospholipids/OSE. While in severe COVID-19 cases the immunosuppressive and anti-inflammatory effects of dexamethasone likely outweigh this pro-inflammatory effect, in milder COVID-19 cases, in which patients do not require ventilation, the effects of dexamethasone-stimulated angiotensin II-induced oxidative stress may become visible, reflected in the observed increase of incidences of deaths.

Preferably, a combination therapy of dexamethasone with oxidized phospholipid-/OSE-specific natural IgM/IgA antibodies in terms of the present invention exerts synergistic effects and enhance the efficacy of treatment even in patients with milder COVID-19 cases that do not require ventilation.

In the present invention, in a preferred embodiment of the first and second aspect as defined above,
said human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject is to be administered in combination with an antiviral compound, preferably wherein said antiviral compound is:
remdesivir;
favipiravir;
camostat mesylate;
nafamostat mesylate;
umifenovir; and/or
stronger neo-minophagen C.

Preferably, such a combination therapy exerts synergistic effects on the treatment in accordance with the present invention.

As regards the preferred embodiments of such a combination therapy, the same applies, mutatis mutandis, as has been set forth above in the context of the first and/or second aspect of the present invention and/or the antibody or the pharmaceutical composition for use as defined above.

As regards the term “combination” and “combination therapy” regarding the combination with an antiviral agent, preferrably with remdesivir; favipiravir; camostat mesylate; nafamostat mesylate; umifenovir; and/or stronger neo-minophagen C and the preferred embodiments of such a combination, the same applies, mutatis mutandis, as has been set forth above in the context of the combination therapy with an immunomodulator.

Indeed, as outlined above, the present invention is based on the common feature of host immune failure that in a variety of infectious diseases massive formation of oxidized phospholipids and OSE accumulate in the lung of also SARS-CoV-2 infected patients that trigger pro-inflammatory cytokine production in macrophages and thereby initiate the deterioration phase in COVID-19 (and other infectious diseases). In terms of the present invention, high levels of circulating OSE-specific IgM and possibly IgA antibodies confer protection because they bind to oxidized phospholipids and OSE and thereby promote their save clearance, which in turn counteracts the induction of fatal cytokine storm syndrome and ARDS.

Hence, as mentioned above, it has surprisingly been described in the present application that a subgroup of natural IgM and/or IgA antibodies can be used in the treatment or prevention of a disorder or a disease associated with/related to/caused by a natural IgM/IgA antibody deficiency (NAD) in a subject, i.e., human or humanized natural IgM and/or IgA antibody recognizing oxidized phospholipids and/or oxidation-specific epitopes. As explained above, the present invention is based on the surprising observation that the stimulatory effects of oxidized phospholipids (oxPL) and oxidation-specific epitopes (OSE) on macrophage activation and secretion of pro-inflammatory cytokines such as IL-6 contributes to the initiation of fatal cytokine release syndrome and the development of acute lung injury and ARDS in severe COVID-19 patients.

This observation leads to the first and second aspect of the present invention as defined above that a human or humanized natural IgM and/or IgA antibody recognizing oxidized phospholipids and/or oxidation-specific epitopes can be used in a method of treating or preventing a disorder or a disease associated with/related to/caused by a natural IgM/IgA antibody deficiency (NAD) in a subject.

Once these embodiments of the present invention found that the reduction of oxidized phospholipids and/or oxidation-specific epitopes by the use of human or a humanized natural IgM and/or IgA antibody recognizing oxidized phospholipids and/or oxidation-specific epitopes can be used to treat or prevent a disorder or a disease associated with/related to/caused by a natural IgM/IgA antibody deficiency (NAD) in a subject, without being bound by theory, this surprising observation opens for the possibility for a combinational therapy of compounds stimulating the generation of natural IgM/IgA antibodies, or comprising natural IgM/IgA antibodies (e.g. natural IgM/IgA antibody-enriched plasma or serum preparations), or comprising recombinant monoclonal oxidized phospholipid- or OSE-specific natural IgM/IgA antibodies with compounds acting directly against the pathogen, in the case of SARS-CoV-2 in particular, but not exclusively, the antiviral compounds, preferrably remdesivir, favipiravir (Avigan), camostat mesylate, nafamostat mesylate, umifenovir (Arbidol) and stronger neo-minophagen C (SNMC), generate a synergistic therapeutic effect through 1) the elimination of the source of increased oxidized phospholipid/OSE production, i.e. virus replication and 2) the reduction of accumulated oxidized phospholipid/OSE products via the oxidized phospholipid-/OSE-specific natural IgM/IgA antibodies.

Antiviral compounds are well-known to the person skilled in the art and are commonly also referred to an agent or drug or compound that has the capability to inhibit the development and/or propagation of viruses. Antiviral compounds, accordingly, refer to an agent or drug or compound used to treat a viral infection. Most antivirals target specific viruses, while a broad-spectrum antiviral is effective against a wide range of viruses. Unlike most antibiotics, antiviral drugs do not destroy their target pathogen; instead they inhibit their development. Thus, antiviral compounds in terms of the present invention relates to an agent that kills a virus and/or that suppresses the virus' ability to replicate, thereby inhibiting the capability of the virus to multiply and/or reproduce.

Antiviral compounds are well-known to the person skilled in the art and an antiviral compound in accordance with the present invention is not limited to specific antiviral compound. Rather, the skilled person is in a position to select a suitable antiviral compound as long as it has the above functional capabilities.

With respect to a method of treating or preventing a disorder or a disease associated with/related to/caused by a natural IgM/IgA antibody deficiency (NAD) in a subject in terms of the present invention, the skilled person is in a position to select an appropriate antiviral compound that is suitable to inhibit the virus' development in accordance with the present invention. As examples, an antiviral compound may be selected from the group consisting of the drug classes of direct-acting antiviral compounds and indirect-acting antiviral compounds.

In fact, it is known to the skilled person that antiviral compounds are generally divided into two main classes based on their way of action, namely said direct-acting antiviral compounds and indirect-acting antiviral compounds.

Direct-acting antiviral compounds, based on their stage of effect during the viral replication, are further sub-divided into entry-inhibitors, protease-inhibitors, replikase-inhibitors, inhibitors of virus production, inhibitors of virus release and inhibitors of virus maturation.

Indirect-acting antiviral compounds exert their function by acting on cellular factors that modulate the cell so that the replication of the virus is inhibited.

In preferred embodiments, the antiviral compound is selected from the group consisting of remdesivir; favipiravir; camostat mesylate; nafamostat mesylate; umifenovir; and stronger neo-minophagen C (SNMC).

The above remdesivir und favipiravir are classified as direct-acting antiviral compounds (acting on the replication of viruses), camostat mesylate, nafamostat mesylate und stronger neo-minophagen C (SNMC) are classified as indirect-acting antiviral compounds while umifenovir has a dual function.

Remdesivir (also known as GS-5734) is a monophosphoramidate prodrug of an adenosine analogue that is a direct-acting antiviral and has a broad antiviral spectrum including filoviruses, paramyxoviruses, pneumoviruses, and coronaviruses. Remdesivir inhibits virus replication by blocking the RNA-dependent RNA polymerase causing premature delayed chain-termination during viral RNA-synthesis. It has been investigated in clinical trials as a therapy against Ebola virus infection, was well tolerated, however, was less effective than monoclonal antibody therapies. Remdesivir is a potent inhibitor of SARS-CoV-2 replication in vitro and has shown clinical benefits in rhesus macaques infected with SARS-CoV-2. In the clinical phase 3 SIMPLE trial in patients with moderate COVID-19, patients treated for 5 days with remdesivir were 65% more likely to have clinical improvement compared to the standard of care group. Clinical improvement was also shown in the National Institute of Allergy and Infectious Diseases (NIAID) ACTT-1 trial in hospitalized patients with a range of disease severities. Remdesivir was approved under an Emergency Use Authorization (EUA) for treatment of COVID-19 patients.

Favipiravir (Avigan) is a prodrug that is metabolized to the antiviral product favipiravir-ribofuranosyl-5′-triphosphate and in a similar mode of action as compared to remdesivir blocks virus replication as a chain-terminator during RNA-dependent RNA polymerase mediated replication of the viral genome. Hence, also favipiravir acts broadly against several RNA viruses. Favipiravir was approved 2014 in Japan for the treatment of novel or re-emerging influenza viruses and since has also been approved in China and Russia. Favipiravir is thought to block SARS-CoV-2 replication and has shown some beneficial effects in clinical trials and was since approved to be used in a compassionate use program in Japan to treat COVID-19.

Preferably, a combination therapy of natural IgM/IgA antibodies with direct-acting antiviral drugs remdesivir or favipiravir exerts to synergistic effects due to reduction of virus replication-induced accumulation of oxidized phospholipids/OSE combined with enhanced clearance of accumulated oxidation products by oxidized phospholipid-/OSE-specific natural IgM/IgA antibodies.

Camostat mesylate was developed in Japan as a protease inhibitor in the 1980s and is used for the treatment of acute symptoms of chronic pancreatitis and postoperative reflux esophagitis. Camostat mesylate is active against the transmembrane protease serin 2 (TMPRSS2) which has been demonstrated to be required for efficient entry of SARS-CoV-1 and SARS-CoV-2 into lung cells.

Similar to camostat mesylate, the serin protease inhibitor nafamostat mesylate is approved for clinical use in Japan, also blocks TMPRSS2 and was shown to block SARS-CoV-2 infection in vitro. Both drugs block the activity of TMPRSS2 thereby preventing the cleavage of the viral S protein, a pre-requisite for virus entry into the host target cell. In the present invention, the combination of TMPRSS2 inhibiting drugs that function to inhibit virus replication such as camostat mesylate or nafamostat mesylate with oxidized phospholipids-/OSE-specific natural IgM/IgA antibodies preferably exerts synergistic therapeutic effects.

Since nafamostat mesylate also inhibits/antagonizes the accumulation of Receptor of Advanced Glycation Endproducts (RAGE) ligands, a combination with oxidized phospholipids-/OSE-specific natural IgM/IgA antibodies preferably has multiple synergistically acting effects by 1) reducing virus replication (through blocking TMPRSS2), 2) reducing RAGE ligands and 3) clearing oxidation products by oxidized phospholipids-/OSE-specific natural IgM/IgA antibodies.

Umifenovir (Arbidol) is a virustatics with dual direct-acting/host-targeting function that is approved in Russia and China for the treatment of respiratory virus infections including Influenza A and B. Umifenovir has shown potential to inhibit SARS-CoV-2 replication in vitro and has been suggested to have antiviral effects in vivo. Umifenovir blocks the fusion of the virus membrane with the target host cell membrane, hence blocks virus entry into the cell, but may also have effects in virus production and/or release due to its dual activity. It is a hydrophobic molecule that is capable to form aromatic stacking interactions with certain amino acid residues which likely contributes to its direct-acting antiviral activity, e.g., by binding to viral glycoproteins important for virus entry. Additionally, umifenovir has lipid binding capability and antiviral effects by binding directly the viral lipid-bilayer as well as by binding directly the plasma membrane of target cells and preventing virus uptake through endocytosis have been proposed. Intriguingly, umifenovir has been shown to have antioxidant potential and in comparison with the antioxidant Trolox showed prolonged antioxidant effects in vitro.

Preferably, umifenovir exerts synergistic effects in combination with oxidized phospholipid/OSE-specific natural IgM/IgA antibodies by two mechanism: 1) preventing virus replication and 2) reduction of oxidative stress through its antioxidant characteristics.

Stronger Neo-Minophagen C (SNMC) is a glycyrrhizin-containing preparation that is approved in Japan for the treatment of chronic hepatic diseases. Glycyrrhizin (GL) is a triterpene present in the roots and rhizomes of licorice (Glycyrrhiza glabra) and has been shown to have anti-inflammatory, anti-oxidative, and anti-viral effects. Licorice extract has been demonstrated to inhibit LDL oxidation and can exert antioxidative effects. In an ex vivo study it was shown that LDL isolated from normolipidemic subjects who were orally supplemented with licorice was more resistant to oxidation than LDL isolated before the licorice supplementation. Patients with hepatitis C virus infection under long-term treatment with SNMC developed less frequently liver cirrhosis as well as hepatocellular carcinoma. In addition, GL was demonstrated to efficiently block SARS-CoV-1 replication in vitro. GL is metabolized to the systemically active glycyrrhetinic acid (GA) which inhibits 11-beta-hydroxysteroid dehydrogenase (11 bHSD), and both GL and GA have demonstrated antiviral effects. Inhibition of 11 bHSD may lead to cortisol-mediated activation of mineralocorticoid receptors in aldosterone specific peripheral tissue, including the lung, kidney, as well as nasal and endothelial cells, resembling activity of high levels of aldosterone. Aldosterone infusion in animal models caused loss of the SARS-CoV-1 and -2 receptor ACE2 expression in the kidney, suggesting that GL/GA-induced inhibition of 11 bHSD may also reduce the expression of ACE2 in aldosterone specific tissue, hence reduce virus entry and spread. GA was also proposed to inhibit transcriptional expression of the protease TMPRSS2, a protein that is along with ACE2 required for efficient SARS-CoV-2 entry into cells. Importantly, while GL/GA may reduce supposedly protective ACE2 that reduces angiotensin II levels, at the same time both GL and GA have anti-inflammatory effects through toll-like receptor 4 (TLR4) antagonism and GL has been shown to inhibit ligand binding to RAGE.

Therefore, GL and GA exert antiviral effects by downmodulating the receptor (ACE2) and protease (TMPRSS2) required for efficient virus entry as well as anti-inflammatory effects that downmodulate the pro-inflammatory cytokine production and response.

Therefore, preferably, that reduction of virus replication and reduced production of oxidized phospholipids/OSE as well as the anti-inflammatory and antioxidative effects exerted by SNMC, combined with the clearance of oxidation products by oxidized phospholipids-/OSE-specific natural IgM/IgA antibodies exerts synergistic therapeutic effects.

As mentioned above, it has surprisingly been described in the present application that a subgroup of natural IgM and/or IgA antibodies can be used in the treatment or prevention of a disorder or a disease associated with/related to/caused by a natural IgM/IgA antibody deficiency (NAD) in a subject, i.e., human or humanized natural IgM and/or IgA antibody recognizing oxidized phospholipids and/or oxidation-specific epitopes. As explained above, the present invention is based on the surprising observation that the stimulatory effects of oxidized phospholipids (oxPL) and oxidation-specific epitopes (OSE) on macrophage activation and secretion of pro-inflammatory cytokines such as IL-6 contributes to the initiation of fatal cytokine release syndrome and the development of acute lung injury and ARDS in severe COVID-19 patients.

This observation leads to the first and second aspect of the present invention as defined above that a human or humanized natural IgM and/or IgA antibody recognizing oxidized phospholipids and/or oxidation-specific epitopes can be used in a method of treating or preventing a disorder or a disease associated with/related to/caused by a natural IgM/IgA antibody deficiency (NAD) in a subject.

Once these embodiments of the present invention found that the reduction of oxidized phospholipids and/or oxidation-specific epitopes by the use of human or a humanized natural IgM and/or IgA antibody recognizing oxidized phospholipids and/or oxidation-specific epitopes can be used to treat or prevent a disorder or a disease associated with/related to/caused by a natural IgM/IgA antibody deficiency (NAD) in a subject, without being bound by theory, this surprising observation opens for the possibility to modulate other immune cell functions normally important for an efficient anti-microbial immune response, ultimately countering the overall generation of oxidized phospholipids and/or oxidation-specific epitopes.

This is explained in the following.

For instance, it has been shown that the administration of oxPAPC rendered mice highly susceptible to E. coli peritonitis, as indicated by an accelerated mortality and enhanced bacterial outgrowth and dissemination. In this experimental setting, oxPAPC strongly inhibited the phagocytosing capacity of neutrophils and macrophages, although the macrophages were stimulated to release high amounts of IL-6 (Knapp et al., 2007, J Immunol. 178:993-1001). These data suggest that oxPLs formed during inflammatory reactions may contribute to mortality induced by Gram-negative sepsis via impairment of the phagocytic properties by macrophages, while simultaneously stimulating the secretion of pro-inflammatory cytokines such as IL-6. Furthermore, oxPL were shown to negatively regulate the function of dendritic cells (DCs). Maturation of DCs induced by pathogen-derived signals, for instance via TLR ligands, is a crucial step in the initiation of an adaptive immune response required to clear the infection. It has been demonstrated that oxPL, which are generated during infections, apoptosis, and tissue damage, interfere with DC activation and maturation by blocking TLR3- and TLR4-mediated induction of co-stimulatory molecules such as CD40, CD80, CD83, and CD86, and the secretion of the cytokines IL-12 and TNF. As a result, oxPAPC markedly reduced the costimulatory activity of DCs activated by TLR ligands, as indicated by reduced capacities to induce proliferation and effector cytokine production of antigen-specific T cells (Blüml et al., 2005, J Immunol. 175:501-508). In addition to inhibitory effects of oxPL on T cell function via blocking DC maturation, oxPL were also shown to directly inhibit the effector functions of T cells. Primary human T cells stimulated in the presence of different classes of oxPL, but not their native non-oxidized counterparts, showed impaired proliferation capacities, upregulation of activation markers such as MHC-II, CD25 and CD69, secretion of Th1 effector cytokines such as IFNγ, IL-2 and IL-10, and cytotoxic activities toward antigen-positive target cells (Seyerl et al., 2008, Eur J Immunol. 38:778-787).

Taken together, based on these observations, without being bound by theory, we understand that oxPL and OSE formed in the lungs of COVID-19 patients and in patients infected with other severe lung pathogens such as SARS-CoV and H5N1, inhibit important immune cell functions including the phagocytic capacity of macrophages and neutrophils, the maturation of DCs and their co-stimulatory activity, and the development of Th1-type responses and the effector phase of pathogen-specific cytotoxic T cells, and thereby additionally contribute to viral spreading, development of severe pneumonia and acute lung failure.

As outlined in detail above, to interfere with this inhibitory effects of oxPL and OSE on important immune cell functions, the administration of OSE-specific antibodies of the IgM and/or the IgA isotype, or plasma pools enriched for these, into affected patients restores, in terms of the present invention, the anti-viral immune response and clearance of the pathogen.

The finding of the present invention is all the more surprising because it is known to the skilled person in the art that OxPL and OSE exhibit anti-inflammatory and protective effects in the context of sepsis and acute injuries.

In fact, as already mentioned above, the anti-inflammatory effects of oxPL and OSE depend on their concentrations and include (1) inhibition of “sterile” acute lung injury induced by viral- and bacterial-derived inflammatory mediators (Ma et al., 2004, Am J Physiol Lung Cell Mol Physiol. 286:808-816; Nonas et al., 2006, Am J Respir Crit Care Med. 173:1130-1138); (2) inhibition of “aseptic” acute lung injury induced by injurious mechanical ventilation, and therefore it has been suggested that the use of 1-palmitoyl-2-(5,6-epoxyisoprostane E2)-sn-glycero-3-phosphorylcholine (PEIPC)- and 1-palmitoyl-2-(5,6-epoxycyclopentenone)-sn-glycero-3-phosphorylcholine (PECPC)-like stabilized compounds may show beneficial effects in other “aseptic” lung injury models such as ischemia/reperfusion (Nonas et al., 2008, Crit Care. 12:R27); and (3) inhibition of lung vascular leak and inflammation in the secondary acute lung injury induced by acute necrotizing pancreatitis (Li et al., 2007, Pancreas. 35:27-36).

These anti-inflammatory effects are mediated by enhanced endothelial barrier function (Birukov et al., 2004, Circ Res. 95:892-901; Birukova et al., 2007, Am J Physiol Lung Cell Mol Physiol. 292:924-935), induction of signaling pathways that lead to upregulation of anti-inflammatory genes, inhibition of pro-inflammatory gene expression (Eligini et al., 2002, Cardiovasc Res. 55:406-415; Ma et al., 2004, Am J Physiol Lung Cell Mol Physiol. 286:808-816; Otterbein et al., 2000, Nat Med. 6:422-428; Otterbein et al., 2003, Nat Med. 9:183-190), and prevention of the interaction of pro-inflammatory bacterial products with host cells (Bochkov et al., 2002, Nature. 419:77-81; Walton et al., 2003, Arterioscler Throm Vasc Biol. 23:1197-1203). However, in patients experiencing ARDS induced by infections with lung pathogens such as SARS-CoV-2, SARS-CoV and possibly H5N1 influenza viruses, we propose that multiple mechanisms implicated in the formation of ROS and oxidative stress convene in lungs of affected patients where oxPL and OSE accumulate to concentrations high enough to promote the biological effects described in the present invention.

In addition to the lack of sufficient amounts of oxPL- and OSE-specific natural antibodies of the IgM, and possibly the IgA isotype, that causes inefficient anti-inflammatory clearance of oxPL and OSE generated in inflamed tissues as described above in the present invention, without being bound by theory, this surprising observation opens for the possibility to modulate other immune cell functions normally important for an efficient anti-microbial immune response, ultimately countering the generation of oxidized phospholipids and/or oxidation-specific epitopes.

More specifically, once aware of the surprising finding of the present invention, it becomes readily evident that several other mechanisms can contribute to the massive formation of ROS and the accumulation of oxPL and OSE to pro-inflammatory concentrations in disorders or diseases associated with/related to/caused by a natural IgM/IgA antibody deficiency (NAD) in a subject.

These mechanisms include, but are not limited to,
(a) virus-induced downregulation of Angiotensin-Converting-Enzyme 2 (ACE2) receptor surface expression;
(b) accumulation of ligands for the Receptor-for-Advanced-Glycation-Endproducts (RAGE, also called AGER) and, consequently, enhanced RAGE signaling; and
(c) depletion of protective CD169+CD206+ alveolar macrophages expressing the Macrophage-Receptor-with-Collagenous-Structure (MARCO) receptor.

Hence, it is immediately evident that these mechanisms provide possibilities to supplement the use of the human or humanized natural IgM and/or IgA antibody recognizing oxidized phospholipids and/or oxidation-specific epitopes in a method of treating or preventing a disorder or a disease associated with/related to/caused by a natural IgM/IgA antibody deficiency (NAD) in a subject in terms of the present invention, thereby further countering the generation of oxidized phospholipids and/or oxidation-specific epitopes.

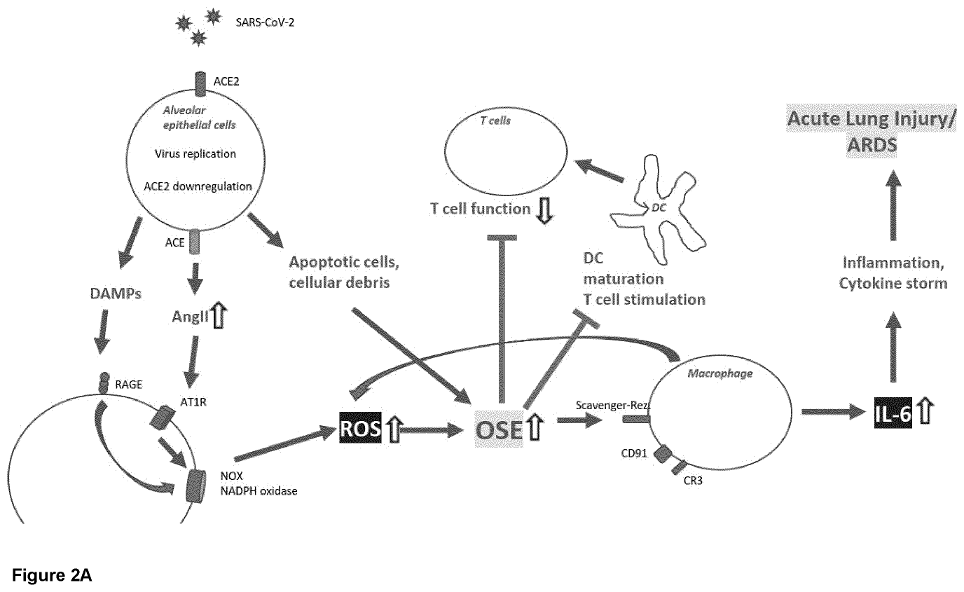

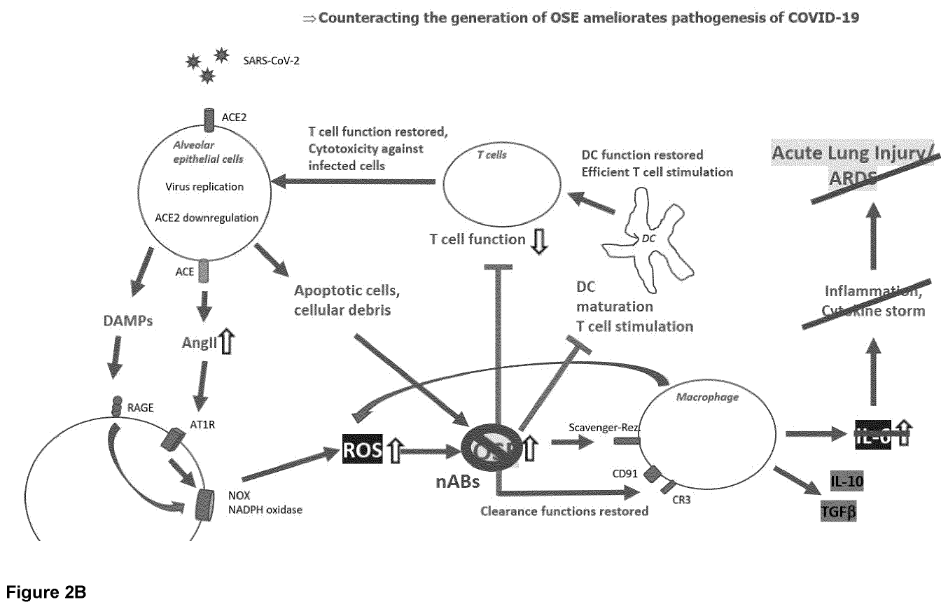

These additional mechanisms and possible ways to (further) counter the generation of oxidized phospholipids and/or oxidation-specific epitopes are illustrated in FIG. 2A and FIG. 2B, respectively.

Hence, in a preferred embodiment of the first and second aspect as defined above, said human or humanized natural IgM and/or IgA antibody recognizing oxidized phospholipids and/or oxidation-specific epitopes for use in a method of treating or preventing a disorder or a disease associated with/related to/caused by a natural IgM/IgA antibody deficiency (NAD) in a subject is to be administered in combination with:
(a) an inhibitor/antagonist of the Angiotensin-Converting-Enzyme (ACE), an inhibitor/antagonist of the Angiotensin-II-type 1 receptor (AT1R) and/or a compound that modulates the expression of the ACE2 receptor; and/or Ang(1-7), AT2R agonists and/or MAS-receptor agonists;
(b) a compound inhibiting/antagonizing/neutralizing ligands of Receptor of Advanced Glycation Endproducts (RAGE) and/or an inhibitor/antagonist of RAGE; and/or
(c) Granulocyte-Macrophage Colony-Stimulating Factor (GM-CSF) and/or a compound that increases the phagocytic activity of alveolar macrophages (AM), preferably azithromycin.

Preferably, such a combination therapy exerts synergistic effects on the treatment in accordance with the present invention.

As regards the preferred embodiments of such a combination therapy, the same applies, mutatis mutandis, as has been set forth above in the context of the first and/or second aspect of the present invention and/or the antibody or the pharmaceutical composition for use as defined above.

The combination therapy with:
(a) an inhibitor/antagonist of the Angiotensin-Converting-Enzyme (ACE), an inhibitor/antagonist of Angiotensin-II-type 1 receptor (AT1R) and/or a compound that modulates the expression of the ACE2 receptor; and/or Ang(1-7), AT2R agonists and/or MAS-receptor agonists;
(b) a compound inhibiting/antagonizing/neutralizing ligands of Receptor of Advanced Glycation Endproducts (RAGE) and/or an inhibitor/antagonist of RAGE; and/or
(c) Granulocyte-Macrophage Colony-Stimulating Factor (GM-CSF) and/or a compound that increases the phagocytic activity of alveolar macrophages (AM), preferably azithromycin;
   is explained in the following in more detail.

(a) An Inhibitor/Antagonist of the Angiotensin-Converting-Enzyme (ACE), an Inhibitor/Antagonist of Angiotensin-II-Type 1 Receptor (AT1R) and/or a Compound that Modulates the Expression of the ACE2 Receptor; and/or Ang(1-7), AT2R Agonists and/or MAS-Receptor Agonists

The ACE2 receptor was identified as the functional receptor for SARS-CoV-2 and SARS-CoV to enter host target cells. ACE2 is highly expressed in lung tissue, particularly in type-II alveolar (AT2) epithelial cells, modestly in bronchial and tracheal epithelial cells, and low in epithelial cells of heart, kidney and small intestine. In addition, ACE2 expression is normally upregulated in response to viral infection by type I and II interferons in primary human upper airway epithelial cells.

However, in a disorder or a disease associated with/related to/caused by a natural IgM/IgA antibody deficiency (NAD), preferably in SARS-CoV-2, the infection is different in that it does not induce a pronounced interferon response. Therefore, without being bound by theory, it is understood that this accounts for overall lower ACE2 levels.

ACE2 is highly homologous to ACE and both receptors play different key roles in regulating the renin-angiotensin-system, which controls arterial blood pressure, electrolyte homeostasis, as well as cardiovascular regulation and remodelling. ACE cleaves angiotensin-I (Ang-I) to generate angiotensin-II (Ang-II), whereas ACE2 inactivates Ang-II by cleavage to generate Ang(1-7) and is a negative regulator of the system. Ang-II binds to two downstream receptors, angiotensin II-type-1 receptor (AT1R) and angiotensin II-type-2 receptor (AT2R), while Ang(1-7) binds to the MAS receptor (MAS1 proto-oncogene, GPCR). Signaling via AT1R leads to vasoconstriction, hypertrophy, fibrosis and inflammation, whereas both AT2R and MAS receptors promote vasodilation, anti-fibrotic and anti-inflammatory functions.

Using genetically modified mice, it has been shown that ACE2 and AT2R protect mice from severe acute lung injury induced by acid aspiration or sepsis, while other components of the renin-angiotensin-system, including ACE, Ang-II and AT1R, promote disease pathogenesis, induce pulmonary vascular permeability and lung edemas, and impair lung function (Imai et al., 2005, Nature. 436:112-116).

In light of this, a loss of cell-surface expression of ACE2 due to its utilization as entry receptor for SARS-CoV-2 and SARS-CoV contributes to the pathology of lung injury additionally to the deleterious effects mediated by viral replication.

Thus, the administration of an inhibitor/antagonist of the Angiotensin-Converting-Enzyme (ACE), an inhibitor/antagonist of the Angiotensin-II-type 1 receptor (AT1R) and/or a compound that modulates (i.e., increases or decreases) the expression of the ACE2 receptor; and/or Ang(1-7), AT2R agonists and/or MAS-receptor agonists in a combination therapy with the above first and/or second aspect of the present invention and/or the antibody or the pharmaceutical composition for use as defined above, is a further means to counter the generation of oxidized phospholipids and/or oxidation-specific epitopes.

In the context of the present invention, a “modulated (i.e., increased or decreased) expression” preferably means that the expression and/or the activity of the ACE2 receptor in a given cell and/or organism is at least 10%, preferably at least 20%, more preferably at least 30% or 50%, even more preferably at least 70% or 80% and particularly preferred at least 90% or 100% higher (or lower) in the presence of the respective compound than in the corresponding cell and/or organism in the absence of the respective compound. In even more preferred embodiments the increase (or decrease) in expression may be at least 150%, at least 200% or at least 500%. In particularly preferred embodiments the expression is at least 10-fold, more preferably at least 100-fold and even more preferred at least 1000-fold higher (or lower) than in the corresponding cell and/or organism in the absence of the respective compound.

The term “decreased” expression of the ACE2 receptor also covers the situation in which the corresponding cell and/or organism does not express a corresponding ACE2 receptor so that the corresponding expression is zero. The term “increased” expression of the ACE2 receptor covers the situation that the ACE2 receptor is overexpressed and that the concentration of the overexpressed ACE2 receptor preferably is at least 5%, 10%, 20%, 30%, or 40% of the total cell's and/or organism's protein.

Methods for measuring the level of expression of a given ACE2 receptor in a cell and/or an organism are well known to the person skilled in the art. In one embodiment, the measurement of the level of expression is done by measuring the amount of the corresponding protein. Corresponding methods are well known to the person skilled in the art and include Western Blot, ELISA etc. In another embodiment the measurement of the level of expression is done by measuring the amount of the corresponding RNA. Corresponding methods are well known to the person skilled in the art and include, e.g., Northern Blot.

In fact, it has been shown that the intraperitoneal administration of purified SARS-CoV Spike (S)-protein, which is the crucial viral protein responsible for ACE2 binding, led to S-protein accumulation in the lungs of acid aspiration-treated mice. This induced downregulation of ACE2 expression and thereby augmented pathological changes in the lung parenchyma as assessed by increased lung elastance, vascular permeability and edema formation. Notably, Ang-II levels were significantly increased in the lung tissue of these mice due to S-protein-induced ACE2 downregulation and blocking AT1R signaling by the specific inhibitor Losartan markedly reduced the severity of acute lung injury and pulmonary edema (Kuba et al., 2005, Nat Med. 11:875-879). Similarly, ACE2 expression was downregulated in lung homogenates from mice infected with SARS-CoV as compared to uninfected controls. In support of these results, a small case study reported that plasma levels of Ang-II were markedly elevated and linearly associated with viral load and severity in lung injury in COVID-19 patients (Liu et al., Sci China Life Sci. 2020; 63:364-74). Interestingly, elevated serum Ang-II levels were also detected in patients infected with H5N1, an Influenza-A virus causing up to 70% lethality in humans due to induction of ARDS and respiratory failure. Further analyses in mice showed that infection with H5N1, but not H1N1 virus, caused downregulation of ACE2 expression in lung tissues and Ang-II serum levels were significantly increased in animals experiencing severe ARDS (Zhou et al., 2014, Nat Comm. 5:3594). This finding was surprising since H5N1 influenza viruses were not described to utilize ACE2 as entry receptor to infect host cells and this may explain why only H5N1, but not H1N1 influenza viruses, induced generation of ROS and accumulation of oxPL in lung tissue of infected mice that worsened the pathology of lung injury (Imai et al., 2008, Cell. 133:235-249).

The central role of ACE2 in the regulation of lung injury was further highlighted using mice deficient for ACE2, showing that lack of ACE2 augmented the severity of acute lung injury induced by acid aspiration, sepsis or H5N1, while administration of soluble recombinant human ACE2 had protective effects. Conversely, genetic deletion of ACE protected mice from severe lung injury.

Given that ACE2 is a key negative regulatory factor for severity of lung edema and acute lung failure, downregulation of ACE2 and increased levels of Ang-II a common molecular mechanism involved in the pathologies of different pathogen-induced lung diseases is proposed in the present invention. Interestingly, Ang-II is an efficient stimulator of the expression and activation of nicotinamide adenine dinucleotide phosphate-oxidase (NAD(P)H) in various cell types and one of the main effects of AT1R activation is the generation of ROS.

Therefore, it is proposed in the present invention that the elevated Ang-II concentrations observed in patients infected with SARS-CoV-2, SARS-CoV or H5N1, continuously drive the formation of ROS via AT1R activation, which in turn activates the peroxidation reaction of phospholipids present in cell-membranes and surfactant to generate oxPL and OSE that accumulate in lungs and possibly other inflamed tissues of infected patients. Under circumstances when the anti-inflammatory clearance of oxPL and OSE is defective, for instance because of reduced serum levels of oxPL- and OSE-specific natural antibodies of the IgM and possibly IgA isotype, as described in the present invention, oxPL and OSE accumulate to concentrations sufficient to promote the biological effects described in the present invention.

Therefore, the administration of oxPL- and OSE-specific IgM and possibly IgA antibodies, or plasma pools enriched for these, in combination with drugs that specifically inhibit and/or reduce expression of the receptors ACE and/or AT1R, and/or increase the expression of ACE2 or enhance ACE2-induced metabolism of Ang-II, is proposed to have additive or even synergistic effects to interfere with the generation of oxPL and OSE, and the pro-inflammatory and immunomodulatory functions of oxPL and OSE in COVID-19 patients and patients infected with other severe lung pathogens such as SARS-CoV and H5N1.

Thus, the administration of an inhibitor/antagonist of the Angiotensin-Converting-Enzyme (ACE), an inhibitor/antagonist of the Angiotensin-II-type 1 receptor (AT1R) and/or a compound that modulates the expression of the ACE2 receptor; and/or Ang(1-7), AT2R agonists and/or MAS-receptor agonists in a combination therapy with the above first and/or second aspect of the present invention and/or the antibody or the pharmaceutical composition for use as defined above, is a further means to counter the generation of oxidized phospholipids and/or oxidation-specific epitopes.

The skilled person is in a position to select an appropriate inhibitor/antagonist of Angiotensin-Converting-Enzyme (ACE), an inhibitor/antagonist of the Angiotensin-II-type 1 receptor (AT1R) and/or a compound that modulates (i.e., increases or decreases) the expression of the ACE2 receptor that has the desired capability in accordance with the present invention and in accordance with the above.

As examples, inhibitors/antagonists of the Angiotensin-Converting-Enzyme (ACE) and/or inhibitors/antagonists of the Angiotensin-II-type 1 receptor (AT1R) may be selected from the group consisting of Ramipril, Lisinopril, Olmesartan, Telmisartan, Losartan and Azilsartan.

As examples, compounds that modulates (i.e., increase or decrease) the expression of the ACE2 receptor may be selected from the group consisting of Thiazolidinediones and Ibuprofen.

Compounds that modulate (i.e., increase or decrease) the expression of the ACE2 receptor are well-known to the person skilled in the art and a compound that modulates (i.e., increases or decreases) the expression of the ACE2 receptor in accordance with the present invention is not limited to specific compounds. Rather, the skilled person is in a position to select a suitable compound that modulates (i.e., increases or decreases) the expression of the ACE2 receptor as long as it has the above functional capabilities.

Indeed, drugs to inhibit/antagonize the receptors ACE (e.g., Ramipril, Lisinopril) or AT1R (e.g., Olmesartan, Telmisartan, Losartan, Azilsartan) are known in the art and are clinically widely used for controlling acute and chronic hypertension, treating left ventricular dysfunction and heart failure, preventing strokes, and preventing and treating nephropathy in patients with hypertension or diabetes. Likewise, ACE2 expression levels can be increased by thiazolidinediones or ibuprofen and upregulation of ACE2 expression decreases Ang-II levels, which contributes to reduced accumulation of oxPL and OSE. Therefore, it is proposed in the present invention that drugs that up-regulate ACE2 expression are suitable for combination therapy with natural IgM and/or IgA antibodies targeting oxPL or OSE of late stage COVID-19, when the viral infection has been cleared.

Since patients with hypertension and cardiovascular diseases are likely treated with ACE and/or AT1R inhibitors, and this patient population show a high case fatality rate when infected with SARS-CoV-2, the concern has been raised whether the administration of such drugs may worsen the morbidity and mortality of COVID-19. This concern was based on the observation that multiple AT1R inhibitors (e.g. Olmesartan, Telmisartan, Losartan, Azilsartan) increased expression of ACE2 mRNA and protein levels in animal models of cardiovascular diseases, which in turn would facilitate SARS-CoV-2 entry into host cells and viral replication.

To analyze the effects of ACE and AT1R inhibitors on the clinical course of COVID-19, a retrospective, single-center analysis enrolling 112 patients revealed that the use of such drugs had no effects on the mortality rate of COVID-19 patients with cardiovascular diseases. In fact, mice pretreated with Olmesartan showed reduced formation of acute pulmonary edema and lung injury induced by acid aspiration and SARS-CoV S-protein, which is in line with a proposed protective effect of Losartan on the severity of lung injury.

To elucidate whether treatment of COVID-19 patients with modulators of the renin-angiotensin have beneficial or detrimental effects, multicenter, double-blinded placebo-controlled, randomized trials are currently conducted to investigate the effects of Losartan on mortality and hospital admission in COVID-19 patients requiring hospital admission (NCT04312009) and not requiring hospital admission (NCT04311177). Since recombinant human ACE2 showed beneficial effects in animal models for acute lung injury induced by acid aspiration, SARS-CoV S-protein or H5N1 infection, its use for the treatment of COVID-19 patients was tested in a randomized, controlled, pilot clinical study (NCT04287686). However, this study was withdrawn on Mar. 17, 2020 due to yet unclear reasons.

Based on the molecular mechanisms involved in the pathology of SARS-CoV-2, SARS-CoV and H5N1 influenza virus-induced lung injury, as described in the present invention, it is proposed in the present invention that oxPL- and OSE-specific IgM and possibly IgA antibodies, or plasma pools enriched for these, can be combined with human recombinant ACE2.

Preferably, such a combination therapy exerts synergistic effects on the treatment in accordance with the present invention, i.e., by interfering with the generation of oxPL and OSE in inflamed tissues, and the pro-inflammatory and immunomodulatory functions of oxPL and OSE in COVID-19 patients.

Thus, in a preferred embodiment, the administration of a ACE2, preferably human ACE2 and more preferably human recombinant ACE2, in a combination therapy with the above first and/or second aspect of the present invention and/or the antibody or the pharmaceutical composition for use as defined above, is a further means to counter the generation of oxidized phospholipids and/or oxidation-specific epitopes.

Indeed, the therapeutic use of recombinant ACE2 has been described in, e.g., Monteil et al., 2020, Cell. 181, 1-9; Imai et al., 2005, Nature. 436, 112-116; Zhou et al, 2014, Nat Comm. 5:3594 and Khan et al., 2017, Critical Care. 21:234.

Human ACE2 is known to the skilled person and has the amino acid sequence as shown in SEQ ID NO:18.

However, the present invention is not limited to the administration of the specific human ACE2 having the amino acid sequence as shown in SEQ ID NO:18 in terms of the present invention but also to ACE2 comprising an amino acid sequence with at least 70% identity to SEQ ID NO:18 wherein said ACE2 has the activity to cleave angiotensin-II into Ang(1-7).

In a more preferred embodiment, the ACE2 comprises an amino acid sequence which is at least n % identical to the above sequence of SEQ ID NO:18 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 having the activity to cleave angiotensin-II into Ang(1-7).

As regards the determination of the sequence identity, the same applies as has been set forth above.

Assays for determining the activity of ACE2 (and variants thereof) to activity to cleave angiotensin-II into Ang(1-7) are known in the art.

As described above, ACE2 converts Ang-II to Ang(1-7) and this activates the AT2R and MAS receptor, which decreases inflammation, thrombosis, pulmonary damage and fibrosis. Ang(1-7) also was shown to prevent activation of NAD(P)H oxidase (NOX) and has anti-oxidant effects. Activation of the MAS receptor has also been proposed as a therapy against sarcopenia by the company Biophytis, that has developed BIO101, a small-molecule agonist of the MAS receptor. A phase 2/3 clinical trial using BIO101 in COVID-19 patients is currently conducted.

Thus, in a preferred embodiment, the administration of Ang(1-7), AT2R agonists and/or MAS-receptor agonists in a combination therapy with the above first and/or second aspect of the present invention and/or the antibody or the pharmaceutical composition for use as defined above, is a further means to counter the generation of oxidized phospholipids and/or oxidation-specific epitopes, thereby reducing the accumulation of oxPL and OSE and the pro-inflammatory and immunomodulatory functions of oxPL and OSE in a subject having a disorder or a disease associated with/related to/caused by a natural IgM/IgA antibody deficiency (NAD), preferably in a COVID-19 patient.

Preferably, such a combination therapy exerts synergistic effects on the treatment in accordance with the present invention, i.e., by interfering with the generation of oxPL and OSE in inflamed tissues, and the pro-inflammatory and immunomodulatory functions of oxPL and OSE in a subject having a disorder or a disease associated with/related to/caused by a natural IgM/IgA antibody deficiency (NAD), preferably in a COVID-19 patient.

Angiotensin(1-7) (or Ang(1-7)) is known to the skilled person as an active heptapeptide of the renin-angiotensin system (RAS) having the peptide sequence Asp-Arg-Val-Tyr-Ile-His-Pro). Angiotensin (1-7) is a vasodilator agent that plays important roles in cardiovascular organs, such as heart, blood vessels, and kidneys having functions frequently opposed to those attributed to the major effector component of the RAS, angiotensin II (Ang II). Ang (1-7) has been shown to have anti-oxidant and anti-inflammatory effects. Ang (1-7) plays protective roles in cardiomyocytes of spontaneously hypertensive rat by increasing the expression of endothelial and neuronal nitric oxide synthase enzymes leading to augmented production of nitric oxide. Ultimately, Ang (1-7) evokes anti-arrhythmogenic effects in animal models. In blood vessels, Ang (1-7) induces the release of vasodilators such as prostanoids and nitric oxide.

The skilled person is in a position to select an appropriate agonist of AT2R or MAS-receptor that has the desired capability in accordance with the present invention and in accordance with the above.

As examples, agonists of Ang(1-7) or AT2R or MAS-receptor may be selected from the group consisting of:

AT2R-agonists: Ang(1-7), peptide agonists such as CG42112A and dKcAng(1-7) (LP2-3), β-amino acid substituted Angiotensin II, gamma-turn mimetics incorporated into Angiotensin II; small molecule agonists such as Compound 21; and agonistic monoclonal antibodies.

MAS-receptor agonists: Ang(1-7), Peptide agonists such as TXA127 (Ang(1-7)), cyclic Ang(1-7), Ang(1-6)-O-Ser-Glc-NH2 (PNA5), hydroxypropyl-β-cyclodextin (HPβCD)/Ang(1-7), CGEN-856 and CGEN-857; small molecule agonists such as Sarconeos (BIO101) and AVE0991; and agonistic monoclonal antibodies.

(b) a Compound Inhibiting/Antagonizing/Neutralizing Ligands of Receptor of Advanced Glycation Endproducts (RAGE) and/or Inhibitors/Antagonists of RAGE

The receptor for advanced glycation endproducts (RAGE) is a pro-inflammatory PRR and has been implicated in the pathogenesis of numerous inflammatory diseases. RAGE exists in the body in two main forms: membrane-bound RAGE (mRAGE) and soluble RAGE (sRAGE). mRAGE possesses signaling activity in response to ligand binding, whereas sRAGE functions as a decoy receptor that sequesters RAGE ligands and thereby counteracts mRAGE signaling and inflammatory responses. In adult tissue at baseline, RAGE is constitutively highly expressed in the lung, where it is primarily localized to the basal membrane of type-1 alveolar epithelial (AT1) cells. In addition to expression in lung epithelium, RAGE expression has also been noted in vascular smooth muscle cells, airway smooth muscle cells, endothelial cells, neurons, and immune cells such as macrophages, DCs, eosinophils, T cells and B cells. However, many of the cells and tissues induce RAGE expression only when they are activated to do so, such as by local availability of RAGE ligands. RAGE can bind a large variety of endogenous ligands that are classified as DAMPs, including advanced glycation endproducts (AGEs), S100/calgranulin proteins, high mobility group box 1 protein (HMGB1), DNA or RNA, OSEs and phosphatidylserine.

AGEs are the result of a non-enzymatic Maillard reaction between the carbonyl group on an aldose sugar (commonly glucose) and amino groups on proteins or phospholipids, and AGEs are found at increased levels in patients with diabetes due to high blood glucose levels. Notably, age and oxidative stress also elevate AGE levels. S100 proteins are small calcium-binding proteins that localize to sites of inflammation, where they are released by activated inflammatory cells, and numerous S100 proteins can activate RAGE in a variety of tissues to initiate an inflammatory response. HMGB1 is a nuclear protein normally involved in chromatin remodelling, however, it can also be passively released from damaged cells as a pro-inflammatory alarm in. In addition, neutrophils, macrophages, natural killer cells, and DCs can actively secrete HMGB1, which is often associated with DNA. Neutrophiles secrete NETs (neutrophile extracellular traps), which are composed of decondensed DNA bound by histones and HMGB1.

NETosis, the secretion of NETs by neutrophils, has been proposed to be increased in severe COVID-19 courses and serum concentrations of NETosis products (e.g., cell-free DNA) correlate with COVID-19 severity (Middleton et al., 2020, Blood. doi: 10.1182/blood.2020007008). Through electrostatic interactions between a positive cavity on RAGE and the negative charges on the backbone of nucleic acids, RAGE can also directly bind DNA or RNA to facilitate their uptake into the cell to promote inflammatory responses. Activation of RAGE causes sustained nuclear factor kappa B (NFκB) signaling, and a large by-product of RAGE signaling is the excessive formation of ROS, which also contributes to NFκB activation and promote other inflammatory mechanisms such as increased vascular cell adhesion molecule 1 (VCAM-1) expression, generation of oxPL and OSE, or cellular apoptosis.

The presence of RAGE ligands in the extracellular environment has been shown to upregulate RAGE expression, which comes from the fact that NFκB can directly bind to the gene encoding RAGE to promote RAGE expression and leads to further amplification of inflammatory signaling cascades. Importantly, RAGE ligands are not degraded or altered to prevent further signaling when they bind and signal through RAGE. Therefore, enhanced RAGE signaling generates more RAGE ligands such as AGEs and OSEs due to generation of ROS and oxidative stress, and as ligands accumulate, they continuously amplify the inflammatory response by pooling in the inflamed region, thereby driving chronic pathological inflammation in a variety of respiratory diseases.

The central role of RAGE in the pathology of lung injury is supported by the observation that RAGE-deficient mice were protected from hyperoxia-induced acute lung injury and mortality, suggesting that intact RAGE signaling promotes lung inflammation and respiratory failure. RAGE-deficient mice were also partially protected from lung injury following gram-negative (E. coli) or gram-positive (S. pneumoniae) bacterial challenges. In humans, systemic and alveolar levels of HMGB1, S100A12, and sRAGE from damaged AT1 cells are increased in patients with ARDS, and plasma sRAGE levels correlate with severity of lung injury and increased mortality. The destructive positive feedback loop between RAGE, NFκB, ROS and the generation of more RAGE ligands can be attenuated by the release of sRAGE either from damaged cells, through alternative splicing events, or the proteolytic cleavage of mRAGE by ADAM10 or matrix metalloproteinase 9. Interestingly, in mice it has been shown that mRAGE also binds the integrin Mac-1 (aMb2, CD11b/CD18) on leukocytes to facilitate their recruitment to inflamed tissue. Given that mouse B1 cells constitutively express Mac-1, and influenza virus infections led to recruitment of B1 cells in lung tissue, where they secreted protective natural IgM antibodies, it is conceivable that B1 cell recruitment into inflamed tissue may be driven by activated RAGE. Furthermore, many RAGE ligands such as AGEs, OSEs, DNA or RNA, and phosphatidylserine, are well-described targets of B1 cell-derived natural antibodies in mice and humans. Taken together, these observations suggest that RAGE plays a central role in pro-inflammatory immune responses in inflamed tissues that may be further exacerbated in situations when RAGE-ligands such as AGEs, DNA/RNA, OSE and apoptotic cells cannot be cleared efficiently, for instance because of reduced levels natural antibodies of the IgM and possibly the IgA isotype.

Thus, it is proposed in the present invention that accumulation of RAGE ligands drives the pathogenesis and ARDS in severe COVID-19 patients who show evidence of increased oxidative stress, accumulation of AGEs due to advanced age and comorbidities such as diabetes, and reduced levels of natural IgM and/or IgA antibodies that normally contribute to anti-inflammatory clearance of RAGE ligands.

Accordingly, in a preferred embodiment, the administration of a compound inhibiting/antagonizing/neutralizing ligands of Receptor of Advanced Glycation Endproducts (RAGE) in a combination therapy with the above first and/or second aspect of the present invention and/or the antibody or the pharmaceutical composition for use as defined above, is a further means to counter the generation of oxidized phospholipids and/or oxidation-specific epitopes, thereby reducing the accumulation of oxPL and OSE and the pro-inflammatory and immunomodulatory functions of oxPL and OSE in a subject having a disorder or a disease associated with/related to/caused by a natural IgM/IgA antibody deficiency (NAD), preferably in a COVID-19 patient.

Preferably, such a combination therapy exerts synergistic effects on the treatment in accordance with the present invention, i.e., by interfering with the generation of oxPL and OSE in inflamed tissues, and the pro-inflammatory and immunomodulatory functions of oxPL and OSE in a subject having a disorder or a disease associated with/related to/caused by a natural IgM/IgA antibody deficiency (NAD), preferably in a COVID-19 patient.

The skilled person is in a position to select an appropriate a compound inhibiting/antagonizing/neutralizing ligands of Receptor of Advanced Glycation Endproducts (RAGE) that has the desired capability in accordance with the present invention and in accordance with the above.

As examples, compounds inhibiting/antagonizing/neutralizing ligands of Advanced Glycation Endproducts (RAGE) may be selected from the group consisting of Nafamostat Mesilate (NM), Gabexate Mesilate (GM), Sivelestat, Atorvastatin, Simvastatin, Methotrexate (MTX), Alagebrium (ALT-711), SYI-2074 (ALT-2074), and Paquinimod (ABR-215757).

Further examples of compounds inhibiting/antagonizing/neutralizing ligands of Advanced Glycation Endproducts (RAGE) may be selected from the group consisting of small molecules such as Glycyrrhizin (GL), Carbenoxolone (CBX), Tanshinone I, Tanshinone IIa, Cryptotanshinone, sodium-sulfonate-derivative-of-Tanshinone Ila (TSNIIA-SS), Epigallocatechnin-3-gallate (EGCG), Quercetin, Lycopene, Nafamostat Mesilate (NM), Gabexate Mesilate (GM), Sivelestat, Atorvastatin, Simvastatin, Ethyl Pyruvate (EP) and derivates, Methotrexate (MTX), Alagebrium (ALT-711), SYI-2074 (ALT-2074), Cromolyn, Paquinimod (ABR-215757) and Tasquinimod (ABR-215050); recombinant proteins such as Box-A Protein (truncated N-terminal domain of HMGB1) and Box-A-Acidic Tail-Fusion Protein; Peptides such as S100P-derived peptides, Carnosine, Homocarnosine, Anserine, Glutathione; and monoclonal antibodies specific for carboxymethyllysine (CML)-modified proteins, lipids or other molecules, carboxyethyllysine (CEL)-modified proteins, lipids or other molecules, Glucose-modified proteins, lipids or other molecules, HMGB1, and S100 proteins.

In addition, the accumulation of RAGE ligands can contribute to the development of long-lasting autoimmune IgGs, explaining the long-lasting symptoms in some COVID-19 convalescent patients.

In fact, it has surprisingly been found in the present invention (see Example 22 and FIG. 3) that in patients with severe COVID-19, autoimmune antibodies are generated. These data support that the lack of natural antibodies (nABs) can result in the development of autoimmune antibodies during severe COVID-19 courses. The presence of these autoimmune antibodies provides evidence for recurring or long-lasting COVID-19 disease symptoms, supporting that sufficient levels of natural antibodies, provision of monoclonal natural IgMs or IgAs, or preparations enriched for natural antibodies (e.g. Pentaglobin®) in terms of the present invention can prevent the generation or reduce the levels of autoimmune antibodies.

In this scenario, it is proposed that the administration of oxPL- and OSE-specific IgM and possibly IgA antibodies, or plasma pools enriched for these, counteracts the destructive inflammatory immune response by promoting the save clearance of RAGE ligands and thereby represents an important mechanism involved in the resolution of the self-amplifying pro-inflammatory signaling cascade driven by RAGE. In addition, it is proposed in the present invention that the administration of oxPL- and OSE-specific IgM and possibly IgA antibodies, or plasma pools enriched for these, in combination with selective inhibitors or antagonists of RAGE, or in combination with antibodies specific for RAGE ligands such as anti-HMGB1 antibodies, has synergistic effects to interfere with the generation of oxPL, OSE and other RAGE ligands in inflamed tissues, and the pro-inflammatory and immunomodulatory functions of oxPL, OSE and other RAGE ligands, in COVID-19 patients.

Accordingly, in a preferred embodiment, the administration of an inhibitor/antagonist of RAGE in a combination therapy with the above first and/or second aspect of the present invention and/or the antibody or the pharmaceutical composition for use as defined above, is a further means to counter the generation of oxidized phospholipids and/or oxidation-specific epitopes, thereby reducing the accumulation of oxPL and OSE and the pro-inflammatory and immunomodulatory functions of oxPL and OSE in a subject having a disorder or a disease associated with/related to/caused by a natural IgM/IgA antibody deficiency (NAD), preferably in a COVID-19 patient.

Preferably, such a combination therapy exerts synergistic effects on the treatment in accordance with the present invention, i.e., by interfering with the generation of oxPL and OSE in inflamed tissues, and the pro-inflammatory and immunomodulatory functions of oxPL and OSE in a subject having a disorder or a disease associated with/related to/caused by a natural IgM/IgA antibody deficiency (NAD), preferably in a COVID-19 patient.

The skilled person is in a position to select an appropriate inhibitor/antagonist of RAGE that has the desired capability in accordance with the present invention and in accordance with the above.

As examples, inhibitors/antagonists of RAGE may be selected from the group consisting of small molecules such as TTP488 (Azeliragon) and derivates, FPS-ZM1; antagonistic RAGE-specific peptides; and antagonistic RAGE-specific monoclonal antibodies.

(c) Granulocyte-Macrophage Colony-Stimulating Factor (GM-CSF) and/or a Compound that Increases the Phagocytic Activity of Alveolar Macrophages (AM), Preferably Azithromycin

The lung harbours a large number of macrophages including the two main populations interstitial (IM) and alveolar macrophages (AM) that reside in different anatomical compartments.

AMs typically express the master transcription factor peroxisome proliferator-activated receptor γ (PPARγ), a key regulator of lipid metabolism, which is induced by the cytokine granulocyte-macrophage colony-stimulating factor (GM-CSF). TGFβ is another cytokine required for AM development and homeostasis and in contrast to GM-CSF, which is secreted by type-2 alveolar (AT2) epithelial cells, TGFβ produced by AM themselves supports their homeostasis.

Mouse AM originate from fetal monocytes that seed the lung and differentiate after birth into mature AM under the influence of GM-CSF, TGFβ and PPARγ. Accumulating evidence supports the concept that in the human steady-state lung, AM are maintained by their self-renewal capacity under the critical influence of GM-CSF and PPARγ which is conserved between mouse and humans. AM are located in the lumen of alveoli, where the gas exchange takes place at the alveolar-capillary membrane, and their strategic localization allows them to clear the airway of pathogens, apoptotic cells, and other airborne particles through phagocytosis, which is essential to maintain the vital oxygen uptake. AM express several factors that promote immune tolerance, such as TGFβ, as well as inhibitory receptors, restraining their pro-inflammatory activity under steady-state conditions.

An essential function of AM is their ability to catabolize surfactant and lack of GM-CSF or PPARγ, and hence the lack of AM, results in inflammatory lung diseases due defective surfactant metabolism. The ability of AM to catabolize surfactant relies on the expression of scavenger receptors such as SR-AI and macrophage-receptor-with-collagenous-structure (MARCO), which bind to oxPL present in surfactant and facilitate their uptake into intracellular compartments, where the lipids are used for energy metabolism by the fatty acid oxidation pathway. In contrast, IM and monocyte-derived macrophages do not express MARCO under steady-state conditions, or to a much lesser extent, and they do not use lipids for energy metabolism since they rely on glycolysis. The importance of MARCO for AM-mediated clearance of oxPL present in surfactant has been demonstrated using genetically modified mice lacking MARCO expression. In these mice, ozone exposure led to excessive formation of different classes of oxPL in surfactant, which promoted an inflammatory environment and acute lung injury, whereas MARCO-expressing wildtype mice were protected from severe lung injury induced by ozone instillation. The study further showed that intratracheal instillation of oxPL caused substantial neutrophil influx in MARCO-deficient mice, but had no effect in wildtype mice, consistent with improved uptake of oxPL by normal AM compared to MARCO-deficient AM (Dahl et al., 2007, J Clin Invest. 117:757-764).

Thus, these results indicate that AM internalize potentially pro-inflammatory oxPL via MARCO without engaging the typical detrimental response in the lungs mediated by pro-inflammatory monocyte-derived macrophages or neutrophils. These data also suggest that reduced levels of AM in lungs predispose individuals for the development of severe lung injury and ARDS induced by infections with lung pathogens such as SARS-CoV-2, SARS-CoV or H5N1 influenza virus.

In fact, single-cell RNA-sequencing analyses of immune cells contained in BAL fluid derived from patients with varying severity of COVID-19 and from healthy human donors revealed that classical MARCO-positive AM were depleted in lungs of critically ill patients, whereas AM were abundant in BAL fluid derived from mild COVID-19 cases and healthy donors. BAL fluid of patients with severe COVID-19 pathogenesis contained higher proportions of pro-inflammatory monocyte-derived macrophages and neutrophils, and lower proportions of DCs and effector T cells compared to those with mild disease. Furthermore, the lung macrophages in severe COVID-19 patients expressed markedly higher levels of inflammatory cytokines and chemokines such as IL-6, IL-1β IL-8, TNFα, CCL2, CCL3, CCL4 and CCL7 compared to macrophages contained in BAL fluid from mild COVID-19 cases.

Therefore, the lack of AM contributes to the pro-inflammatory environment in the lung of severe COVID-19 patients responsible for the development of lethal ARDS, although the mechanism of AM depletion remains unclear. Since AM are in close proximity to AT1 and AT2 pneumocytes, and express low levers of ACE2 receptors, it is possible that AM become directly infected by SARS-CoV-2 causing their depletion as observed in severe COVID-19 patients. In support of this notion, the human coronavirus E229 has been shown to infect AM, which led to the secretion of proinflammatory cytokines such as CCL4, CCL5 and TNFalpha. Similarly, diffuse alveolar damage was associated with SARS-CoV-1 infection of AT2 pneumocytes as well as AM in the lung of a 73 year old man, 7 days after hospitalization (Shieh et al., 2005, Hum Pathol. 36:303-309). AT2 pneumocytes are the main producers of surfactant and we propose that infection of this cell type triggers an increased accumulation of surfactant with higher levels of oxPL and OSE due to enhanced oxidative stress and defective clearance mechanisms.

Since SARS-CoV-2 uses the same entry receptor (ACE2) and the cellular proteases (Furin, TMPRSS2) for S-mediated virus entry, it is plausible that also SARS-CoV-2 infects both AT2 pneumocytes and AM, which is proposed to contribute to increased accumulation of surfactant with inflammatory levels of oxPL and OSE, cell debris and the depletion of AM as a result of their infection.

In support of a direct infection by SARS-CoV-2, populations of CD169⁺ lymph node subcapsular and splenic marginal zone macrophages express the SARS-CoV-2 entry receptor ACE2 and it was shown that these macrophages contained SARS-CoV-2 nucleoprotein (NP) (Feng et al, 2020, medRxiv, doi: https://doi.org/10.1101/2020.03.27.20045427).

Alternatively, studies in mice have shown that AM numbers are reduced during multiple forms of tissue injury, e.g., after ionizing radiation, viral infection, and LPS-induced lung injury. It has been proposed that damage to the lung epithelium may lead to loss of integrin dependent TGFβ activation in AM, thereby causing a reduction in AM. In case of COVID-19, it is proposed that reduced numbers of AT2 cells due to SARS-CoV-2 replication dampens local GM-CSF production, which in turn leads to depletion of AM. Alternatively, mouse studies showed that AM are naturally depleted with advanced ageing, leading to a decrease in their number in older mice, and it has been suggested that the contribution of HPSC-derived monocytes to the AM compartment steadily increases with age. The proliferative capacity of AM and the clearance of apoptotic neutrophils by AM is impaired in old mice. Furthermore, AM from old mice are refractory to IFNg, resulting in impaired killing of phagocytosed pathogens.

Therefore, it is plausible that age-related changes in lung macrophages favour a state of impaired host defence coupled with monocytic and neutrophilic inflammation causing excessive tissue damage in the lung. Interestingly, in mice it has been shown that B1 cells residing in the pleural space mobilize to the lung in response to an infection, where they give rise to a population called innate-response-activator (IRA) B cells that produce high amounts of IL-3 and GM-CSF, the latter of which induces enhanced secretion of natural IgM antibodies in an autocrine mechanism. The importance of IRA B cells for clearing infections has been demonstrated using mice lacking IRA B cells due to a B cell-restricted GM-CSF deficiency, as these mice were particularly prone to bacterial sepsis as indicated by a significantly higher mortality rate than in control mice, which was associated with pronounced inflammation, induction of a cytokine release syndrome, and more severe bacteremia, which led to septic shock, multiorgan failure and death.

Also in humans, GM-CSF-producing IRA B cells possessing the phenotype CD5+CD19+CD20+IgM+IgD+ were found to reside in tonsils, which function as a first line of defence from infections of the upper respiratory tract. Given that B1 cells are reduced in aged mice and humans, the absence of GM-CSF producing IRA B cells in lungs of infected individuals not only results in reduced levels of protective natural antibodies of the IgM and/or IgA isotype, but may also contributes to AM depletion and the pathogenesis of lung injury. In support of this model, a recent study described that low plasma levels of IL-3 were associated with increased severity and mortality during SARS-CoV-2 infections, indicating that IRA B cells are absent in critically ill COVID-19 patients.

Therefore, it is proposed in the present invention that the administration GM-CSF to COVID-19 patients may have beneficial effects in that it restores the AM population and the production of protective natural IgM and possibly IgA antibodies by B1 and/or IRA B cells.

Accordingly, in a preferred embodiment, the administration of a GM-CSF in a combination therapy with the above first and/or second aspect of the present invention and/or the antibody or the pharmaceutical composition for use as defined above, is a further means to counter the generation of oxidized phospholipids and/or oxidation-specific epitopes, thereby reducing the accumulation of oxPL and OSE and the pro-inflammatory and immunomodulatory functions of oxPL and OSE in a subject having a disorder or a disease associated with/related to/caused by a natural IgM/IgA antibody deficiency (NAD), preferably in a COVID-19 patient.

Preferably, such a combination therapy exerts synergistic effects on the treatment in accordance with the present invention, i.e., by interfering with the generation of oxPL and OSE in inflamed tissues, and the pro-inflammatory and immunomodulatory functions of oxPL and OSE in a subject having a disorder or a disease associated with/related to/caused by a natural IgM/IgA antibody deficiency (NAD), preferably in a COVID-19 patient.

GM-CSF is known to the skilled person and has the amino acid sequence as shown in SEQ ID NO:19.

However, the present invention is not limited to the administration of the specific GM-CSF having the amino acid sequence as shown in SEQ ID NO:19 in terms of the present invention but also to GM-CSF comprising an amino acid sequence with at least 70% identity to SEQ ID NO:19 wherein said GM-CSF has the activity to induce proliferation of TF-1 cells.

In a more preferred embodiment, the GM-CSF comprises an amino acid sequence which is at least n % identical to the above sequence of SEQ ID NO:19 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 wherein said GM-CSF has the activity to induce proliferation of TF-1 cells.

As regards the determination of the sequence identity, the same applies as has been set forth above.

Assays for determining the activity of GM-CSF (and variants thereof) to induce proliferation of TF-1 cells are known in the art.

Likewise, treatment of 1438 hospitalized COVID-19 patients with azithromycin showed beneficial effects. The hazard ratio for azithromycin treated patients was 0.56 [95% CI, 0.26-1.21]. The probability of death was slightly reduced for azithromycin treated patients (10.0% [95% CI, 5.9%-14.0%]) as compared to untreated (12.7% [95% CI, 8.3%-17.1%]) (Rosenberg et al., 2020, JAMA. 323:2493-2502). Clearly further studies are required, but these results are intriguing, since azithromycin was shown to enhance phagocytosis of apoptotic bronchial epithelial cells by alveolar macrophages (Hodge et al., 2006, Eur Respir J. 28:486-495 Hodge et al., European Respiratory Journal, 2006).

Accordingly, in a preferred embodiment, the administration of a compound that increases the phagocytic activity of alveolar macrophages (AM), preferably azithromycin a combination therapy with the above first and/or second aspect of the present invention and/or the antibody or the pharmaceutical composition for use as defined above, is a further means to counter the generation of oxidized phospholipids and/or oxidation-specific epitopes, thereby reducing the accumulation of oxPL and OSE and the pro-inflammatory and immunomodulatory functions of oxPL and OSE in a subject having a disorder or a disease associated with/related to/caused by a natural IgM/IgA antibody deficiency (NAD), preferably in a COVID-19 patient.

Preferably, such a combination therapy exerts synergistic effects on the treatment in accordance with the present invention, i.e., by interfering with the generation of oxPL and OSE in inflamed tissues, and the pro-inflammatory and immunomodulatory functions of oxPL and OSE in a subject having a disorder or a disease associated with/related to/caused by a natural IgM/IgA antibody deficiency (NAD), preferably in a COVID-19 patient.

The skilled person is in a position to select an appropriate compound that increases the phagocytic activity of alveolar macrophages (AM) that has the desired capability in accordance with the present invention and in accordance with the above.

In a third aspect, the present invention relates to a vaccine comprising a compound that induces the generation of natural IgM and/or IgA antibodies for use in a method of reducing or preventing clinical signs or disease associated with/related to/caused by natural IgM/IgA antibody deficiency (NAD) in a subject, wherein said vaccine comprises a pharmaceutically acceptable carrier or excipient.

A vaccine commonly refers to an “immunogenic composition” that comprises at least one agent that resembles a disease-causing virus or microorganism. Thus, a vaccine is generally a biological preparation that normally provides active acquired immunity to a particular infectious disease. A vaccine is often made from weakened or killed forms of the microbe, its toxins, or one of its surface proteins. The agent stimulates the body's immune system to recognize the agent as a threat, destroy it, and to further recognize and destroy any of the microorganisms or viruses associated with that agent that it may encounter in the future.

A vaccine or an immunogenic portion thereof commonly elicits an immunological response (cellular or antibody-mediated immune response) in the host to the composition.

The term “vaccine” as used in specific aspects of the present invention refers, however, to a pharmaceutical composition which does not (predominantly) elicit an immunological response in an animal or a subject but comprises a compound that induces the generation of natural IgM and/or IgA antibodies recognizing oxidized phospholipids and/or oxidation-specific epitopes.

Epidemiological data indicate that in some cases populations being vaccinated against a defined pathogen may have acquired cross-immunity towards also other non-related pathogens. A prominent recent example is COVID-19. Here, observation data indicate that in disease populations with previous vaccination against e.g. pneumococcus pneumoniae or Bacillus tuberculosis COVID-19 disease may be both less frequent and less severe. Although potential cross-immunity on the basis of prior vaccination in these populations has been discussed there is thus far no convincing explanation for the actual cause of this protection. We suggest the reason for better protection towards COVID-19 in e.g. BCG or pneumococcus vaccinated individuals to be related to vaccine induced OSE-specific antibodies which provide cross-immunity protection towards non-related pathogens. Consequently, vaccines being capable of inducing not only specific immune responses towards the vaccinated pathogen but also induce OSE-specific antibodies may define a new class of reagents for active immunotherapy interventions.

Without being bound to theory, vaccination strategies aimed at induction of endogenous production of OSE-specific IgM or IgA antibodies are be applied to protect healthy individuals from chronic sterile inflammation diseases or pathogen-induced severe forms of ALI and ARDS.

In mice, vaccination with Mycobacterium tuberculosis lipids or the Bacillus Calmette-Guerin (BCG) vaccine stimulated B1 cells to produce natural IgM antibodies possessing specificities for the phosphocholine head group of phosphatidylcholine and cardiolipin (Russo and Mariano, 2010, Immunobiology, Vol. 215 (12)) (Ordonez, Savage et al., 2018, Immunology, Vol.)). Similarly, immunization of atherosclerosis-prone mice with inactivated pneumococcal extracts resulted in high levels of phosphocholine-specific IgM antibodies that had atheroprotective functions because they blocked the uptake of oxLDL by macrophages (Binder, Horkko et al., 2003, Nat Med, Vol. 9 (6)). These studies show molecular mimicry between OSE and epitopes within cell wall components of bacteria such as M. tuberculosis and S. pneumoniae and indicate that vaccination of human individuals with BCG or Pneumovax23 may induce immune responses that protect from chronic inflammation diseases such as atherosclerosis, SLE, MS, and AD, but also from severe courses of pathogen-induced ALI and ARDS. Interestingly, a previous exposure to the BCG vaccine seems to protect humans from fatal COVID-19, since the mortality rate in recently vaccinated individuals is reduced compared to non-vaccinated humans.

A vaccine may additionally comprise further components typical to pharmaceutical compositions as defined above. By way of distinction the immunologically active component of a vaccine may comprise complete virus particles or complete bacteria in either their original form or as attenuated particles or attenuated bacteria.

In another form the immunologically active component of a vaccine may comprise appropriate elements of the organisms (subunit vaccines) whereby these elements are generated either by destroying the whole particle or bacteria or the growth cultures containing such particles and optionally subsequent purification steps yielding the desired structure(s), or by synthetic processes including an appropriate manipulation by use of a suitable system based on, for example, bacteria, insects, mammalian, or other species plus optionally subsequent isolation and purification procedures, or by induction of the synthetic processes in the animal needing a vaccine by direct incorporation of genetic material using suitable pharmaceutical compositions (polynucleotide vaccination). A vaccine may comprise one or simultaneously more than one of the elements described above. The term “vaccine” as used in specific aspects of the present invention describes a modified live, attenuated vaccine. In further specific aspects of the present invention the vaccine may inter alia be a live vaccine, a live-attenuated vaccine, an inactivated vaccine, or a conjugate vaccine.

Various physical and chemical methods of inactivation are known in the art. The term “inactivated” refers to a previously virulent or non-virulent virus or bacteria that has been irradiated (ultraviolet (UV), X-ray, electron beam or gamma radiation), heated (for instance for 30 min to several hours at a temperature between 55° C. and 65° C., e.g. 3 h at 56° C.), or chemically treated to inactivate, kill, such virus or bacteria while retaining its immunogenicity.

More particularly, the term “inactivated” in the context of a virus means that the virus is incapable of replication in vivo or in vitro. For example, the term “inactivated” may refer to a virus that has been propagated in vitro, and has then been deactivated using chemical or physical means so that it is no longer capable of replicating. In another example, the term “inactivated” may refer to a virus and/or a bacteria that has been propagated, and then deactivated using chemical or physical means resulting in a suspension of the virus, fragments or components of the virus, which may be used as a component of a vaccine. As used herein, the terms “inactivated”, “killed” or “KV” are used interchangeably.

The term “live vaccine” refers to a vaccine comprising a living, in particular, a living viral active component.

The optionally one or more pharmaceutically acceptable carriers or excipients, as mentioned herein include any and all solvents, dispersion media, coatings, adjuvants, stabilizing agents, diluents, preservatives, antibacterial and antifungal agents, isotonic agents, adsorption delaying agents, and the like. In some aspects, and especially those that include lyophilized immunogenic compositions, stabilizing agents include stabilizers for lyophilization or freeze-drying.

As used herein, the terms “vaccine” and “vaccine composition” are used interchangeably and in particular refer to a composition that will elicit a protective immune response in a subject that has been exposed to the composition.

As regards the term “for use in a method of reducing or preventing the clinical signs or disease associated with/related to/caused by natural IgM/IgA antibody deficiency (NAD) in a subject”, the same applies, mutatis mutandis, to the vaccine as has been outlined above for the first and second aspect of the present invention.

In a preferred embodiment, the vaccine for use in a method of reducing or preventing the clinical signs or disease associated with/related to/caused by natural IgM/IgA antibody deficiency (NAD) in a subject comprises a compound that induces human natural IgM and/or IgA antibody recognizing oxidized phospholipids and/or oxidation-specific epitopes.

As regards “human or humanized natural IgM and/or IgA antibody recognizing oxidized phospholipids and/or oxidation-specific epitopes”, the same applies, mutatis mutandis, to the vaccine as has been outlined above for the first and second aspect of the present invention.

In a preferred embodiment, the vaccine for use in a method of reducing or preventing the clinical signs or disease associated with/related to/caused by natural IgM/IgA antibody deficiency (NAD) in a subject is a Bacillus Calmette-Guérin (BCG) vaccine.

In a preferred embodiment, said Bacillus Calmette-Guérin (BCG) vaccine comprises an attenuated bacterium of Mycobacterium bovis.

In another preferred embodiment, the vaccine for use in a method of reducing or preventing the clinical signs or disease associated with/related to/caused by natural IgM/IgA antibody deficiency (NAD) in a subject is a pneumococcus tuberculosis vaccine.

In a preferred embodiment, the pneumococcus tuberculosis vaccine is the Pneumovax®23 vaccine, or the Prevenar® 3 vaccine.

In a preferred embodiment, said pneumococcus vaccine comprises polysaccharide epitopes from multiple pneumococcus strains.

In a preferred embodiment, the vaccine for use in a method of reducing or preventing the clinical signs or disease associated with/related to/caused by natural IgM/IgA antibody deficiency (NAD) in a subject is a vaccine that is capable of:
stimulating the production of natural IgM specific for oxidized phospholipids and/or oxidation-specific epitopes,
reducing the accumulation of free oxidized phospholipids, preferably in infected lungs,
reducing the accumulation of oxidized phospholipids and/or oxidation-specific epitopes on LDL, preferably in atherosclerotic lesions;
stimulating of IL-10 and/or TGFβ secretion, preferably by alveolar macrophages; and/or
reducing the accumulation of misfolded proteins such as oligomeric amyloid-β, preferably in brain tissues,
and/or neutralizing of pro-inflammatory cytokines.

As regards the “stimulating the production of natural IgM, reducing the accumulation of free oxidized phospholipids, preferably in infect lungs, stimulating of IL-10 and/or TGFβ secretion, preferably by alveolar macrophages; and/or neutralizing of pro-inflammatory cytokines”, the same applies, mutatis mutandis, to the vaccine as has been outlined above for the first and second aspect of the present invention.

In a preferred embodiment of the vaccine described above, said clinical signs or disease associated with natural IgM/IgA antibody deficiency (NAD) is an infectious disease.

More preferably, said infectious disease is a virus infectious disease.

Even more preferred, said viral infection disease is selected from the group consisting of infections by coronaviruses, preferably SARS-CoV, SARS-CoV-2, MERS); influenza viruses, parainfluenza viruses, respiratory syncytial viruses (RSV), rhinoviruses, adenoviruses, enteroviruses, human metapneumoviruses, herpesviruses, preferably HSV-1, HSV-2, VZV, EBV, HCMV, HHV-6, HHV-7, HHV-8.

In a more preferred embodiment, the vaccine for use in a method of reducing or preventing the clinical signs or disease associated with/related to/caused by natural IgM/IgA antibody deficiency (NAD) in a subject is a vaccine wherein the virus infection disease is COVID-19 caused by the β-Coronavirus SARS-CoV2.

As regards the “virus infection disease being COVID-19 caused by the β-Coronavirus SARS-CoV2”, the same applies, mutatis mutandis, to the vaccine as has been outlined above for the first and second aspect of the present invention.

In a preferred embodiment, the agent or compound, preferably the antigen, comprised in the vaccine of the present invention, may also be recombinantly or synthetically generated/synthesized.

Thus, in a preferred embodiment, the vaccine is a vaccine wherein at least some of the antigens contained therein are of recombinant origin.

Other aspects and advantages of the invention will be described in the following examples, which are given for purposes of illustration and not by way of limitation. Each publication, patent, patent application or other document cited in this application is hereby incorporated by reference in its entirety.

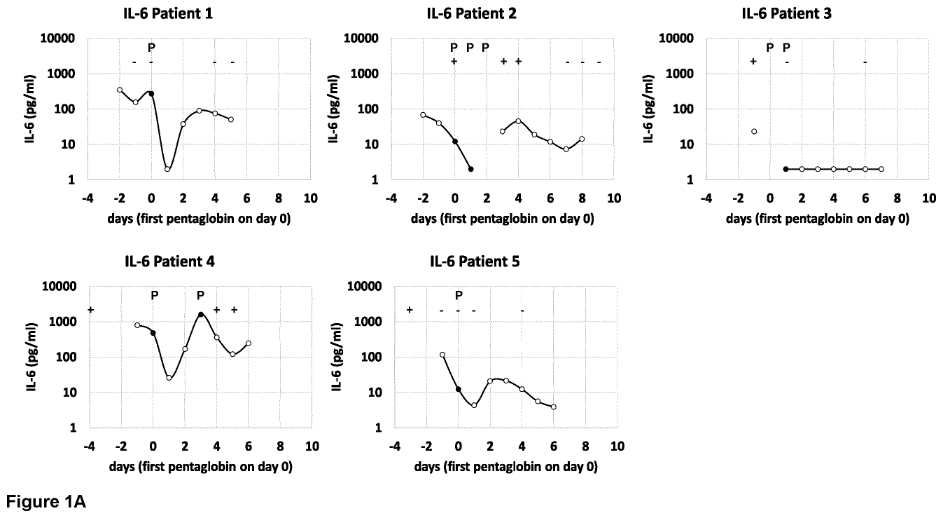

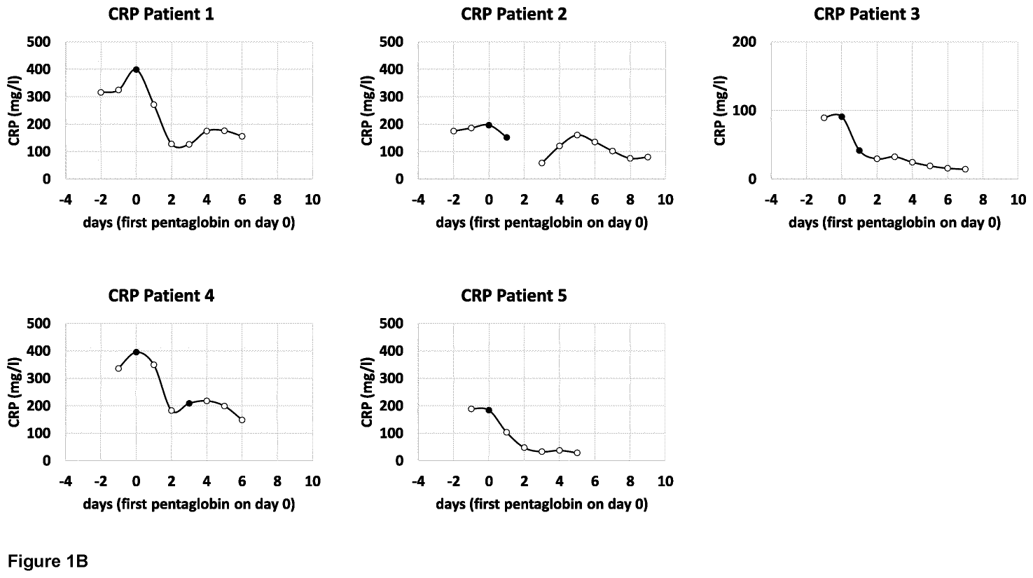

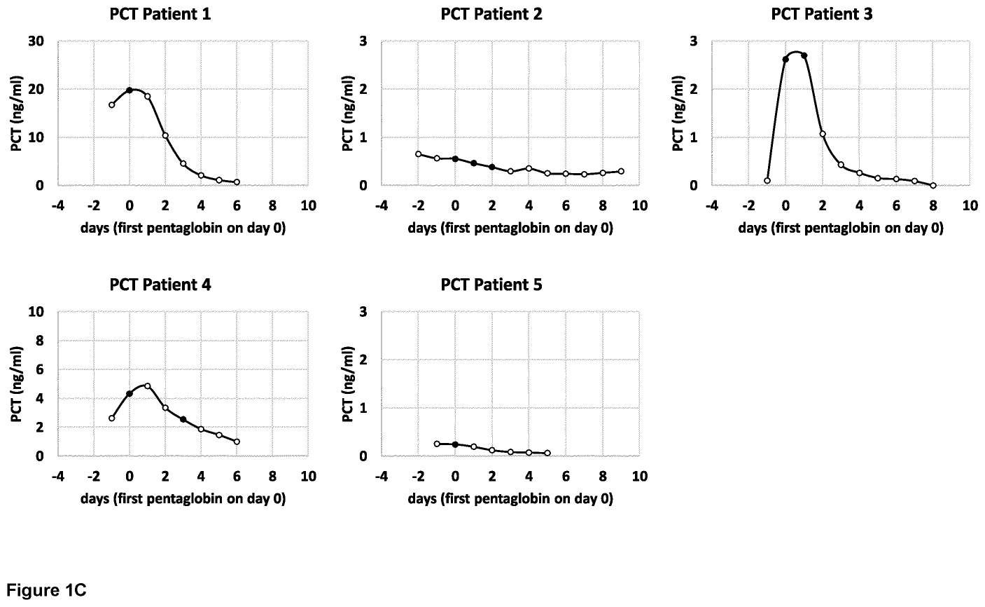

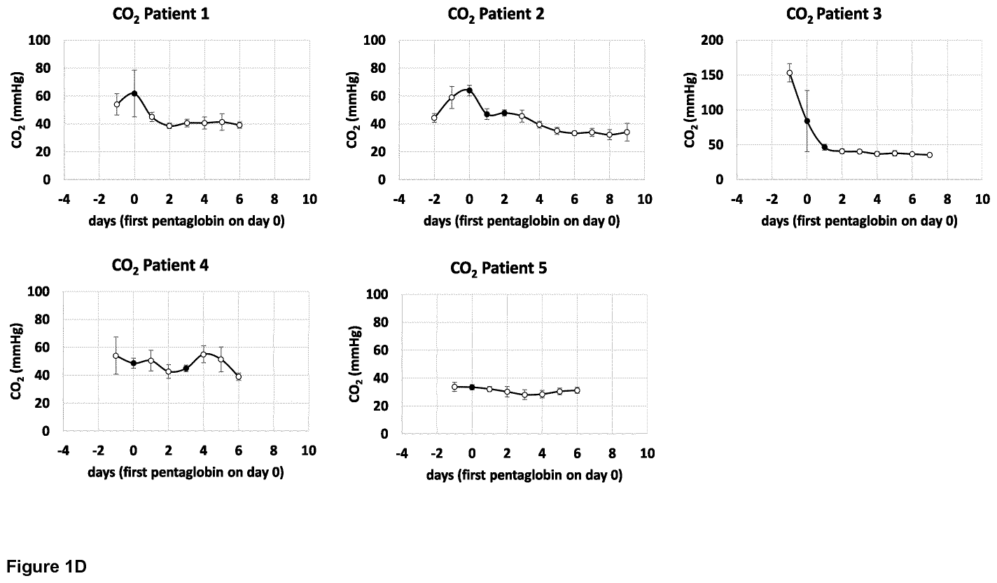

FIG. 1: Course of clinical parameters in COVID-19 patients treated with Pentaglobin®

Five patients presenting with deteriorating COVID-19 pneumonia were treated with Pentaglobin®“P” indicates days on which patients were treated with Pentaglobin® (closed circles). Clinical parameters IL-6 (A), CRP (B), PCT (C) or mean daily blood pCO₂ (error bars are standard deviations) (D) were measured over the time. Presence (+) or absence (−) of SARS-CoV-2 in bronchoalveolar lavages (BAL) was monitored.

FIG. 2: Oxidative stress responsible for excessive inflammation in COVID-19 patients.

Illustration of mechanisms leading to the formation of ROS and the accumulation of oxPL and OSE (FIG. 2A). Manipulation of these mechanisms, thereby counteracting the generation of OSE (FIG. 2B).

FIG. 3: Detection of anti-nuclear autoimmunantibodies in COVID-19 patient sera.

HD: Healthy donor serum; COV: COVID-19 serum; Positive Control (provided in Kallestad HEp2 Kit).

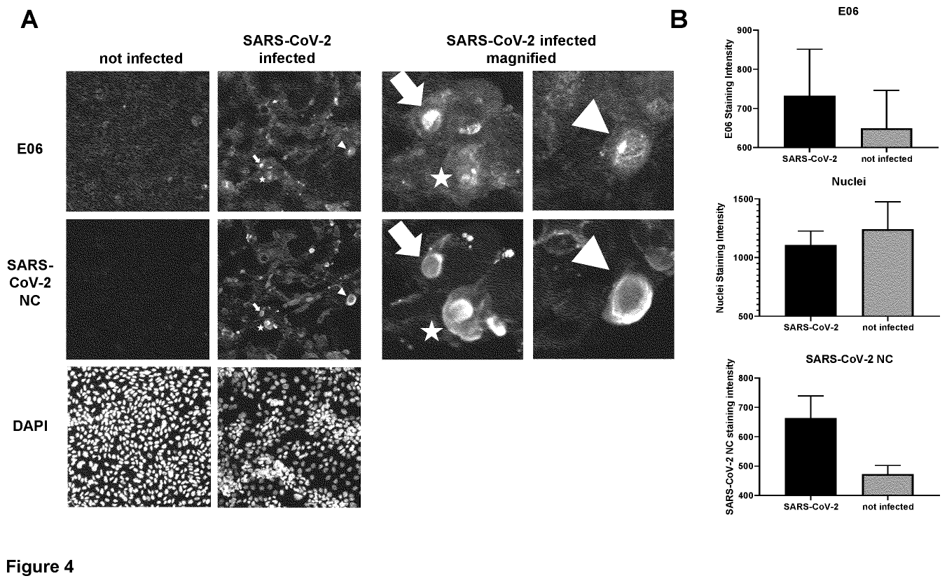

FIG. 4: SARS-CoV-2 infected lung cells have increased levels of oxPL. A) Lung adenocarcinoma cells (Calu-3) were infected for three days with SARS-CoV-2 and then subjected to immunostaining with the mouse monoclonal natural IgM E06 that detects the phosphorylcholine headgroup of oxPL. Cells were co-stained with anti-SARS-CoV-2 NC protein to demonstrate infection. DAPI staining allowed visualization of cell nuclei. Magnifications show co-stainings of rounding up apoptotic SARS-CoV-2 NC-positive cells with E06. B) Quantification of staining intensities of 4 independent images using ImageJ software.

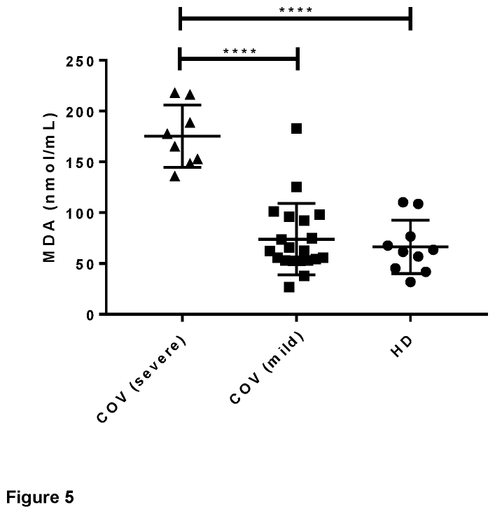

FIG. 5: Increased oxidative stress in sera from COVID-19 patients compared to healthy donors. Malondialdehyde (MDA) is one of the most used biomarkers for lipid peroxidation. The concentration of MDA was determined in sera derived from 8 hospitalized COVID-19 patients with severe disease, 20 outpatients with mild COVID-19, and 10 healthy donors by using a commercially available assay kit (Lipid Peroxidation (MDA) Assay Kit, Abcam) and according to manufactures instructions. Statistical significance was calculated using the Students-t Test. **** P<0.0001.

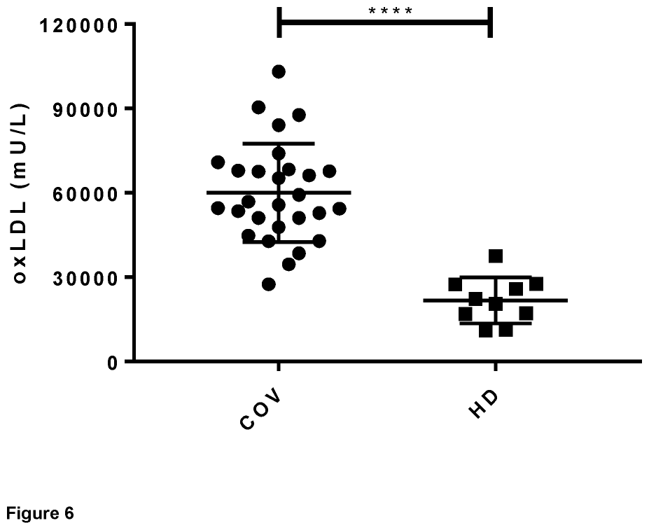

FIG. 6: Significantly elevated serum levels of oxLDL in COVID-19 patients. OxLDL levels were measured in sera derived from COVID-19 patients (COV, n=28) and healthy human donors (HD, n=10). OxLDL levels were measured using the oxidized LDL ELISA Kit (Cat. no. 10-1143-01, Mercodia) according to the manufacturer's instructions. Statistical significance was calculated using the Students-t Test. **** P<0.0001.

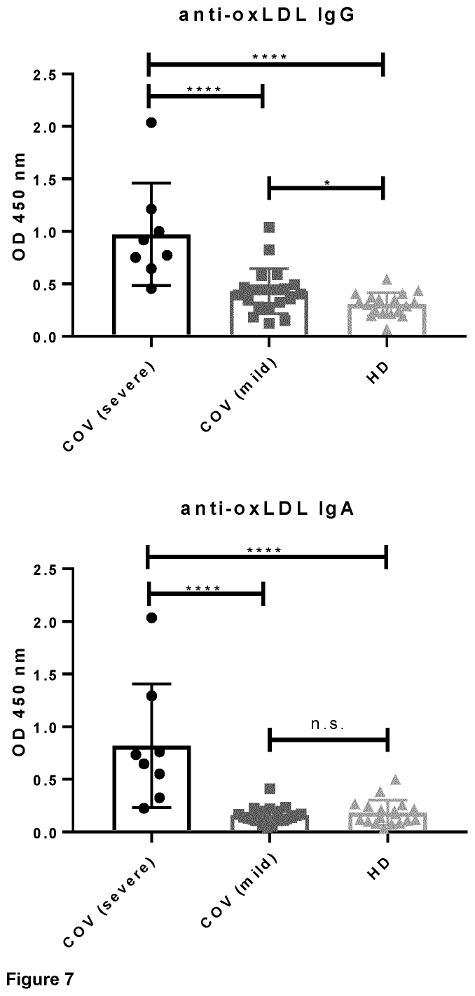

FIG. 7: Elevated anti-oxLDL IgG and IgA autoantibodies in sera from patients with severe COVID-19 compared to sera from healthy controls or mild COVID-19 cases. Plates were coated with oxLDL and coated plates were incubated with diluted sera (1:20). After washing, bound serum-derived anti-oxLDL antibodies were determined by adding anti-human IgG or anti-human IgA antibodies coupled to horse-reddish peroxidase. Readings of optical densities (OD) at 450 nm were performed after substrate addition. COV, serum from COVID-19 patients (severe disease, n=8; mild disease, n=20); HD, serum from healthy donors (n=10). Statistical significance was calculated using the Students-t Test. * P<0.05; **** P<0.0001.

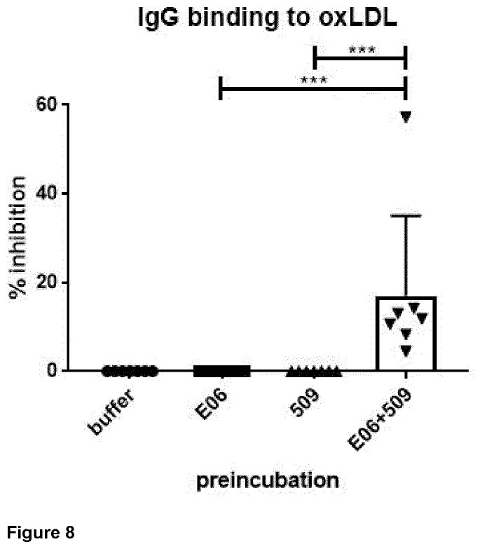

FIG. 8: OxPL-specific monoclonal antibodies compete with IgG from COVID-19 sera for binding to oxLDL. ELISA plates were coated with oxLDL and preincubated either with assay buffer or with mouse monoclonal antibodies E06, 509 or with a combination of both. Then serum samples from hospitalized COVID-19 patients with severe disease (n=8) were added to coated and preincubated wells and binding of IgG antibodies was detected using an HRP-conjugated anti-human IgG secondary antibody. IgG binding to oxLDL was determined by OD values at 450 nm and data are expressed as percent inhibition of IgG binding to oxLDL in wells preincubated with indicated monoclonal antibodies compared to wells preincubated with assay buffer. Each serum sample was tested in triplicates and statistical significance was calculated by one-tailed Mann-Whitney test. *** P<0.001.

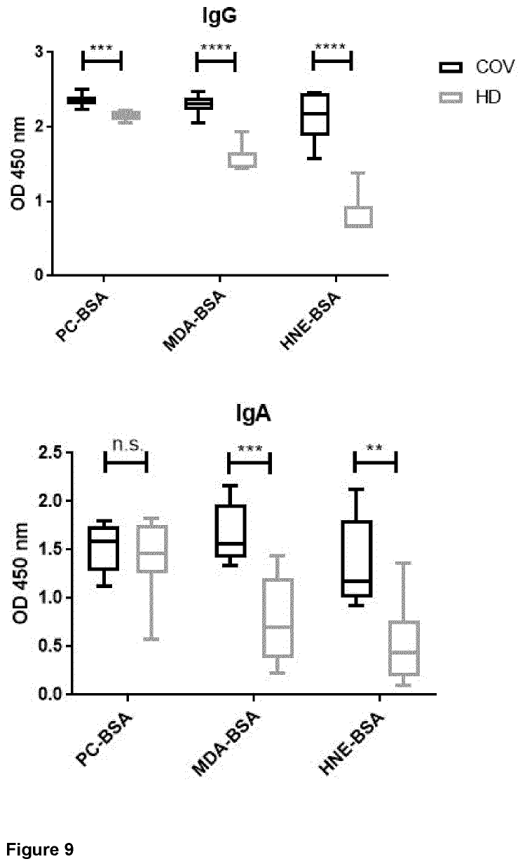

FIG. 9: COVID-19 sera contain elevated IgG and IgA antibodies to oxidation-specific epitopes. ELISA plates were coated with 10 μg/ml of the indicated antigens. Coated and blocked wells were incubated with human serum samples derived from hospitalized COVID-19 patients (COV; n=8) or healthy donors (HD; n=8) diluted 1:20, and bound antibodies were detected with HRP-conjugated and isotype-specific secondary antibodies. Each serum sample was tested in triplicate and antibody binding is expressed as optical density (OD) values. Statistical significance of antibody binding in COV vs. HD sera was calculated using the Students-t Test. ** P<0.01; *** P<0.001; **** P<0.0001.

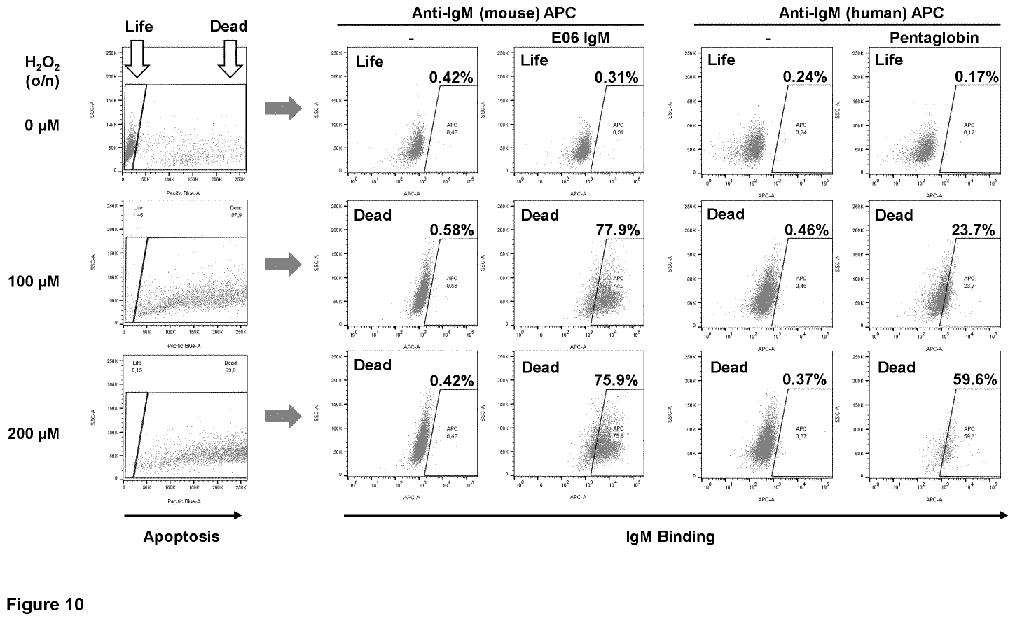

FIG. 10: An IgM fraction from Pentaglobin binds to apoptotic cells displaying oxPL. HEK293T cells were treated over night with different concentrations of H₂O₂ and subjected to immunostaining the next day with either the mouse monoclonal IgM E06 that binds to the phosphorylcholine headgroup of oxPL, or with Pentaglobin, and subsequently stained with species-specific APC-labelled anti-IgM antibodies. Control cells were incubated with the secondary antibodies only. Apoptotic cells were identified by Sytox positivity.

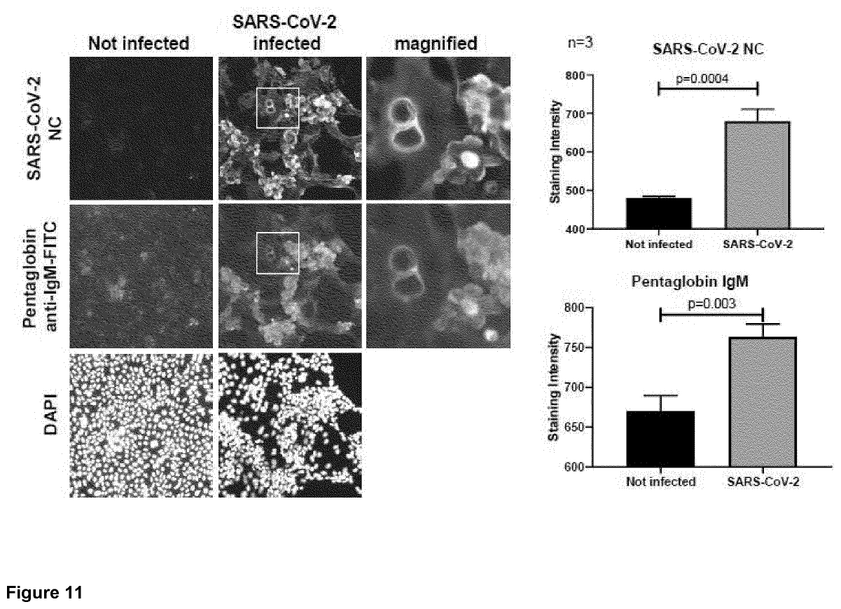

FIG. 11: An IgM fraction from Pentaglobin® binds to SARS-CoV-2 infected cells. Lung adenocarcinoma cells (Calu-3) were infected for three days with SARS-CoV-2 and then subjected to immunostaining with Pentaglobin and SARS-CoV-2 NC protein. FITC labeled anti-human IgM antibody was used to detect bound IgM fraction in Pentaglobin stained cells. DAPI staining allowed visualization of cell nuclei. Magnification shows staining signals of SARS-CoV-2 NC-positive cells with IgM from Pentaglobin. Quantification of mean values of staining intensities of 3 independent pictures using ImageJ software is shown on the right and error bars are standard deviations. Student t-test was used to determine statistical significance.

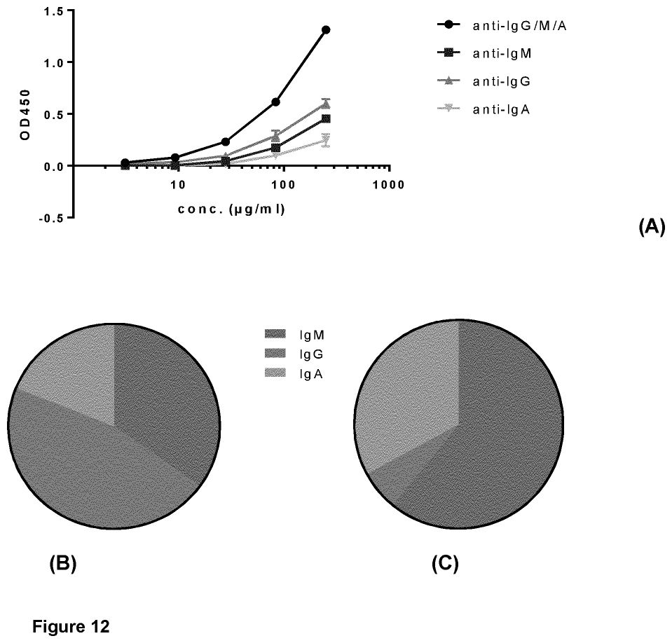

FIG. 12: Pentaglobin® contains antibodies that bind to oxLDL. ELISA plates were coated with oxLDL and incubated with indicated concentrations of Pentaglobin. Antibody binding to oxLDL was detected using HRP-conjugated isotype-specific antibodies. (A) Antibody binding is expressed as OD 450 nm values as a function of the concentration of Pentaglobin®. (B) Quantification of antibody isotypes within Pentaglobin that bound to oxLDL based on the ratio of OD450 values of individual isotypes to all isotypes. (C) Binding of individual isotypes to oxLDL was normalized to the concentration of the respective isotype within Pentaglobin.

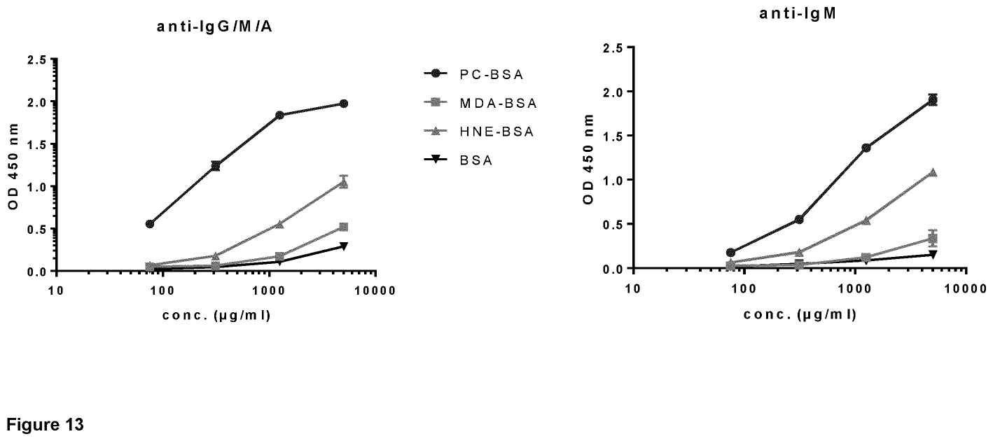

FIG. 13: Pentaglobin® contains antibodies that bind to oxidation-specific epitopes. ELISA plates were coated with Phosphorylcholine-BSA (PC-BSA), Malondialdehyde-BSA (MDA-BSA), 4-Hydroxynonenal-BSA (HNE-BSA) or unconjugated bovine serum albumin (BSA), and incubated with indicated concentrations of Pentaglobin®. Antibody binding to coated antigens was detected using HRP-conjugated isotype-specific antibodies recognizing the isotypes IgG, IgM and IgA (anti-IgG/M/A), or specifically IgM (anti-IgM). Antibody binding is expressed as OD 450 nm values as a function of the concentration of Pentaglobin®.

### EXAMPLES

### Example 1: Demonstration of the Binding of Generated nIgMs to Oxidized Lipids

Commercially available ELISAs (e.g. MDA-BSA coated plate ELISA (DEIACP15) or anti ox-LDL ELISA (DEIA081J) from Creative Diagnostics) are used to evaluate the binding of generated OSE-specific IgM and/or IgA antibodies to oxidized phospholipids. Avanti Lipid Snoopers® ELISA test strips coated with oxidized phosphatidycholine are used to analyze binding of IgM and/or IgA antibodies. IgM and/or IgA antibodies against oxidized cardiolipin or oxidized phosphatidylserine are measured using an ELISA method described in (Frostegard, Su et al., 2014, PLoS One, Vol. 9 (12)).

The binding to oxidized lipids is further investigated by inducing cell-apoptosis in cultured cells and staining with the generated OSE-specific IgM and/or IgA antibodies, Pentaglobin® or sera from BCG/Pneumovax-vaccinated individuals. Flow cytometry analysis is used to specifically look at apoptotic cells (e.g. marker annexin V) and binding of nIgMs is monitored by appropriate commercially available anti-IgM and anti-IgA secondary antibodies.

### Example 2: Demonstration of the Binding of OSE-Specific IgM and/or IgA Antibodies to Virus Infected Cells

Virus infection of cells leads to a plethora of events to ensure virus replication, including changes in the lipid composition on cellular membranes. A variety of viruses for example induces flipping of phosphatidyl-serines towards the exterior of the cell membrane, where they are prone for oxidation, normally a process that takes place during cell apoptosis and reflecting an ‘eat-me’ signal for phagocytes. The induced change in the lipid composition on cell membranes is incorporated into viral membranes during virus budding. Hence, some viruses use this as a mask to improve uptake by phagocytic cells, that recognize the virus as an apoptotic body, which can serve for better infection and virus spread. The changes in the lipid composition in viral membranes, but also viral surface glycoproteins directly may influence nIgM binding.

The binding of produced monoclonal OSE-specific IgM and/or IgA antibodies to Herpes Simplex Virus Type 1 or 2 infected cells or to cells expressing viral surface glycoproteins (e.g. Severe Acute Respiratory Syndrome Coronavirus 2 (SARS-CoV-2) glycoprotein S, Human Immunodeficiency virus (HIV-1) Env, Vesicular stomatitis virus (VSV) glycoprotein G, HSV-1/2 glycoproteins gB, gD, gH, gL, gI or gE) is tested. In these assays, affinity of binding (Kd) is determined for cells that express the respective viral glycoproteins and cells that do not express the protein as negative control.

### Example 3: The Demonstration of the Direct Antiviral Activity of Pentaglobin®, OSE-Specific IgM and/or IgA Antibodies and Serum from BCG/Pneumovax-Vaccinated Individuals In Vitro and In Vivo

Different in vitro and in vivo models are used to address the direct antiviral effects of Pentaglobin®, monoclonal OSE-specific IgM and/or IgA antibodies and serum from BCG/Pneumovax-vaccinated individuals on virus infection and spread.

Prototype viruses from diverse virus families are investigated. These include Herpesviridae (HSV-1/2), Rabdoviridae (recombinant VSV with GFP reporter, rVSVdeltaG-GFP), as well as Murine Leukemia Virus (MLV) and HIV-1-based vectors pseudotyped with different gylcoproteins including VSV-G, HIV-1 Env, or SARS-CoV-2 glycoprotein S.

It is conceivable that enveloped viruses contain oxidized phospholipids and OSE in their membrane that can be bound by OSE-specific IgM and/or IgA antibodies. Direct effects on cell-free virus infection are tested by incubating serial dilutions of pre-BCG/Pneumovax-vaccination and post-vaccination serum, monoclonal OSE-specific IgM and/or IgA antibodies or Pentaglobin® with viruses or viral vectors and subsequent infection of appropriate target cell lines, e.g. the African green monkey cell line Vero or human embryonic kidney cell line HEK293T. Depending on the virus or vector that is used, infection is measured either by plaque formation (HSV-1 or HSV-2) or by reporter genes delivery through virus or viral vector infection (e.g. GFP or Luciferase).

For HSV-1 and HSV-2 effects of pre-/and post-BCG/Pneumovax vaccination sera, Pentaglobin® and monoclonal OSE-specific IgM and/or IgA antibodies on virus cell-to-cell spread are measured by infecting target cells with virus and then applying serial dilutions of the aforementioned drugs containing OSE-specific IgM and/or IgA antibodies.

Immunodeficient as well as immunocompetent mice are infected intravaginally with HSV-1 or HSV-2 and inject either before infection (prophylactic approach) or after infection (therapeutic approach) different doses of monoclonal OSE-specific IgM and/or IgA antibodies, Pentaglobin® or sera derived from BCG/Pneumovax-vaccinated individuals.

Survival and lesion development are monitored over time. Viral replication is measured via quantitative PCR. In these experiments Pentaglobin® serves as positive control, since due to the high seroprevalence of HSV it will likely contain virus-neutralizing IgGs.

Natural IgMs are also known to protect from primary cutaneous infections with HSV-1 (Deshpande, Kumaraguru et al., 2000, Cell Immunol, Vol. 202 (2)). It is investigated whether intravenous prophylactic or therapeutic injection of OSE-specific IgM and/or IgA antibodies or sera from BCG/Pneumovax-vaccinated individuals protects from cutaneous lesions induced by HSV-1 infection.

It is also tested whether topical treatment with these OSE-specific IgM and/or IgA antibody-containing compounds prevents lesion development after primary cutaneous HSV-1 infection. Since HSV-1 seroprevalence in the human population is approximately 80% we are exclusively using sera from HSV-1/2 seronegative (naïve) individuals.

### Example 4: Analyzing of the Binding of nIgMs to Specific Leukocyte Surface Proteins to Prevent Induction of Proinflammatory Responses

(Lobo, Schlegel et al., 2008, J Immunol, Vol. 180 (3)) (Lobo, 2016, Front Immunol, Vol. 7)) demonstrated that IgM-ALA (leukocyte-binding nIgMs) have a positive role in recipients of heart and kidney transplants. Low or no levels of IgM-ALA was associated with increased inflammation, host vs. graft disease and transplant loss. It was demonstrated that IgM-ALA bind to (i) certain co-stimulatory receptors, that is, CD4, CD86, CD40, and PD1 and (ii) chemokine receptors. However, these polyreactive IgM-ALA autoantibodies manifest some form of specificity as they do not randomly bind to glycoproteins on other cell receptors, that is, CD8, CD80, CD40L, PDL1, CD28, CD1d, and HLA receptors. It was also shown that IgM-ALA bind to TcR, CD3, and CD45.

It is investigated by ELISAs and by flow cytometric assays whether identified and selected monoclonal nIgMs bind aforementioned co-stimulatory receptors of leukocytes. Prevention of the induction of proinflammatory responses by these nIgMs can be monitored by incubation of primary blood monocytes or purified leukocyte populations with nIgMs under stimulating conditions followed by multiplex analysis of secreted cytokines using established flow cytometry-based methods (Biolegend).

### Example 5: Anti-Inflammatory Effects of Pentaglobin® or Monoclonal OSE-Specific IgM and/or IgA Antibodies or Sera from BCG/Pneumovax-Vaccinated Individuals on Acute Respiratory Disease Syndrome (ARDS) In Vivo

SARS-CoV-2 infection causes the development of Coronavirus disease (COVID-19), which can present with subclinical or mild symptoms as well as with severe symptoms reflecting an acute respiratory disease syndrome (ARDS). Two preclinical mouse models of ARDS that arise from direct lung injury have been described (D'Alessio, 2018, Methods Mol Biol, Vol. 1809)). These use intratracheal instillation of LPS (sterile inflammation) or Streptococcus pneumoniae to mimic human pneumonia. The models are chosen because they are highly reproducible, elicit robust neutrophilic alveolitis and disruption of the alveolar-capillary membrane, are easily titratable (degrees of pulmonary inflammation), and allow for evaluation of both the early and resolution phases of acute lung injury (ALI).

ARDS mice are treated with Pentaglobin® injection, or injection with monoclonal nIgMs or sera from BCG/Pneumovax-vaccinated individuals or non-vaccinated control sera. The response to treatment, i.e. alleviation of ARDS is monitored.

### Example 6: Generation of OSE-Specific IgM and/or IgA Antibodies Libraries from BCG/Pneumovax-Vaccinated SARS-CoV-2 Seropositive Individuals with Mild/No Symptoms During SARS-CoV-2 Infection

It is demonstrated that BCG/Pneumovax-vaccinated individuals have a benefit in fighting severe infections with immunologically unseen pathogens due to increased levels of natural antibodies.

It was shown, that the repertoire of natural IgM changes with age in both level of expression as well as diversity (Rodriguez-Zhurbenko, Quach et al., 2019, Front Immunol, Vol. 10)). The conclusion from this observation is that the nIgM repertoire from young individuals may have enhanced anti-pathogenic characteristics.

In order to test this, natural IgM and/or IgA antibody phage-display libraries are generated from young BCG/Pneumovax-vaccinated SARS-CoV-2 seropositive individuals that showed only mild or no symptoms during the SARS-CoV-2 infection phase. From these natural antibody libraries several monoclonal IgM and/or IgA antibodies are cloned that show characteristic low affinity polyreactive binding properties to natural antibody-specific antigens and are able to bind to viral glycoproteins and show virus-neutralizing activities. The monoclonal IgM and/or IgA antibodies are tested individually and in combination (pools) to test for enhanced virus-neutralizing activity when used as a pool.

### Example 7. Population-Wide Analysis of OSE-Specific IgM and/or IgA Antibody Serum Concentration and Correlation with BCG/Pneumovax-Vaccination Status

To demonstrate that BCG/Pneumovax vaccine induces higher levels of protecting nIgMs nIgM concentrations are analyzed and compared in sera from individuals that were not BCG and/or Pneumovax-vaccinated with sera from BCG and/or Pneumovax-vaccinated individuals. OSE-specific IgM and/or IgA serum levels are determined by ELISA methods described above. Pooled sera from vaccinated and not-vaccinated individuals are compared for virus-neutralizing activities in the viral assays described above.

### Example 8. Defining the In Vivo Effects of Pentaglobin®, Monoclonal OSE-Specific IgM and/or IgA and Sera from BCG-Pneumovax-Vaccinated Individuals on Atherosclerosis

The two most frequently used models of mouse atherosclerosis are the apoE−/− model and the Idlr−/− model. It is tested whether injection of Pentaglobin®, monoclonal nIgMs or sera from BCG/Pneumovax-vaccinated individuals reduces atherosclerosis in these two models.

### Example 9. SARS-CoV-2 Infected Lung Cells have Increased Levels of oxPL

As explained above, pathogens such as SARS-CoV-2, SARS-CoV or H5N1 influenza virus trigger increased rates of lipid peroxidation in cellular membranes, which is the production of oxidized phospholipids (oxPL) that occurs as a result of oxidative damage. It is postulated that OxPL is generated by SARS-CoV-2 infected cells as a result of cellular stress responses.

In fact, the accumulation of oxPL was detected on the surface of SARS-CoV-2 infected lung adenocarcinoma cells (Calu-3) as determined by stronger staining intensities of infected cells with the murine natural IgM E06, which detects the phosphorylcholine headgroup of oxPL, but not of native PL (FIG. 4). These data suggest that SARS-CoV-2 infected cells represent an important source of oxPL that potentially accumulate to proinflammatory concentrations when not cleared efficiently, e.g. by natural IgM antibodies.

### Example 10. Increased Oxidative Stress in Sera from COVID-19 Patients

Lipids containing polyunsaturated fatty acids are particularly susceptible to an oxidative attack, typically by reactive oxygen species (ROS), resulting in a chain reaction with the production of oxPL and end products such as malondialdehyde (MDA) that additionally contribute to the pathology of pathogen-induced inflammation in the infected tissue. OxPL and aldehydes such as MDA are not exclusively localized to the tissue where they were formed by ROS and cellular stress responses, but can also enter the circulation after they are released by cells under oxidative stress.

Since an increased formation of oxPL by SARS-CoV-2 infected cells was detected (see above), it is postulated that degradation products such as MDA may be elevated in sera of COVID-19 patients.

Indeed, significantly elevated concentrations of MDA were found in the sera derived from hospitalized COVID-19 patients and high MDA serum concentrations discriminated patients suffering from severe disease from patients with mild disease or healthy donors (FIG. 5). It is postulated that the high MDA serum level in severe COVID-19 patients results from defective clearance mechanisms involved in the neutralization and degradation of oxPL and OSE, resulting in the accumulation of oxPL and OSE to concentrations high enough to induce proinflammatory immune responses that contribute to the hyperinflammation state observed in severe COVID-19 patients.

### Example 11. Significantly Elevated Serum Levels of oxLDL in COVID-19 Patients

Human low-density lipoprotein (LDL) is one of the key lipid-protein complexes in blood and is a crucial component of metabolism responsible for the transport of lipids throughout the body. OxPL and aldehydes such as MDA in circulation can induce oxidative modifications of circulating LDL and other lipoproteins (Parthasarathy et al., 2010, Methods Mol Biol, Vol. 610 (403)).

Oxidation of LDL is a complex process during which both the lipids and proteins undergo oxidative changes and form complex products. For example, the peroxidised lipids decompose generating aldehydes such as MDA that covalently modify amino groups of lysine residues in apolipoprotein-B100 (apoB-100) of LDL. This not only generates Schiff's bases that modify charges on the amino acids, but also results in proteolysis of the apoB-100 protein as well as in both intra- and intermolecular crosslinks between proteolyzed apoB-100, resulting in excessive alteration of the protein composition and structure. Therefore, as opposed to native LDL, oxidized LDL (oxLDL) particles possess a variety of novel antigenic determinants that are recognized by receptors of innate and adaptive immunity, and cells of the vascular wall, thereby playing a key pathogenic role in cardiovascular diseases (CVD). In CVD, the proinflammatory functions of oxLDL are mediated by dendritic cells and macrophages that bind oxLDL with high affinity via scavenger receptors, leading to uncontrolled uptake of oxLDL and conversion of macrophages to foam cells, the defining characteristic of fatty streak and atherosclerotic lesions. Also, T cell activation has been linked to modified LDL since peptides derived from oxLDL have been shown to be recognized by T cells (Stemme et al., 1995, Proc Natl Acad Sci, Vol. 92 (3893)).

The surprising finding of the present invention that COVID-19 patients with severe disease contain high concentrations of MDA in their serum (FIG. 5) indicates that circulating lipoproteins such as LDL can be oxidatively modified by released aldehydes from infected SARS-CoV-2 cells.

In line with this, significantly elevated levels of oxLDL were detected in sera derived from COVID-19 patients as compared to sera from healthy donors (FIG. 6). Interestingly, the mouse monoclonal antibody 4E6 used to detect oxLDL in this assay, binds to a conformational epitope in the apoB-100 moiety of LDL that is generated as a consequence of substitution of at least 60 lysine residues of apoB-100 with aldehydes. This number of substituted lysines corresponds to the minimal number required for scavenger receptor-mediated uptake of oxLDL by macrophages.

Thus, the present results demonstrate three novel and surprising findings:
1) high amounts of oxPL- and OSE-bearing structures such as SARS-CoV-2-infected cells and cellular debris are formed in lungs and possibly other infected tissues in COVID-19 patients;
2) aldehydes such as MDA are released into serum of COVID-19 patients, e.g., by SARS-CoV-2 infected lung cells, where they induce oxidative modifications to circulating lipoproteins such as LDL; and
3) circulating oxidized LDL particles in COVID-19 patients show requirements for high-affinity binding to scavenger receptors expressed by innate immune cells and, therefore, possess the potential to trigger systemic proinflammatory responses in a similar way as described for CVD when not cleared efficiently.

### Example 12. Elevated Anti-oxLDL IgG and IgA Autoantibodies in Sera from Patients with Severe COVID-19

It is known from diseases associated with increased oxLDL levels, such as atherosclerosis and other CVD, diabetes mellitus, systemic lupus erythematosus or rheumatoid arthritis, that up to 90% of oxLDL are complexed by autoantibodies generating oxLDL-immune complexes, which levels often correlate with severity of the disease.

The autoantibodies that are complexed with oxLDL under disease conditions are predominantly of the IgG1 and IgG3 isotypes, which are both proartherogenic and can exert proinflammatory responses through their interaction with Fc-gamma receptors expressed by innate immune cells such as macrophages (Mironova et al.; Arterioscler Thromb Vasc Biol.; 1996 February; 16(2):222-9) (Virella et al., 2003, Clin Diagn Lab Immunol, Vol. 10 (499)). Thereby, oxLDL-IgG immune complexes induce stronger proinflammatory responses as compared to free oxLDL because the immune complexes engage Fc-gamma receptors in addition to scavenger receptors. Fc-gamma receptor-mediated NLRP3 inflammasome activation contributes to the secretion of proinflammatory cytokines, e.g. IL-1b and IL-6, from innate immune cells in response to oxLDL-IgG immune complexes (Rhoads et. al; J Immunol; 2017 Mar. 1; 198(5):2105-2114).

Since COVID-19 patients with severe disease surprisingly showed highly elevated level of oxLDL in their sera compared to patients with mild disease or healthy donors, it was tested if these patients also contained increased titers of anti-oxLDL IgG autoantibodies that potentially form proinflammatory immune complexes.

In full support of the concept presented herein, significantly elevated levels of anti-oxLDL IgG autoantibodies in sera from COVID-19 patients compared to sera from healthy donors, and these levels correlated with the severity of the disease (FIG. 7).

Since a fraction of serum IgA antibodies, particularly of the IgA2 subtype, can exert proinflammatory responses via binding of IgA2-containing immune complexes to macrophages and neutrophils expressing the Fc-alpha receptor, anti-oxLDL IgA autoantibody levels were also compared between COVID-19 patients with severe disease, with mild disease and healthy donors. Interestingly, it has been found that anti-oxLDL IgA autoantibody levels were specifically and significantly increased in patients with severe compared to mild forms of COVID-19 or healthy donors (FIG. 7).

These surprising findings support a role for pathogenic oxLDL-IgG and oxLDL-IgA immune complexes in severe COVID-19 patients, where they trigger systemic hyperinflammation responses.

### Example 13. OxPL-Specific Monoclonal Antibodies Compete with IgG from COVID-19 Sera for Binding to oxLDL

OxLDL particles display a large variety of immunogenic determinants that have not yet been defined in detail and that can be bound by antibodies.

To show that anti-oxLDL antibodies in sera from COVID-19 patients indeed bind to oxPL, an oxPL-masking assay was performed using two monoclonal mouse antibodies that bind to distinct classes of oxPL:

IgM antibody E06 binds to the phosphorylcholine headgroup exposed by oxidized phosphatidylcholine (oxPC), whereas IgM antibody 509 binds to oxidized phosphatidylethanolamine (oxPE) but not to its native non-oxidized counterpart (Bochkov et al., 2016, Biomark Med., Vol. 10 (8), 797-810).

ELISA plates were coated with oxLDL and the coated wells were preincubated with these monoclonal antibodies alone or in combination to block oxPC and/or oxPE exposed by oxLDL. Then the COVID-19 serum samples were added and IgG binding to oxLDL was detected by HRP-conjugated anti-human IgG secondary antibody.

The data revealed that blocking oxPC or oxPE alone on oxLDL by preincubation with one of these two monoclonal antibodies did not lead to detectable inhibition of IgG binding to oxLDL.

However, when both classes of oxPL were blocked simultaneously, a significant inhibition of binding of IgG antibodies in COVID-19 sera to oxLDL particles by ˜20% on average was observed (FIG. 8). These data suggest that anti-oxLDL IgG autoantibodies in COVID-19 patients indeed contain specificities toward oxPL and likely contain additional clones that recognize OSE other than oxPC and oxPE.

### Example 14. COVID-19 Sera Contain Elevated IgG and IgA Antibodies to Oxidation-Specific Epitopes

OxPL such as oxPC and oxPE can be predominantly found on OxLDL particles in the early phase of oxidation, whereas end products of lipid peroxidation such as Malondialdehyde or 4-Hydroxynonenal dominate on LDL particles with an advanced oxidation state.

To show that anti-oxLDL antibodies in sera from COVID-19 patients bind to different types of OSE exposed by oxLDL particles, ELISA plates were coated with phosphorylcholine (PC), Malondialdehyde (MDA) and 4-Hydroxynonenal (HNE), representing early and late-stage oxidation-specific epitopes. Coated wells were incubated with sera derived from hospitalized COVID-19 patients with severe disease or with sera from healthy donors, and antibody binding was detected by isotype-specific HRP-conjugated secondary antibodies.

It was found that sera from healthy donors contained IgG and IgA antibodies that bound to PC, whereas levels of anti-MDA and anti-HNE IgG and IgA antibodies were low or undetectable, respectively (FIG. 9).

Since PC is the immunodominant determinant of pneumococcal cell-wall polysaccharides, anti-PC IgG and IgA antibodies in healthy donors may represent previous pneumococcal infection or vaccination histories. However, and in sharp contrast, highly significantly elevated levels of IgG and IgA antibodies were detected that bound to MDA and HNE in sera from severe COVID-19 patients compared to sera from healthy donors (FIG. 9), indicating that a major fraction of anti-oxLDL IgG and IgA autoantibodies in sera from COVID-19 patients indeed bind to oxidation-derived epitopes exposed by LDL.

This novel finding supports the following mechanism that contributes to the pathogenesis of severe COVID-19:
1) oxPL and OSE are formed in COVID-19 patients;
2) oxPL and OSE may not be cleared efficiently in a population of COVID-19 patients, e.g., because of a natural antibody deficiency, leading to accumulation of oxPL and OSE in SARS-CoV-2 infected cells, apoptotic cells and lipoproteins, which then trigger autoimmune responses and generation of IgG and IgA autoantibodies;
3) anti-oxPL and OSE IgG and IgA autoantibodies bind to oxPL- and OSE-bearing structures, e.g., on LDL particles or infected cells, and mediate proinflammatory immune responses by simultaneously engaging scavenger receptors and Fc-receptors expressed by innate immune cells, thereby driving the hyperinflammation state observed in severely ill COVID-19 patients.

### Example 15. An IgM Fraction from Pentaglobin Binds to Apoptotic Cells Displaying oxPL

As shown above, COVID-19 patients develop oxidative stress and an increased exposure of oxPL in the membranes of SARS-CoV-2 infected cells and circulating lipoprotein particles was found. These structures possess the ability to induce IgG and IgA autoantibody responses and it is postulated that they contribute to the pathogenesis of severe COVID-19 if not cleared efficiently.

In mice, natural IgM antibodies and other soluble pattern recognition receptors (PRRs) of innate immunity bind to oxPL- and OSE-bearing structures and thereby facilitate their safe and anti-inflammatory clearance.

Therefore, the novel concept is herewith supported that treatment of severely ill COVID-19 patients with oxPL-specific natural IgM antibodies neutralize the proinflammatory effects of oxPL, facilitate their safe clearance and thereby prevents the induction of destructive autoimmune responses.

Pentaglobin is a human immunoglobulin infusion preparation enriched for IgM and IgA antibodies and is approved to treat patients with severe bacterial infections and sepsis, and immunodeficient patients that lack endogenous immunoglobulins. Since the immunoglobulins in Pentaglobin constitute of pooled serum antibodies obtained from thousands of healthy human donors, it has been found herein that particularly the IgM pool, and to a lesser extend the IgA and IgG fractions, of Pentaglobin contain natural antibodies recognizing different types of oxPL and OSE.

To test for the presence of oxPL-specific IgM antibodies, Pentaglobin was used to stain human cells under oxidative stress exposing different types of oxPL and OSE in their plasma membrane and IgM binding was detected by fluorescently labelled secondary antibodies.

Oxidative stress was induced by incubation of cells with different concentrations of H₂O₂, an agent that potently initiates the lipid peroxidation reaction, and formation of oxPL was monitored by staining of treated cells with the mouse natural IgM E06 recognizing the phosphorylcholine headgroup exposed by oxPL. Indeed, most H₂O₂-treated cells stained positive with E06 and this was dependent on induction of apoptosis, indicating that treated cells displayed a huge amount of oxPL on their surface (FIG. 10). When Pentaglobin was used for staining, it was found that a significant fraction of cells treated with 100 μM H₂O₂ were bound by an IgM fraction within the formulation, and IgM binding positively correlated with increased rates of apoptosis and, hence, the presentation of oxPL. Thus, these results show the novel finding that Pentaglobin indeed contains a fraction of natural IgM antibodies that bind to oxPL exposed by apoptotic cells.

### Example 16. An IgM Fraction from Pentaglobin® Binds to SARS-CoV-2 Infected Cells

As shown above, it has been found that lung adenocarcinoma cells (Calu-3) infected with SARS-CoV-2 display large amounts of oxPL in their membranes that were bound by the mouse natural IgM antibody E06 (FIG. 4). Moreover, as also shown above, antibodies within the Pentaglobin formulation that bound to oxPL exposed by cells under oxidative stress have been found (FIG. 10).

To test if Pentaglobin contains antibodies that bind to SARS-CoV-2 infected lung cells, e.g., to oxPL exposed in the plasma membrane of infected cells, infected Calu-3 cells were stained with Pentaglobin and IgM binding was detected by incubation with fluorescently labelled anti-human IgM secondary antibody. Indeed, the data suggest that Pentaglobin contains an IgM fraction that can bind to infected cells and that most of these antibodies likely bind to oxPL presented on the plasma membrane (FIG. 11).

### Example 17. Pentaglobin® Contains Antibodies that Bind to oxLDL

OxPL are not exclusively exposed by membranes of apoptotic cells but can be present also on circulating lipoproteins such as LDL, where they constitute the major pathogenic component of oxLDL.

In fact, high amounts of oxPL were found in the plasma membrane of SARS-CoV-2 infected cells and in circulating oxLDL in sera from COVID-19 patients. Since it has surprisingly been found herein that Pentaglobin contains natural IgM antibodies that bind to apoptotic cells displaying high amounts of oxPL, it was tested whether Pentaglobin contains antibodies that also bind to oxLDL. To test this, ELISA plates were coated with oxLDL, the coated wells were incubated with different concentrations of Pentaglobin®, and antibody binding was detected by labelled isotype-specific secondary antibodies.

In fact, it has been found that Pentaglobin contains antibodies that bind to oxLDL in a concentration-dependent manner (FIG. 12A). When isotype-specific secondary antibodies were used for detection, it was found that most of oxLDL-binding antibodies within the Pentaglobin formulation were IgG (˜45%), followed by IgM (˜35%) and IgA (˜19%) (FIG. 12B). However, given that immunoglobulins in Pentaglobin® are composed of 78% IgG, 6% IgM and 6% IgA isotypes, the ratio of oxLDL binding isotypes was normalized to their ratio present in the formulation and it was found that the majority of oxLDL binding antibodies are indeed of the IgM isotype (˜61%), followed by IgA (˜33%) and IgG (˜6%) (FIG. 12C).

For uninfected mice it was shown that ˜80% of the IgM pool and ˜50% of serum IgA are derived from B1 cells, hence IgM and IgA represent the most common isotypes of natural antibodies (Meyer-Bahlburg, 2015, Ann N Y Acad Sci, Vol. 1362, 122-31). Therefore, this supports that most of oxLDL-binding IgM and IgA antibodies within Pentaglobin represent human natural antibodies.

### Example 18. Pentaglobin® Contains Antibodies that Bind to Oxidation-Specific Epitopes

To show that anti-oxLDL antibodies in Pentaglobin indeed bind to OSE exposed by oxidized LDL particles, ELISA plates were coated with different classes of OSE including Phosphorylcholine (PC), Malondialdehyde (MDA) and 4-Hydroxynonenal (HNE), which are well-described targets for natural antibodies.

Coated wells were incubated with 4 consecutive dilutions of Pentaglobin and antibody binding was detected by HRP-conjugated secondary antibodies. The results showed that Pentaglobin® indeed contains antibodies that bind to all classes of OSE tested, and that anti-PC antibodies constitute the most prominent OSE-binding fraction, followed by anti-HNE and lower level of anti-MDA antibodies.

Interestingly, when an IgM-specific secondary antibody was used to specifically detect IgM binding, we found a similar binding pattern to all classes of OSE tested, indicating that OSE-binding antibodies in Pentaglobin® primarily belong to the IgM pool (FIG. 13). High anti-PC IgM titers are typically found in sera from healthy human donors and most likely result from previous bacterial infections or immunization with pneumococcal extracts (e.g. Pneumovaxx23) (Nishinarita, 1990, Med. Microbiol. Immunol., Vol. 179, 205-214).

In support of this, it has been shown that pneumococcal immunization of mice induced high level of PC-specific natural IgM antibodies, which conferred protection from atherosclerosis due to molecular mimicry between S. pneumoniae and oxLDL (Binder, 2003, Nat. Med., Vol. 9, 736-43).

Taken together, these results show that Pentaglobin contains natural antibodies primarily of the IgM isotype that bind to different classes of OSE and we suggest that these antibodies may confer protection from oxPL-induced proinflammatory responses in severe COVID-19 patients.

### Example 19. Pentaglobin® Contains Antibodies that Block Binding of IgG and IgA from COVID-19 Sera to oxLDL

The above findings indicate that the severity of COVID-19 is accompanied by the development of oxPL- and OSE-specific IgG and IgA autoantibodies that form immune complexes with oxidatively modified particles such as circulating lipoproteins, which represent potent drivers of the hyperinflammation state observed in severely ill COVID-19 patients.

The above data support the novel concept that neutralization of oxPL and OSE by natural IgM antibodies neutralize their proinflammatory potential in COVID-19 patients by multiple mechanisms:
1) natural IgM block binding of oxidatively modified particles such as oxLDL to scavenger receptors on innate immune cells;
2) natural IgM block binding of IgG and IgA autoantibodies to oxidatively modified particles such as oxLDL and thereby prevent formation of proinflammatory IgG- and IgA-containing immune complexes;
3) natural IgM facilitate the safe and anti-inflammatory clearance of structures exposing oxPL such as cellular debris and thereby prevent the accumulation of oxPL and OSE and the development of IgG and IgA autoantibodies.

The above surprising findings that neutralization of two classes of oxPL on oxLDL by monoclonal mouse IgM antibodies significantly inhibited binding of IgG autoantibodies from COVID-19 sera to oxLDL, and that Pentaglobin contains a significant fraction of oxPL-specific human natural antibodies primarily in its IgM pool, it is supported that Pentaglobin can inhibit binding of IgG and IgA autoantibodies present in COVID-19 sera to oxLDL similarly as the oxPL-specific mouse monoclonal IgM antibodies did.

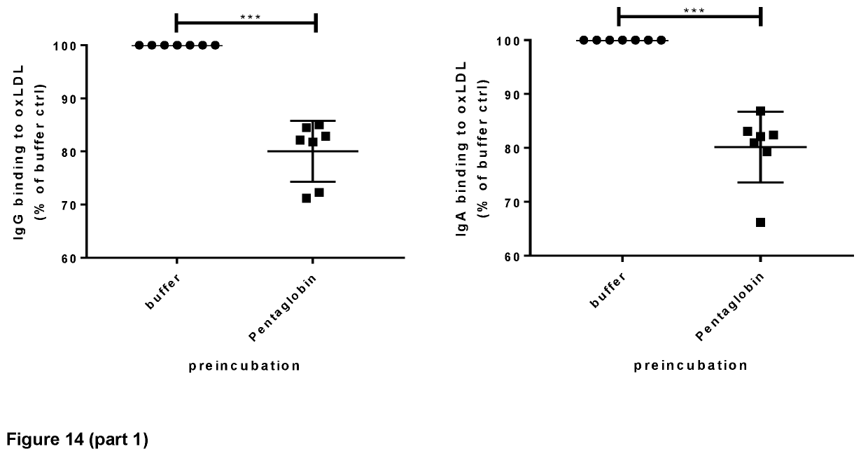

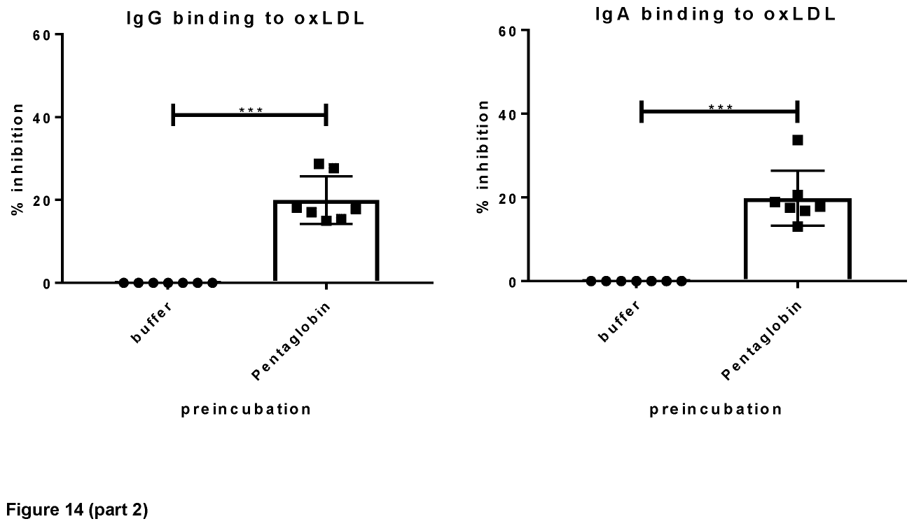

In fact, preincubation of oxLDL-coated wells with Pentaglobin significantly inhibited the binding of autoantibodies from sera of COVID-19 patients to oxLDL by >20%, and this effect was evident for both IgG and IgA autoantibodies (FIG. 14).

These novel findings support the application of Pentaglobin® to treat severely ill COVID-19 patients because of its surprising properties to neutralize different classes of oxPL and OSE and to prevent the formation of immune complexes containing IgG and IgA autoantibodies, thereby showing potential to ameliorate the hyperinflammatory immune responses and to contribute to an rapid improvement of the clinical condition.

### Example 20. Clinical NAD Modulating Intervention Strategy for COVID-19

COVID-19 is a disease with extraordinarily high medical need in terms of both treating and preventing the disease. We anticipate NAD modulation as valuable intervention strategy for both treatment and prevention of COVID-19.

1. Clinical Treatment Trials
a. In a small study with up to 10 patients with severe COVID-19 infection who require mechanistic ventilation or according to medical assessment will require mechanistic ventilation within 1-4 hours due to rapid pulmonary deterioration the natural antibody containing formulation Pentaglobin® is administered at 5 m L/kg at 28 mL/h for 3 consecutive days.
b. Subsequently, a controlled clinical phase II study with up to 50 patients at two centers is commenced. In this trial, effects of potential NAD modulating activity of Pentaglobin® is systematically assessed and correlated with key clinical outcome data.
c. Subsequently, a large phase III multinational multicenter trial is implemented. Moreover, novel natural antibody enriched plasma formulations (i.e. from female donors <25 years of age) are developed for subsequent approval trials, preferably already at a time point with first available interims data from the phase III study.
d. Subsequently, recombinant monoclonal natural antibodies are simultaneously developed for replacement of NAD modulating Pentaglobin® for treating COVID-19 and other NAD associated diseases.

2. Clinical Prevention Trials
Preclinical data indicate potential NAD modulating effects for currently available vaccines such as BCG or Pneumovax.
Following successful preclinical confirmation of this in vitro and in relevant animal models natural antibody inducing vaccination trials with respective compounds are initiated. Since for some of these compounds (e.g. BCG) protection from acquiring severe COVID19 disease has been postulated on the basis of epidemiological data. However, induction of natural antibodies as the potential causative reason for protection has thus far not been shown. Consequently, NAD modulation is systematically analyzed in explorative parts of these trials and also correlated with clinical outcome data.

### Example 21. Clinical Treatment of COVID-19 with Pentaglobin®

5 patients with very severe COVID-19 course of disease and in whom 4 out of 5 required invasive mechanical ventilation were administered with Pentaglobin.

Patient 1:

59 year old male (#1).

Comorbidities: M. Bechterew (morphine-dependent chronical pain patient), osteoporosis, hypertension.

History: On Mar. 28, 2020, presentation at local hospital with abdominal pain and rapid impairment of general condition, positive SARS-CoV-2 swab on the same day. Rapid development of respiratory insufficiency, in the course deterioration and requirement for intubation and invasive CPAP ventilation on April 4. Transfer to ICU UKHD on April 14, at time of admission requirement for catecholamine (noradrenaline). In CT Thorax from April 14, signs of typical COVID-19 pneumonia, suspicious of pre-existing lung fibrosis. On April 16, administration of 10 g Pentaglobin, initiation of Aciclovir therapy after tested positive for HSV-1 in BAL. Cardiopulmonal stabilization, thereafter, change to BIPAP ventilation, tracheostoma. On April 22, spontaneous breathing attempt, not yet well tolerated w/ tachypnea, and pathological breathing mechanics.

Patient 2:

80 year old male (#2).

Comorbidities: M. Bechterew, coronary 3 heart disease, acute myocardial infarction due to RCA occlusion, acute cervical vertebra 7 fracture after collapse.

History: Respiratory infection for 1 week, non-responsive to antibiotics. On April 2, patient was found unconscious at home with head laceration, alarming of emergency physician, at arrival, vigilance significantly reduced yet responsive, SO2 87% at 12 l O2/min, transport to hospital emergency room. Diagnostics: swab Sars-2 positive, CT scan: central lung arterial embolism, typical signs of COVID-19 lung disease. Elevated temperature 38.5° C. In ECG, signs of myocardial infarction (RCA), dilatation and stenting of RCA and recanalization successful, intubation for intervention required, catecholamine dependency. In the course, significant impairment of pulmonary situation, in CT scan on April 12, significant impairment of COVID-19 lung infiltrates with beginning consolidation. Between April 13 and Apr. 15, 2020, administration of 22.5 g Pentaglobin daily. On April 16, temporary impairment of retention parameters yet improved diuresis (hemodialysis initiated), thereafter slow clinical stabilization of kidney and pulmonary situation, on April 20, intermittent CPAP w/slow PEEP reduction over the next days, attempts to intermittently pause assisted ventilation successful, oxygenation not yet satisfactory. On April 22, three subsequent negative tests for Sars-2, thus transfer to cardiologic ICU for further stabilization of the cardiopulmonary situation and planning subsequent surgery of the vertebra fracture.

Patient 3:

62 year old female (#3).

Comorbidities: Adipositas, Klippel-Trenaunay syndrome.

History: On Mar. 29, 2020, presentation at local hospital with fever and dyspnea for 5 days and impairment of general condition, positive SARS-CoV-2 swab on the same day. Rapid development of respiratory insufficiency, in the course deterioration and requirement for intubation and invasive BIPAP ventilation on April 8. Transfer to another local hospital ICU (Schwäbisch Hall) and subsequent transfer to ICU UKHD on April 13, temporarily low dose catecholoamine requirement. In CT Thorax from April 13, signs of typical COVID-19 pneumonia. Detection of free floating thrombus in V. jug., full dose heparinization. On April 14 and 15, administration of 10 g Pentaglobin on each day. Rapid cardiopulmonal improvement thereafter, weaning and extubation without any complications. Initiation of Aciclovir therapy after tested positive for HSV-1 in tracheal fluid on April 19. On April 20, SARS-CoV-2 negative swab. Re-transport to local hospital with 1 l/min O2 via nose.

Patient 4:

76 year old male (#4).

Comorbidities: Diabetes mellitus II insulin dependent, hypertension, dyslipidemia, severe coronary 3 heart disease with bradycardiac atrial fibrillation, NSTEMI w/ high grade LCX stenosis and RCA occlusion.

History: Starting with fever, dyspnea, rapid impairment of general condition since April 3, patient presented at local hospital on April 13. SARS-CoV2 tested positive the day before, exposition by family member. Rapid impairment of cardiopulmonary situation and requirement for intubation and invasive intubation starting April 14. Subsequent development of increased troponin levels and acute renal failure. Transfer to ICU UKHD on April 15. Immediate coronary angiography revealed occluded RCA with sufficient collaterals and proximal LCX Stenosis, PTCA and LCX stenting successfully performed in the same session, catecholamines and hemodialysis required. CT scan on April 19, revealed typical signs of COVID-19 lung disease, suspicion of aortic ulcus of unknown origin. In the course, significant impairment of pulmonary situation, in CT scan on April 12, significant impairment of COVID-19 lung infiltrates with beginning consolidation. On April 16, patient received 10 g Pentaglobin. Development of bradycardiac atrial fibrillation episodes, improvement after clonidine administration stop. Cardiopulmonary stabilization. On April 19, administration of another 10 g Pentaglobin. Last fever episode on April 20, at 38.8° C., reduction of sedation. Since April 21, weaning attempt, since April 22, stabilized cardiopulmonary condition.

Patient 5:

62 year old male (#5).

Comorbidities: None.

History: On Mar. 29, 2020 anamnestic contact to COVID-19 positive person. On April 1, dry cough, fever, chills and impairment of general condition, presentation at local hospital, tested positive in SARS-CoV-2 swab on April 13. Subsequently, continuous increase of O2 demand and development of respiratory insufficiency. On April 15, transport to IMC unit UKHD due to respiratory deterioration. At UKHD, throat swab for SARS-CoV-2 negative. In CT Thorax from the same day signs of typical COVID-19 pneumonia. From April 15 to April 21, highflow oxygenation therapy (HFOT, Optiflow®). On April 16, administration of steroids and 10 g Pentaglobin. In the further course, stabilization of the pulmonary situation allowing for stepwise reduction of apparative oxygenation and termination of HFOT on April 21. Sputum tested for SARS-CoV-2 negative on April 17. Notably, TNT remained stably high without clinical symptoms and alterations in ECG. Re-transport to local hospital in good condition with 1 l/min O2 via nose.

The following Tables show the clinical data determined for Patient 1, Patient 2, Patient 3, Patient 4 and Patient 5, respectively, over the course of time.

Patient #1 (age 59, male)

| | | | Symptoms | x | x | x | x | x | x | x | x | x |
|---|---|---|---|---|---|---|---|---|---|---|---|---|
| | | | Hospitalisation | x | x | x | x | x | x | x | x | x |
| | | | Invasive ventilation | x | x | x | x | x | x | x | x | x |
| Anti-infectives administered | | | Pip/Taz/Caspo | | Pip/Taz/ | Pip/Taz/Caspo | Meropenem/ | Meropenem/ | Meropenem/ | Meropenem/ | Meropenem/ | Meropenem/ |
| | | | Meropenem/Vanco | | Caspo | Meropenem/Vanco | Vanco | Vanco | Vanco | Vanco | Vanco | Vanco |
| | | | Aciclovir with HSV | | | Aciclovir | Aciclovir | Aciclovir | Aciclovir | Aciclovir | Aciclovir | Aciclovir |
| Immunomodulators administered | | | Maraviroc | | Maraviroc | Maraviroc | Maraviroc | Maraviroc | Maraviroc | Maraviroc | Maraviroc | Maraviroc |
| | | | Prednisolon 180 mg | | | Prednisolon | | | | | | |
| | | | Pentaglobin 10 g | | | Pentaglobin | | | | | | |
| | | | SARS-COV-2 result | | negative | negative | | | | negative | negative | |
| Laboratory | | | Days | Day 25 | Day 26 | Day 27 | Day 28 | Day 29 | Day 30 | Day 31 | Day 32 | Day 33 |
| results* | Test | Unit | Normal value | Apr. 14, 2020 | Apr. 15, 2020 | Apr. 16, 2020 | Apr. 17, 2020 | Apr. 18, 2020 | Apr. 19, 2020 | Apr. 20, 2020 | Apr. 21, 2020 | Apr. 22, 2020 |
| Clinical | Sodium | mmol/l | 135-146 | 149 | 149 | 155 | 151 | 155 | 154 | 151 | 148 | 151 |
| chemistry | Potassium | mmol/l | 3.4-4.8 | 4.90 | 4.30 | 4.66 | 4.23 | 4.13 | 4.94 | 4.59 | 4.53 | 4.50 |
| | Creatinine | mg/dl | 10.6-1.2 | 1.36 | 1.79 | 2.20 | 2.27 | 1.81 | 1.40 | 1.24 | 0.99 | 0.92 |
| | GFR using CKD-EPI | | >60 | 56.6 | 40.6 | 31.6 | 30.4 | 40.0 | 54.6 | 63.2 | 83.0 | 90.7 |
| | Urea | mg/dl | <45 | 74 | 95 | 111 | 138 | 153 | 128 | 115 | 90 | 77 |
| | Creatine kinase (CK) | U/l | <190 | 633 | NA | 392 | 190 | 181 | 118 | 164 | 122 | 128 |
| | Troponin T (TNT) | pg/ml | <14 | 56 | 77 | 66 | 53 | 58 | 62 | 61 | 54 | 88 |
| | Lactate dehydrogenase | U/l | <317 | 655 | 516 | 485 | 422 | 428 | 437 | 555 | 482 | 528 |
| | (LDH) | | | | | | | | | | | |
| | GOT/AST | U/l | <46 | 141 | 118 | 123 | 66 | 68 | 93 | 103 | 75 | 60 |
| | GPT/ALT | U/l | <50 | 48 | 44 | 55 | 36 | 38 | 63 | 71 | 59 | 46 |
| | Gamma-glutamyltransferase | U/l | <60 | 85 | 65 | 80 | 64 | 78 | 121 | 109 | 87 | 70 |
| | (GGT) | | | | | | | | | | | |
| | Iron | μmol/l | 14-32 | 7.6 | 1.7 | 2.6 | 6.8 | 1.6 | 1.2 | 2.0 | 2.0 | 2.9 |
| | Triglycerides | mg/dl | <150 | NA | NA | 220 | 183 | 317 | 229 | 189 | 147 | 127 |
| | Albumin | g/l | 30-50 | 30.2 | 28.8 | 28.5 | 30.7 | 32.3 | 28.9 | 30.6 | 27.4 | 26.2 |
| | C-reactive protein (CRP) | mg/l | <5 | 315.7 | 324.5 | 398.9 | 270.9 | 127.7 | 126.2 | 174.9 | 175.9 | 155.6 |
| Hematology | Leucocytes | /nl | 4-10 | 18.14 | 15.94 | 17.80 | 10.80 | 8.49 | 8.64 | 12.29 | 13.47 | 13.55 |
| | Neutrophil granulocytes | % | 50-80 | 1 | 88.1 | 89.0 | 90.6 | 82.0 | 74.9 | 70.6 | 68.1 | NA |
| | (automated) | | | 80.3 | | | | | | | | |
| | Lymphocytes (automated) | % | 25-40 | 14.7 | 7.5 | 6.8 | 4.9 | 9.3 | 12.8 | 15.6 | 17.8 | NA |
| | Eosinophil granulocytes | % | 2-4 | 1.3 | 0.2 | 0.7 | 0.3 | 4.7 | 6.9 | 7.3 | 7.0 | NA |
| | (automated) | | | | | | | | | | | |
| | Lymphocytes (absolute) | /nl | 1.0-4.8 | 2.67 | 1.20 | 1.21 | 0.53 | 0.79 | 1.11 | 1.92 | 2.40 | 1.90 |
| Coagulation | D-Dimer | mg/l | <0.5 | 17.10 | 6.65 | 3.46 | 5.30 | 7.76 | 9.32 | 10.44 | 11.20 | 12.58 |
| | IgG | g/l | 7.0-16.0 | NA | 13.15 | 12.31 | 11.54 | 10.46 | 9.31 | NA | NA | NA |
| | IgA | g/l | 0.7-4.0 | NA | 11.76 | 11.16 | 8.81 | 8.40 | 7.70 | NA | NA | NA |
| | IgN | g/l | 0.4-2.3 | NA | 2.02 | 1.88 | 1.49 | 1.44 | 1.30 | NA | INA | NA |
| | Transferrin | g/l | 2.0-3.6 | 0.62 | 0.82 | 0.60 | 0.55 | 0.90 | 0.86 | 0.90 | 0.91 | 0.88 |
| | Transferrin saturation | % | 16-45 | 49 | 8 | 17 | 49 | 7 | 6 | 9 | 9 | 13 |
| | Ferritin | μg/l | 30-300 | 1794 | 3276 | 2197 | 1766 | 1581 | 1121 | 1460 | 1378 | 1405 |
| | Procalcitonin (PCT), sensitive | ng/ml | <0.05 | 1.16 | 16.75 | 19.75 | 18.51 | 10.38 | 4.51 | 2.07 | 1.10 | 0.68 |
| POCT | pH value (POCT) | | 7.37-7.45 | 7.08 | 7.27 | 7.08 | 7.46 | 7.47 | 7.49 | 7.48 | 7.49 | 7.48 |
| | Carbon dioxide partial pressure | mmHg | 35-45 | NA | 64.6 | 101.3 | 49.6 | 36.2 | 36.3 | 47.1 | 52.9 | 37.3 |
| | (pCO2) (POCT) | | | | | | | | | | | |
| | Oxygen partial pressure (pO2) | mmHg | >arterial 103.4 | 76 | 65 | 57 | 77 | 65 | 61 | 61 | 64 | 59 |
| | (POCT) | | minus (0.42 times | | | | | | | | | |
| | | | age in years) | | | | | | | | | |
| | Base Excess, standard | mmol/L | −2-+3 | −7.5 | −3.2/4.8 | −4.5/6.9 | 5.6 | 3.4 | 4.2 | 4.1 | −6.6/6.5 | 3.5 |
| | Lactate (BGA) | mg/dl | <16 | 15.1 | 17.7 | 19.5 | 18.9 | 18.6 | 12.4 | 13.9 | 12.5 | 8.7 |
| | CSO2 | % | | 91.6 | 91.6 | 86.4 | 94.7 | 92.1 | 89.8 | 91.8 | 91.7 | 91.0 |
| | Blood collection method | | | arterial | arteria | arterial | arterial | arterial | arterial | arteria | arterial | arterial |
| Proteins | sCD25 | U/ml | <900 | NA | NA | 4847 | NA | NA | NA | NA | NA | NA |
| | Soluble transferrin receptor | mg/l | 2.2-5.0 | NA | NA | NA | 2.2 | 2.9 | 3.3 | 3.7 | 4.3 | NA |
| | (sTFR) | | | | | | | | | | | |
| | Ferritin index | | 3.2 | NA | NA | NA | 0.68 | 0.91 | 1.08 | 1.17 | 1.37 | NA |
| | Interleukin 6 | pg/ml | | 350.0 | 157.0 | 270.0 | <2.0 | 37.3 | 89.0 | 75.6 | 50.5 | NA |

*Please note that in case lab testing was performed several times a day, the documented result is the worst result from that day.
NA = not available

Patient #2 (age 80, male)

| | | | Symptoms | x | x | x | x | x | x | x |
|---|---|---|---|---|---|---|---|---|---|---|
| | | | Hospitalisation | x | x | x | x | x | x | x |
| | | | Invasive ventilation | x | x | x | x | x | x | x |
| Anti-infectives administered | | | Pip/Taz/Caspo | Pip/Taz | Pip/Taz | Pip/Taz | Pip/Taz | Pip/Taz | Pip/Taz | Pip/Taz |
| | | | Moxiflox | | | | Caspo | Caspo | Caspo | Caspo |
| | | | Meropenem | | | | | | | |
| | | | Aciclovir with HSV | | | | | | | |
| Immunomodulators administered | | | Maraviroc 2 × 75 mg | | | | | | | |
| | | | Pentaglobin 22.5 g | | | | | | | |
| | | | SARS-COV-2 result | positive | positive | | positive | | | |
| | | | Symptoms | x | x | x | x | x | x | x |
| | | | Hospitalisation | x | x | x | x | x | x | x |
| | | | Invasive ventilation | x | x | x | x | x | x | x |
| Anti-infectives administered | | | Pip/Taz/Caspo | Pip/Taz | Caspo | | Pip/Taz | Pip/Taz | Pip/Taz | Pip/Taz |
| | | | | Caspo | | | | | Caspo | Caspo |
| | | | Meropenem | | | | | | | |
| | | | Aciclovir with HSV | | | | | | | |
| Immunomodulators administered | | | Maraviroc 2 × 75 mg | | | | | | | Maraviroc |
| | | | Pentaglobin 22.5 g | | | | | Pentaglobin | Pentaglobin | Pentaglobin |
| | | | SARS-COV-2 result | positive | | | | positive | | |
| | | | Symptoms | x | x | x | x | x | x | x |
| | | | Hospitalisation | x | x | x | x | x | x | x |
| | | | Invasive ventilation | x | x | x | x | x | x | x |
| Anti-infectives administered | | | Pip/Taz/Caspo | Pip/Taz | Pip/Taz | Pip/Taz | Meropenem | Meropenem | Meropenem | Meropenem |
| | | | Moxiflox | Caspo | Caspo | Caspo | Caspo | Caspo | Caspo | Caspo |
| | | | Meropenem | | | Meropenem | | Aciclovir | Aciclovir | Aciclovir |
| | | | Aciclovir with HSV | | | | | | | |
| Immunomodulators administered | | | Maraviroc 2 × 75 mg | Maraviroc | Maraviroc | Maraviroc | Maraviroc | Maraviroc | Maraviroc | Maraviroc |
| | | | Pentaglobin 22.5 g | | | | | | | |
| | | | SARS-COV-2 result | positive | positive | | | negative | negative | negative |
| Laboratory | | | Days | Day 11 | Day 12 | Day 13 | Day 14 | Day 15 | Day 16 | Day 17 |
| results* | Test | Unit | Normal value | Apr. 2, 2020 | Apr. 3, 2020 | Apr. 4, 2020 | Apr. 5, 2020 | Apr. 6, 2020 | Apr. 7, 2020 | Apr. 8, 2020 |
| Clinical | Sodium | mmol/l | 135-146 | 138 | 142 | 146 | 147 | 150 | 151 | 150 |
| chemistry | Potassium | mmol/l | 3.4-4.8 | 5.03 | 4.62 | 4.82 | 5.87 | 5.15 | 1.78 | 4.57 |
| | Creatinine | mg/dl | 0.6-1.2 | 1.18 | 1.76 | 2.26 | 3.07 | 4.12 | 5.38 | 5.25 |
| | GFR using | | >60 | 57.9 | 35.7 | 26.4 | 18.2 | 12.8 | 9.3 | 9.5 |
| | CKD-EPI | | | | | | | | | |
| | Urea | mg/dl | <45 | 37 | 44 | 55 | 68 | 91 | 135 | 140 |
| | Creatine kinase (CK) | U/l | <190 | 1353 | NA | NA | NA | NA | NA | 79 |
| | Troponin T (TNT) | pg/ml | <14 | 4000 | 6837 | 651 | 637 | 5936 | 5950 | NA |
| | Lactate | U/l | <317 | NA | 658 | 580 | 595 | 434 | 451 | 350 |
| | dehydrogenase | | | | | | | | | |
| | (LDH) | | | | | | | | | |
| | GOT/AST | U/l | <46 | 107 | 254 | 155 | 95 | 57 | 47 | 40 |
| | GPT/ALT | U/l | 50 | 70 | 83 | 62 | 49 | 43 | 37 | 29 |
| | Gamma-glutamyl- | U/l | 60 | 69 | 58 | 50 | 43 | 39 | 54 | 60 |
| | transferase (GGT) | | | | | | | | | |
| | Iron | μmol/l | 14-32 | NA | NA | 1.3 | 0.8 | 1.5 | 7.0 | 4.9 |
| | Triglycerides | mg/dl | <150 | NA | NA | NA | NA | NA | NA | 132 |
| | Albumin | g/l | 30-50 | 37.5 | 35.2 | 34.3 | 31.4 | 31.5 | 28.7 | 28.4 |
| | C-reactive | mg/l | 5 | 130.3 | 151.1 | 273.3 | 291.0 | 304.0 | 238.9 | 155.9 |
| | protein (CRP) | | | | | | | | | |
| Hematology | Leucocytes | /nl | 14-10 | 13.66 | 11.37 | 10.38 | 11.52 | 11.90 | 8.21 | 7.82 |
| | Neutrophil | % | 50-80 | 93.8 | 84.3 | 88.8 | 89.7 | 90.5 | 91.5 | 88.6 |
| | granulocytes | | | | | | | | | |
| | (automated) | | | | | | | | | |
| | Lymphocytes | % | 25-40 | 2.0 | 8.8 | 5.2 | 4.4 | 3.3 | 3.2 | 4.0 |
| | (automated) | | | | | | | | | |
| | Eosinophil | % | 2-4 | 0.1 | 0.2 | 0.8 | 1.0 | 1.0 | 1.8 | 2.3 |
| | granulocytes | | | | | | | | | |
| | (automated) | | | | | | | | | |
| | Lymphocytes | /nl | 1.0-4.8 | 0.27 | 1.00 | 0.54 | 0.51 | 0.39 | 0.25 | 0.31 |
| | (absolute) | | | | | | | | | |
| Coagulation | D-Dimer | mg/l | <0.5 | 32.16 | NA | 3.18 | 2.35 | 2.06 | 2.95 | 3.62 |
| | IgG | g/l | 7.0-16.0 | NA | NA | NA | NA | NA | NA | NA |
| | IgA | g/l | 0.7-4.0 | VA | VA | NA | NA | NA | NA | NA |
| | IgM | g/l | 0.4-2.3 | NA | NA | NA | NA | NA | NA | NA |
| | Transferrin | | 2.0-3.6 | 1.60 | 1.49 | 1.06 | 0.95 | 0.79 | 0.75 | 0.75 |
| | Transferrin saturation | % | 16-45 | NA | NA | 5 | 3 | 8 | 37 | 26 |
| | Ferritin | μg/l | 30-300 | 178 | 207 | 198 | 220 | 214 | 221 | 205 |
| | Procalcitonin (PCT), | ng/ml | <0.05 | 0.18 | 0.42 | 0.68 | 0.50 | 0.63 | 0.98 | 1.08 |
| | sensitive | | | | | | | | | |
| POCT | pH value (POCT | | 7.37-7.45 | 7.08 | 7.20 | 7.18 | 7.19 | 7.22 | 7.23 | 7.25 |
| | Carbon dioxide | mmHg | 35-45 | 73.3 | 55.2 | 58.1 | 60.7 | 55.1 | 57.2 | 53.1 |
| | partial pressure | | | | | | | | | |
| | (pCO2) (POCT) | | | | | | | | | |
| | Oxygen partial | mmHg | >arterial 103.4 | 73 | 82 | 69 | 76 | 65 | 46 | 65 |
| | pressure (pO2) | | minus (0.42 times | | | | | | | |
| | (POCT) | | age in years) | | | | | | | |
| | Base Excess, standard | mmol/L | −2-+3 | −9.8 | −7.9 | −7.2 | −6.5 | −8.0 | −4.9 | −4.6 |
| | Lactate (BGA) | mg/dl | <16 | 13.5 | 15.0 | 13.1 | 12.5 | 13.0 | 13.3 | 10.5 |
| | cSO2 | % | | 91.0 | 94.8 | 91.0 | 93.0 | 89.7 | 75.1 | 91.0 |
| | Blood collection | | | arterial | arterial | arterial | arterial | arterial | arterial | arterial |
| | method | | | | | | | | | |
| Proteins | sCD25 | U/ml | <900 | NA | NA | NA | NA | NA | NA | NA |
| | Soluble transferrin | mg/l | 2.2-5.0 | NA | VA | NA | NA | NA | NA | NA |
| | receptor | | | | | | | | | |
| | (sTFR) | | | | | | | | | |
| | Ferritin index | | 13.2 | NA | NA | NA | NA | NA | NA | NA |
| | Interleukin 6 | pg/ml | | 122.0 | NA | NA | 365.0 | 297.0 | 180.0 | 96.2 |
| Laboratory | | | Days | Day 18 | Day 19 | Day 20 | Day 21 | Day 22 | Day 23 | Day 24 |
| results* | Test | Unit | Normal value | Apr. 9, 2020 | Apr. 10, 2020 | Apr. 11, 2020 | Apr. 12, 2020 | Apr. 13, 2020 | Apr. 14, 2020 | Apr. 15, 2020 |
| Clinical | Sodium | mmol/l | 135-146 | 1.53 | 151 | 152 | 148 | 147 | 146 | 149 |
| chemistry | Potassium | mmol/l | 3.4-4.8 | 4.64 | 4.22 | 4.30 | 4.96 | 6.19 | 4.39 | 4.63 |
| | Creatinine | mg/dl | 0.6-1.2 | 5.14 | 5.02 | 4.95 | 4.86 | 4.94 | 5.09 | 4.87 |
| | GFR using | | >60 | 9.8 | 10.1 | 10.2 | 10.5 | 10.3 | 9.9 | 10.4 |
| | CKD-EPI | | | | | | | | | |
| | Urea | mg/dl | <45 | 143 | 149 | 1.59 | 168 | 169 | 205 | 227 |
| | Creatine kinase (CK) | U/l | <190 | NA | NA | NA | NA | 78 | NA | 1058 |
| | Troponin T (TNT) | pg/ml | <14 | 4020 | 3649 | 2796 | 2582 | NA | 767 | 522 |
| | Lactate | U/l | <317 | 360 | 338 | 325 | 383 | 339 | 377 | 305 |
| | dehydrogenase | | | | | | | | | |
| | (LDH) | | | | | | | | | |
| | GOT/AST | U/l | <46 | 43 | 41 | 46 | 64 | 66 | 167 | 125 |
| | GPT/ALT | U/l | 50 | 28 | 27 | 27 | 29 | 32 | 48 | 50 |
| | Gamma-glutamyl- | U/l | 60 | 75 | 75 | 85 | 106 | 107 | 107 | 101 |
| | transferase (GGT) | | | | | | | | | |
| | Iron | μmol/l | 14-32 | 4.2 | 3.5 | 3.7 | 4.5 | 2.7 | 7.0 | 11.2 |
| | Triglycerides | mg/dl | <150 | NA | NA | 136 | NA | 138 | NA | 123 |
| | Albumin | g/l | 30-50 | 30.6 | 30.9 | 31.2 | 29.9 | 34.5 | 31.5 | 29.6 |
| | C-reactive | mg/l | 5 | 146.6 | 161.6 | 174.7 | 185.9 | 196.6 | 151.9 | NA |
| | protein (CRP) | | | | | | | | | |
| Hematology | Leucocytes | /nl | 14-10 | 8.76 | 8.68 | 10.46 | 9.43 | 12.34 | 12.10 | 12.29 |
| | Neutrophil | % | 50-80 | 85.5 | 85.8 | 83.6 | 79.8 | 88.9 | 90.6 | NA |
| | granulocytes | | | | | | | | | |
| | (automated) | | | | | | | | | |
| | Lymphocytes | % | 25-40 | 4.9 | 4.4 | 6.4 | 9.6 | 5.6 | 4.8 | NA |
| | (automated) | | | | | | | | | |
| | Eosinophil | % | 2-4 | 1.9 | 2.1 | 1.9 | 1.7 | 0.9 | 0.1 | NA |
| | granulocytes | | | | | | | | | |
| | (automated) | | | | | | | | | |
| | Lymphocytes | /nl | 1.0-4.8 | 0.43 | 0.38 | 0.67 | 0.91 | 0.55 | 0.58 | NA |
| | (absolute) | | | | | | | | | |
| Coagulation | D-Dimer | mg/l | <0.5 | 4.31 | 3.00 | 2.41 | 2.92 | 3.1 | 3.75 | NA |
| | IgG | g/l | 7.0-16.0 | NA | NA | NA | NA | NA | NA | NA |
| | IgA | g/l | 0.7-4.0 | NA | NA | NA | NA | NA | NA | NA |
| | IgM | g/l | 0.4-2.3 | NA | NA | NA | NA | NA | NA | NA |
| | Transferrin | | 2.0-3.6 | 0.74 | 0.87 | 1.03 | 1.06 | 1.09 | 1.22 | 1.51 |
| | Transferrin saturation | % | 16-45 | 23 | 16 | 14 | 17 | 10 | 23 | 30 |
| | Ferritin | μg/l | 30-300 | 195 | 174 | 172 | 181 | 209 | 253 | 189 |
| | Procalcitonin (PCT), | ng/ml | <0.05 | 0.83 | 0.76 | 0.65 | 0.56 | 0.55 | 0.46 | 0.38 |
| | sensitive | | | | | | | | | |
| POCT | pH value (POCT | | 7.37-7.45 | 7.20 | 7.29 | 7.45 | 7.22 | 7.23 | 7.32 | 7.30 |
| | Carbon dioxide | mmHg | 35-45 | 68.1 | 55.2 | 47.3 | 68.9 | 66.7 | 51.9 | 49.6 |
| | partial pressure | | | | | | | | | |
| | (pCO2) (POCT) | | | | | | | | | |
| | Oxygen partial | mmHg | >arterial 103.4 | 64 | 64 | 64 | 68 | 76 | 69 | 72 |
| | pressure (pO2) | | minus (0.42 times | | | | | | | |
| | (POCT) | | age in years) | | | | | | | |
| | Base Excess, standard | mmol/L | −2-+3 | 3.2 | 2.8 | 5.0 | 3.5 | 2.4 | −2.2 | −1.0 |
| | Lactate (BGA) | mg/dl | <16 | 11.2 | 12.2 | 12.6 | 11.4 | 15.7 | 15.0 | 11.8 |
| | cSO2 | % | | 90.1 | 90.4 | 91.8 | 88.7 | 93.3 | 92.3 | 93.5 |
| | Blood collection | | | arterial | arterial | arterial | arterial | arterial | arterial | arterial |
| | method | | | | | | | | | |
| Proteins | sCD25 | U/ml | <900 | NA | NA | NA | NA | NA | NA | NA |
| | Soluble transferrin | mg/l | 2.2-5.0 | NA | NA | NA | NA | NA | NA | NA |
| | receptor | | | | | | | | | |
| | (sTFR) | | | | | | | | | |
| | Ferritin index | | 13.2 | NA | NA | NA | NA | NA | NA | NA |
| | Interleukin 6 | pg/ml | | 92.9 | 100.0 | 68.2 | 40.0 | 12.3 | <2 | NA |
| Laboratory | | | Days | Day 25 | Day 26 | Day 27 | Day 28 | Day 29 | Day 30 | Day 31 |
| results* | Test | Unit | Normal value | Apr. 16, 2020 | Apr. 17, 2020 | Apr. 18, 2020 | Apr. 19, 2020 | Apr. 20, 2020 | Apr. 21, 2020 | Apr. 22, 2020 |
| Clinical | Sodium | mmol/l | 135-146 | 151 | 152 | 149 | 151 | 151 | 149 | 148 |
| chemistry | Potassium | mmol/l | 3.4-4.8 | 4.70 | 4.59 | 5.07 | 5.16 | 4.55 | 4.98 | 5.33 |
| | Creatinine | mg/dl | 0.6-1.2 | 4.93 | 3.97 | 2.79 | 3.02 | 2.71 | 2.56 | 2.49 |
| | GFR using | | >60 | 10.3 | 13.4 | 20.5 | 18.6 | 21.2 | 22.7 | 23.5 |
| | CKD-EPI | | | | | | | | | |
| | Urea | mg/dl | <45 | 242 | 194 | 127 | 136 | 142 | 145 | 145 |
| | Creatine kinase (CK) | U/l | <190 | 480 | 294 | 185 | 181 | 205 | 115 | 114 |
| | Troponin T (TNT) | pg/ml | <14 | 560 | 542 | 343 | 244 | 169 | 139 | 135 |
| | Lactate | U/l | <317 | 367 | 368 | 410 | 366 | 433 | 307 | 326 |
| | dehydrogenase | | | | | | | | | |
| | (LDH) | | | | | | | | | |
| | GOT/AST | U/l | <46 | 80 | 67 | 66 | 64 | 82 | 76 | 83 |
| | GPT/ALT | U/l | 50 | 41 | 39 | 41 | 41 | 54 | 54 | 55 |
| | Gamma-glutamyl- | U/l | 60 | 101 | 124 | 123 | 2 | 125 | 121 | 124 |
| | transferase (GGT) | | | | | | | | | |
| | Iron | μmol/l | 14-32 | 7.6 | 4.7 | 4.5 | 5.3 | 11.5 | 5.8 | 5.3 |
| | Triglycerides | mg/dl | <150 | 215 | 150 | 156 | 123 | 116 | 115 | 103 |
| | Albumin | g/l | 30-50 | 28.9 | 30.4 | 30.5 | 31.0 | 33.3 | 31.1 | 31.6 |
| | C-reactive | mg/l | 5 | 18.4 | 120.5 | 160.3 | 134.9 | 101.9 | 75.4 | 79.9 |
| | protein (CRP) | | | | | | | | | |
| Hematology | Leucocytes | /nl | 14-10 | 8.83 | 10.76 | 13.55 | 14.80 | 13.87 | 13.41 | 13.65 |
| | Neutrophil | % | 50-80 | 82.4 | 81.6 | 82.8 | 82.51 | 80.2 | 79.5 | 82.5 |
| | granulocytes | | | | | | | | | |
| | (automated) | | | | | | | | | |
| | Lymphocytes | % | 25-40 | 7.9 | 9.3 | 8.4 | NA | 9.5 | 9.2 | 7.8 |
| | (automated) | | | | | | | | | |
| | Eosinophil | % | 2-4 | .0 | 3.0 | 3.4 | 3.8 | 4.9 | 4.6 | 4.0 |
| | granulocytes | | | | | | | | | |
| | (automated) | | | | | | | | | |
| | Lymphocytes | /nl | 1.0-4.8 | 0.70 | 1.00 | 1.14 | 1.27 | 1.32 | 1.23 | 1.06 |
| | (absolute) | | | | | | | | | |
| Coagulation | D-Dimer | mg/l | <0.5 | 5.35 | 5.31 | 3.79 | 4.81 | 5.94 | 6.92 | 7.05 |
| | IgG | g/l | 7.0-16.0 | NA | NA | NA | 18.16 | NA | NA | NA |
| | IgA | g/l | 0.7-4.0 | NA | NA | NA | 6.62 | NA | NA | NA |
| | IgM | g/l | 0.4-2.3 | NA | NA | NA | 1.74 | NA | NA | NA |
| | Transferrin | | 2.0-3.6 | 1.67 | 1.59 | 1.49 | 1.38 | 1.42 | 1.59 | 1.57 |
| | Transferrin saturation | % | 16-45 | 18 | 12 | 12 | 15 | 32 | 15 | 13 |
| | Ferritin | μg/l | 30-300 | 143 | 134 | 157 | 163 | 201 | 244 | 277 |
| | Procalcitonin (PCT), | ng/ml | <0.05 | 0.29 | 0.35 | 0.25 | 0.24 | 0.23 | 0.26 | 0.29 |
| | sensitive | | | | | | | | | |
| POCT | pH value (POCT | | 7.37-7.45 | 7.33 | 7.36 | 7.39 | 7.36 | 7.30 | 7.46 | 7.38 |
| | Carbon dioxide | mmHg | 35-45 | 52.5 | 35.0 | 31.1 | 31.9 | 30.7 | 27.5 | 30.7 |
| | partial pressure | | | | | | | | | |
| | (pCO2) (POCT) | | | | | | | | | |
| | Oxygen partial | mmHg | >arterial 103.4 | 69 | 42 | 63 | 71 | 62 | 74 | 64 |
| | pressure (pO2) | | minus (0.42 times | | | | | | | |
| | (POCT) | | age in years) | | | | | | | |
| | Base Excess, standard | mmol/L | −2-+3 | −3.5 | −2.3 | −4.6 | −6.7 | −7.0 | −7.4 | −5.1 |
| | Lactate (BGA) | mg/dl | <16 | 12.9 | 14.2 | 14.6 | 13.3 | 14.0 | 15.9 | 10.6 |
| | cSO2 | % | | 92.0 | 71.1 | 91.3 | 92.9 | 89.7 | 94.6 | 92.8 |
| | Blood collection | | | arterial | arterial | arterial | arterial | arterial | arterial | arterial |
| | method | | | | | | | | | |
| Proteins | sCD25 | U/ml | <900 | NA | NA | NA | NA | NA | NA | NA |
| | Soluble transferrin | mg/l | 2.2-5.0 | 4.6 | 4.4 | 5.7 | 5.9 | 5.8 | 6.1 | NA |
| | receptor | | | | | | | | | |
| | (sTFR) | | | | | | | | | |
| | Ferritin index | | 13.2 | 2.13 | 2.07 | 2.60 | 2.67 | 2.52 | 2.56 | NA |
| | Interleukin 6 | pg/ml | | 23.4 | 45.6 | 19.0 | 11.8 | 7.4 | 14.4 | NA |

*Please note that in case lab testing was performed several times a day, the documented result is the worst result from that day.
NA = not available

Patient #3 (age 62, female)

| | | | Symptoms | x | x | x | x | x | x | x | x | x | x |
|---|---|---|---|---|---|---|---|---|---|---|---|---|---|
| | | | Hospitalisation | x | x | x | x | x | x | x | x | x | x |
| | | | Invasive ventilation | x | x | x | x | x | x | | | | |
| Anti-infectives administered | | | Pip/Taz/Caspo | Pip/Taz | Pip/Taz | Pip/Taz/ | Pip/Taz/ | Pip/Taz/ | Pip/Taz/ | Pip/Taz/ | Pip/Taz/ | Pip/Taz/ | Pip/Taz/ |
| | | | Aciclovir with HSV | | | Caspo | Caspo | Caspo | Caspo | Caspo | Caspo | Caspo | Caspo |
| | | | | | | | | | | Aciclovir | Aciclovir | Aciclovir | Aciclovir |
| Immunomodulators administered | | | Maraviroc | | Maraviroc | Maraviroc | Maraviroc | Maraviroc | Maraviroc | Maraviroc | Maraviroc | Maraviroc | Maraviroc |
| | | | Prednisolon 100 mg | | Prednisolon | | | | | | | | |
| | | | Pentaglobin 10 g | | Pentaglobin | Pentaglobin | | | | | | | |
| | | | SARS-COV-2 result | positive | | negative | | | | | negative | | |
| Laboratory | | | Days | Day 21 | Day 22 | Day 23 | Day 24 | Day 25 | Day 26 | Day 27 | Day 28 | Day 29 | Day 30 |
| results* | Test | Unit | Normal value | Apr. 13, 2020 | Apr. 14, 2020 | Apr. 15, 2020 | Apr. 16, 2020 | Apr. 17, 2020 | Apr. 18, 2020 | Apr. 19, 2020 | Apr. 20, 2020 | Apr. 21, 2020 | Apr. 22, 2020 |
| Clinical | Sodium | mmol/l | 135-146 | 150 | 150 | 155 | 154 | 149 | 145 | 140 | 140 | 138 | NA |
| chemistry | Potassium | mmol/l | 3.4-4.8 | 4.55 | 5.55 | 4.63 | 4.68 | 4.46 | 4.81 | 4.72 | 4.45 | 4.49 | NA |
| | Creatinine | mg/dl | 0.6-1.2 | 1.15 | 1.01 | 0.84 | 0.72 | 0.70 | 0.62 | 0.51 | 0.53 | 0.48 | NA |
| | GFR using CKD-EPI | | >60 | 51.1 | 59.8 | 74.7 | 90.0 | 93.2 | 97.0 | 103.4 | 102.1 | 105.5 | NA |
| | Urea | mg/dl | <45 | 85 | 79 | 77 | 58 | 36 | 24 | 17 | 26 | 21 | NA |
| | Creatine kinase (CK) | U/l | <190 | 159 | 133 | 184 | 81 | 45 | 51 | 49 | 40 | 28 | NA |
| | Troponin T (TNT) | pg/ml | <14 | 24 | 20 | 27 | 27 | 23 | 22 | 14 | 20 | 21 | NA |
| | Lactate dehydrogenase | U/l | <317 | 517 | 530 | 349 | 363 | 388 | 382 | 475 | 280 | 349 | NA |
| | (LDH) | | | | | | | | | | | | |
| | GOT/AST | U/l | <46 | 107 | 81 | 59 | 61 | 64 | 53 | 58 | 41 | 50 | NA |
| | GPT/ALT | U/l | <50 | 92 | 83 | 55 | 57 | 57 | 58 | 60 | 52 | 52 | NA |
| | Gamma- | U/l | <60 | 308 | 297 | 215 | 205 | 208 | 191 | 181 | 168 | 167 | NA |
| | glutamyltransferase | | | | | | | | | | | | |
| | (GGT) | | | | | | | | | | | | |
| | Iron | μmol/1 | 14-32 | 4.2 | 4.6 | 7.2 | 5.5 | 4.3 | 5.2 | 7.3 | 9.4 | 9.8 | NA |
| | Triglycerides | mg/dl | <150 | NA | 176 | 193 | 198 | 167 | 190 | 221 | 176 | 200 | NA |
| | Albumin | g/l | 30-50 | 32.5 | 34.0 | 28.0 | 30.2 | 29.9 | 29.2 | 30.6 | 33.0 | 33.8 | NA |
| | C-reactive protein (CRP) | mg/l | <5 | 89.3 | 90.7 | 41.6 | 29.6 | 32.5 | 24.6 | 19.2 | 15.8 | 14.4 | NA |
| Hematology | Leucocytes | /nl | 4-10 | 22.97 | 17.59 | 6.79 | 6.94 | 7.27 | 7.41 | 8.09 | 7.30 | 7.36 | NA |
| | Neutrophil granulocytes | % | 50-80 | 73.7 | 80.6 | 85.2 | 79.0 | 76.2 | 73.2 | 77.5 | 74.9 | 73.0 | NA |
| | (automated) | | | | | | | | | | | | |
| | Lymphocytes (automated) | % | 25-40 | 16.4 | 4.4 | 8.0 | 12.5 | 13.6 | 15.5 | 10.9 | 14.6 | 15.9 | NA |
| | Eosinophil granulocytes | % | 2-4 | 5.2 | 0.3 | 0.3 | 3.2 | 4.0 | 4.8 | 6.1 | 4.1 | 3.8 | NA |
| | (automated) | | | | | | | | | | | | |
| | Lymphocytes (absolute) | /nl | 1.0-4.8 | 2.88 | 0.77 | 0.54 | 0.87 | 0.99 | 1.15 | 0.88 | 1.07 | 1.17 | NA |
| Coagulation | D-Dimer | mg/l | <0.5 | 10.99 | 5.31 | 3.11 | 4.66 | 5.56 | 7.94 | 8.71 | 5.77 | 6.58 | NA |
| | IgG | g/l | 7.0-16.0 | VA | NA | NA | NA | NA | 13.81 | 13.39 | NA | NA | NA |
| | IgA | g/l | 0.7-4.0 | NA | NA | NA | NA | NA | 2.47 | 2.39 | NA | NA | NA |
| | IgM | g/l | 0.4-2.3 | NA | NA | NA | NA | NA | 1.21 | 1.20 | NA | NA | NA |
| | Transferrin | g/l | 2.0-3.6 | 0.90 | 0.97 | 1.06 | 1.10 | 1.00 | 1.10 | 1.08 | 1.49 | 1.49 | NA |
| | Transferrin saturation | % | 16-45 | 19 | 19 | 34 | 20 | 17 | 19 | 27 | 25 | 26 | NA |
| | Ferritin | μg/l | 30-300 | 828 | 1181 | 500 | 357 | 365 | 343 | 360 | 305 | 379 | NA |
| | Procalcitonin (PCT), | ng/ml | <0.05 | 0.10 | 2.62 | 2.70 | 1.07 | 0.43 | 0.26 | 0.15 | 0.13 | 0.09 | NA |
| | sensitive | | | | | | | | | | | | |
| POCT | pH value (POCT) | | 7.37-7.45 | 6.90 | 6.97 | 7.51 | 7.49 | 7.48 | 7.47 | 7.45 | 7.48 | 7.48 | NA |
| | Carbon dioxide partial | mmHg | 35-45 | 172.9 | 158.8 | 51.8 | 48.7 | 44.1 | 31.8 | 33.4 | 35.2 | 35.4 | NA |
| | pressure | | | | | | | | | | | | |
| | (pCO2) (POCT) | | | | | | | | | | | | |
| | Oxygen partial pressure | mmHg | >arterial 103.4 | 77 | 68 | 67 | 75 | 68 | 82 | 77 | 69 | 84 | NA |
| | (pO2) (POCT) | | minus (0.42 times | | | | | | | | | | |
| | | | age in years) | | | | | | | | | | |
| | Base Excess, standard | mmol/L | −2-+3 | −4.0 | 11.4 | 12.1 | 8.1 | 6.4 | −3.3 | −0.7 | −0.8 | 1.2 | NA |
| | Lactate (BGA) | mg/dl | <16 | 16.5 | 10.7 | 11.6 | 7.3 | 7.9 | 6.7 | 7.4 | 7.3 | 7.0 | NA |
| | cSO2 | % | | 89.2 | 93.4 | 94.8 | 95.8 | 94.3 | 96.4 | 95.3 | 94.2 | 96.8 | NA |
| | Blood collection method | | | arterial | arterial | arterial | arterial | arterial | arterial | arterial | arterial | arterial | NA |
| Proteins | sCD25 | U/ml | <900 | NA | NA | NA | NA | NA | NA | NA | NA | NA | NA |
| | Soluble transferrin receptor | mg/l | 2.2-5.0 | NA | NA | 2.9 | 2.9 | 3.2 | 4.3 | 4.5 | 4.4 | 4.5 | NA |
| | (sTFR) | | | | | | | | | | | | |
| | Ferritin index | | 3.2 | NA | NA | 1.11 | 1.14 | 1.25 | 1.70 | 1.76 | 1.77 | 1.75 | NA |
| | Interleukin 6 | pg/ml | | 23.3 | NA | <2.0 | <2.0 | <2.0 | <2.0 | <2.0 | <2.0 | <2.0 | NA |

*Please note that in case lab testing was performed several times a day, the documented result is the worst result from that day.
NA = not available

Patient #4 (age 76, male)

| | | | Symptoms | x | x | x | x | x | x | x | x |
|---|---|---|---|---|---|---|---|---|---|---|---|
| | | | Hospitalisation | x | x | x | x | x | x | x | x |
| | | | Invasive ventilation | x | x | x | x | x | x | x | x |
| Anti-infectives administered | | | Pip/Taz/Caspo | Pip/Taz/ | Pip/Taz/Caspo | Meropenem/ | Meropenem/Vanco | Meropenem/Vanco | Meropenem/Vanco | Meropenem/ | Meropenem/ |
| | | | Meropenem/Vanco | Caspo | Meropenem/ | Vanco | | | | Vanco | Vanco |
| | | | | | Vanco | | | | | | |
| Immunomodulators administered | | | Maraviroc | Maraviroc | Maraviroc | Maraviroc | Maraviroc | Maraviroc | Maraviroc | Maraviroc | Maraviroc |
| | | | Prednisolon 80 mg | | Prednisolon | | | | | | |
| | | | Pentaglobin 10 g | | Pentaglobin | | | Pentaglobin | | | |
| | | | SARS-COV-2 | | | | | | positive | positive | |
| | | | result | | | | | | | | |
| | | | Days | Day 13 | Day 14 | Day 15 | Day 16 | Day 17 | Day 18 | Day 19 | Day 20 |
| Laboratory | Test | Unit | Normal value | Apr. 15, 2020 | Apr. 16, 2020 | Apr. 17, 2020 | Apr. 18, 2020 | Apr. 19, 2020 | Apr. 20, 2020 | Apr. 21, 2020 | Apr. 22, 2020 |
| Clinical | Sodium | mmol/l | 135-146 | 142 | 142 | 141 | 142 | 139 | 139 | 139 | 139 |
| chemistry | Potassium | mmol/l | 3.4-4.8 | 4.60 | 5.07 | 4.90 | 5.61 | 5.19 | 4.82 | 5.08 | 4.43 |
| | Creatinine | mg/dl | 0.6-1.2 | 5.37 | 5.25 | 6.33 | 6.54 | 5.64 | 6.13 | 6.35 | 5.25 |
| | GFR using CKD-EPI | | >60 | 9.5 | 9.8 | 7.8 | 7.5 | 9.0 | 8.1 | 7.8 | 9.8 |
| | Urea | mg/dl | <45 | 148 | 119 | 134 | 133 | 122 | 140 | 126 | 115 |
| | Creatine kinase (CK) | U/l | <190 | 2207 | 1786 | 1182 | 689 | 1172 | 738 | 1013 | 563 |
| | Troponin T (TNT) | pg/ml | <14 | 881 | 993 | 809 | 990 | 830 | 975 | 857 | 658 |
| | Lactate dehydrogenase | U/l | <317 | 935 | 771 | 576 | 605 | 541 | 439 | 405 | 404 |
| | (LDH) | | | | | | | | | | |
| | GOT/AST | U/l | <46 | 93 | 83 | 68 | 57 | 60 | 55 | 82 | 106 |
| | GPT/ALT | U/l | <50 | 37 | 31 | 28 | 26 | 26 | 27 | 32 | 45 |
| | Gamma-glutamyltransferase | U/l | <60 | 26 | 26 | 32 | 26 | 26 | 24 | 30 | 35 |
| | (GGT) | | | | | | | | | | |
| | Iron | μmol/l | 14-32 | 1.4 | 1.7 | 2.5 | 2.7 | 2.7 | 1.8 | 1.8 | 2.6 |
| | Triglycerides | mg/dl | <150 | 167 | 139 | 142 | 177 | 133 | 116 | 97 | 96 |
| | Albumin | g/l | 30-50 | 30.9 | 29.7 | 29.7 | 27.6 | 29.7 | 29.2 | 28.4 | 25.8 |
| | C-reactive protein (CRP) | mg/l | <5 | 335.8 | 395.2 | 349.3 | 182.7 | 208.9 | 217.7 | 198.8 | 148.2 |
| Hematology | Leucocytes | /nl | 4-10 | 15.57 | 10.38 | 7.10 | 6.60 | 2.67 | 5.57 | 5.06 | 3.78 |
| | Neutrophil granulocytes | % | 50-80 | 93.4 | 87.3 | 91.0 | 82.8 | 84.5 | 88.9 | 85.8 | 80.4 |
| | (automated) | | | | | | | | | | |
| | | | | 93.4 | | | | | | | |
| | Lymphocytes (automated) | % | 25-40 | 3.6 | 8.1 | 4.3 | 6.3 | 8.4 | 6.3 | 7.0 | 9.2 |
| | Eosinophil granulocytes | % | 2-4 | 0.3 | 0.6 | 0.1 | 0.8 | 1.9 | 0.8 | 1.2 | 1.8 |
| | (automated) | | | | | | | | | | |
| | Lymphocytes (absolute) | /nl | 1.0-4.8 | 0.56 | 0.84 | 0.31 | 0.42 | 0.22 | 0.35 | 0.35 | 0.35 |
| Coagulation | D-Dimer | mg/l | <0.5 | 1.95 | 1.67 | 1.88 | 2.16 | 4.58 | 2.74 | 4.85 | 9.57 |
| | IgG | g/l | 7.0-16.0 | NA | 5.48 | NA | 6.71 | NA | NA | NA | NA |
| | IgA | g/l | 0.7-4.0 | NA | 2.96 | NA | 3.00 | NA | NA | NA | NA |
| | IgM | g/l | 0.4-2.3 | NA | 0.67 | NA | 0.76 | NA | NA | NA | NA |
| | Transferrin | g/l | 2.0-3.6 | 1.14 | 0.94 | 0.79 | 0.96 | 0.95 | 1.03 | 1.06 | 1.08 |
| | Transferrin saturation | % | 16-45 | 5 | 7 | 13 | 11 | 11 | 7 7 | 7 | 10 |
| | Ferritin | μg/l | 30-300 | 295 | 301 | 245 | 237 | 179 | 203 | 202 | 205 |
| | Procalcitonin (PCT), | ng/ml | <0.05 | 2.60 | 4.32 | 4.83 | 3.34 | 2.53 | 1.85 | 1.45 | 0.99 |
| | sensitive | | | | | | | | | | |
| POCT | pH value (POCT) | | 7.37-7.45 | 7.13 | 7.26 | 7.19 | 7.26 | 7.29 | 7.16 | 7.20 | 7.34 |
| | Carbon dioxide partial | mmHg | 35-45 | 73.8 | 54.5 | 61.3 | 50.6 | 48.9 | 65.2 | 59.6 | 35.8 |
| | pressure | | | | | | | | | | |
| | (pCO2) (POCT) | | | | | | | | | | |
| | Oxygen partial pressure | mmHg | >arterial 103.4 | 80 | 78 | 74 | 73 | 83 | 71 | 60 | 82 |
| | (pO2) (POCT) | | minus (0.42 times | | | | | | | | |
| | | | age in years) | | | | | | | | |
| | Base Excess, standard | mmol/L | −2-+3 | −5.9 | −7.1 | −5.6 | −5.6 | −4.9 | −7.4 | −6.0 | −3.9 |
| | Lactate (BGA) | mg/dl | <16 | 18.8 | 16.2 | 11.8 | 14.4 | 12.5 | 9.2 | 10.9 | 8.5 |
| | CSO2 | % | | 94.5 | 92.9 | 91.4 | 92.7 | 95.2 | 91.2 | 89.6 | 94.4 |
| | Blood collection method | | | arterial | arterial | arterial | arterial | arterial | arterial | arterial | arterial |
| Proteins | sCD25 | U/ml | <900 | 1294 | NA | NA | NA | | | | |
| | Soluble transferrin receptor | mg/l | 2.2-5.0 | NA | 4.7 | 4.4 | 4.6 | 4.9 | 5.0 | 5.1 | |
| | (sTFR) | | | | | | | | | | |
| | Ferritin index | | 3.2 | NA | 1.90 | 1.84 | 1.94 | 2.18 | 2.17 | 2.21 | |
| | Interleukin 6 | pg/ml | | 789.0 | 482.0 | 25.9 | 169.0 | 1603.0 | 362.0 | 121.0 | 246.0 |

*Please note that in case lab testing was performed several times a day, the documented result is the worst result from that day.
NA = not available

Patient #5 (age 62, male)

| | | | Symptoms | x | x | x | x | x | x | x | x |
|---|---|---|---|---|---|---|---|---|---|---|---|
| | | | Hospitalisation | x | x | x | x | x | x | x | x |
| | | | Invasive ventilation | | | | | | | | |
| Anti-infectives administered | | | Azithromycin 4d (amb.) | Meropenem | Meropenem | Meropenem | Meropenem | Meropenem | Meropenem | Meropenem | Caspofungin |
| (not all dates available) | | | Unacid 5d (amb.) | Caspofungin | Caspofungin | Caspofungin | Caspofungin | Caspofungin | Caspofungin | Caspofungin | Meropenem |
| | | | Meropenem | | | | | | | | |
| | | | Caspofungin | | | | | | | | |
| Immunomodulators administered | | | Maraviroc | Maraviroc | Maraviroc | Maraviroc | Maraviroc | Maraviroc | Maraviroc | Maraviroc | Maraviroc |
| | | | Prednisolon 80 mg | | Prednisolon | | | | | | |
| | | | Pentaglobin 10 g | | Pentaglobin | | | | | | |
| | | | SARS-COV-2 result | negative | negative | negative | | | negative | | |
| | | | Days | Day 15 | Day 16 | Day 17 | Day 18 | Day 19 | Day 20 | Day 21 | Day 22 |
| Laboratory results* | Test | Unit | Normal value | Apr. 15, 2020 | Apr. 16, 2020 | Apr. 17, 2020 | Apr. 18, 2020 | Apr. 19, 2020 | Apr. 20, 2020 | Apr. 21, 2020 | Apr. 22, 2020 |
| Clinical chemistry | Sodium | mmol/l | 135-146 | 129 | 130 | 134 | 133 | 130 | 130 | 131 | 132 |
| | Potassium | mmol/l | 3.4-4.8 | 4.45 | 4.57 | 3.17 | 3.53 | 2.37 | 4.50 | 3.53 | 3.56 |
| | Creatinine | mg/dl | 0.6-1.2 | 0.52 | 0.49 | 0.49 | 0.53 | 0.48 | 0.58 | 0.55 | 0.51 |
| | GFR using CKD-EPI | | >60 | 114.3 | 117.1 | 117.1 | 113.4 | 118.1 | 109.3 | 111.7 | 115.2 |
| | Urea | mg/dl | <45 | 18 | 20 | 32 | 23 | 19 | 23 | 20 | 20 |
| | Creatine kinase (CK) | U/l | <190 | 298 | 248 | 128 | 157 | 127 | 100 | 92 | 67 |
| | Troponin T (TNT) | pg/ml | <14 | 54 | 53 | 39 | 50 | 50 | 56 | 55 | 53 |
| | Lactate dehydrogenase (LDH) | U/l | <317 | 598 | 414 | 366 | 384 | 360 | 350 | 318 | 360 |
| | GOT/AST | U/l | <46 | 150 | 91 | 82 | 77 | 51 | 40 | 41 | 37 |
| | GPT/ALT | U/l | <50 | 208 | 167 | 143 | 131 | 98 | 89 | 80 | 72 |
| | Gamma-glutamyltransferase | U/l | <60 | 139 | 121 | 105 | 114 | 94 | | 87 | 82 |
| | (GGT) | | | | | | | | | | |
| | Iron | μmol/l | 14-32 | 2.9 | 3.9 | 11.6 | 5.8 | 4.1 | 4.2 | 4.3 | 4.8 |
| | Triglycerides | mg/dl | <150 | 92 | 100 | 112 | 125 | 118 | 140 | 148 | 162 |
| | Albumin | g/l | 30-50 | 31.8 | 30.7 | 27.9 | 28.3 | 28.4 | 32.4 | 32.4 | 31.0 |
| | C-reactive protein (CRP) | mg/l | <5 | 198.5 | 184.4 | 103.4 | 47.6 | 32.9 | 37.5 | 28.5 | 19.2 |
| Hematology | Leucocytes | /nl | 4-10 | 8.03 | 6.38 | 6.29 | 5.66 | 6.02 | 6.96 | 6.17 | 5.12 |
| | Neutrophil granulocytes | % | 50-80 | 84.7 | 88.8 | 79.9 | 81.2 | 82.6 | 79.1 | 80.2 | 74.6 |
| | (automated) | | | | | | | | | | |
| | Lymphocytes (automated) | % | 25-40 | 5.9 | 6.3 | 9.5 | 9.3 | 9.3 | 11.0 | 9.7 | 13.5 |
| | Eosinophil granulocytes | % | 2-4 | 0.6 | 0.2 | 0.2 | 0.5 | 0.9 | 1.1 | 1.3 | 1.6 |
| | (automated) | | | | | | | | | | |
| | Lymphocytes (absolute) | /nl | 1.0-4.8 | 0.47 | 0.40 | 0.60 | 0.53 | 0.58 | 0.77 | 0.60 | 0.69 |
| Coagulation | D-Dimer | mg/l | <0.5 | 5.08 | 4.88 | 4.25 | 3.92 | 3.25 | 2.38 | 2.09 | 1.68 |
| | IgG | g/l | 7.0-16.0 | NA | NA | NA | NA | NA | NA | NA | NA |
| | IgA | g/l | 0.7-4.0 | NA | NA | NA | NA | NA | NA | NA | NA |
| | IgM | g/l | 0.4-2.3 | NA | NA | NA | NA | NA | NA | NA | NA |
| | Transferrin | g/l | 2.0-3.6 | 0.89 | 1.02 | 0.92 | 1.06 | 1.08 | 1.22 | 1.30 | 1.33 |
| | Transferrin saturation | % | 16-45 | 13 | 15 | 50 | 22 | 15 | 14 | 13 | 14 |
| | Ferritin | μg/l | 30-300 | 2261 | 1976 | 2176 | 1597 | 1375 | 1295 | 1486 | 1480 |
| | Procalcitonin (PCT), sensitive | ng/ml | <0.05 | 0.25 | 0.24 | 0.19 | 0.12 | 0.08 | 0.07 | 0.06 | 0.05 |
| POCT | pH value (POCT) | | 7.37-7.45 | 7.47 | 7.48 | 7.52 | 7.55 | 7.52 | 7.55 | 7.56 | 7.49 |
| | Carbon dioxide partial pressure | mmHg | 35-45 | 30.4 | 30.8 | 29.5 | 24.6 | 18.9 | 24.8 | 25.4 | 28.2 |
| | (pCO2) (POCT) | | | | | | | | | | |
| | Oxygen partial pressure (pO2) | mmHg | >arterial 103.4 minus | 38 | 74 | 61 | 60 | 69 | 63 | 66 | 62 |
| | (POCT) | | (0.42 times age in years) | | | | | | | | |
| | Base Excess, standard | mmol/L | −2-+3 | −1.1 | −4.0 | 2.4 | 2.9 | −7.1 | −2.9 | −0.7 | −3.2 |
| | Lactate (BGA) | mg/dl | <16 | 13.9 | 16.6 | 16.3 | 13.9 | 10.4 | 14.8 | 14.7 | 15.5 |
| | CSO2 | | | 73.0 | 93.9 | 90.9 | 91.7 | 93.3 | 91.4 | 93.5 | 91.9 |
| | Blood collection method | | | arterial | arterial | arterial | arterial | arterial | arterial | arterial | arterial |
| Proteins | sCD25 | U/ml | <900 | 824 | NA | INA | NA | NA | NA | NA | NA |
| | Soluble transferrin receptor | mg/l | 2.2-5.0 | NA | 2.4 | 1.9 | 2.5 | 2.7 | 3 .2 | 3.5 | 3.5 |
| | (sTFR) | | | | | | | | | | |
| | Ferritin index | | 3.2 | NA | 0.73 | 0.57 | 0.78 | 0.85 | 1.03 | 1.10 | 1.10 |
| | Interleukin 6 | pg/ml | | 118.0 | 12.5 | 4.4 | 20.9 | 21.5 | 12.4 | 5.6 | 3.9 |

*Please note that in case lab testing was performed several times a day, the documented result is the worst result from that day.
NA = not available

FIG. 1 summarizes the above data of the five patients with deteriorating COVID-19 pneumonia which were treated with Pentaglobin® (P). FIG. 1 shows the determined concentration of the clinical parameters IL-6 (see FIG. 1A), CRP (see FIG. 1B), PCT (see FIG. 1C) and mean daily blood pCO₂ (see FIG. 1D), respectively. CRP, PCT and IL-6 are major inflammatory markers. “P”: administration of Pentaglobin®. Moreover, FIG. 1 indicates the monitored presence (+) or absence (−) of SARS-CoV-2 in bronchoalveolar lavages (BAL).

In summary, it has been shown that 5 patients with very severe COVID-19 course of disease and in whom 4 out of 5 required invasive mechanical ventilation that administration of Pentaglobin improved the clinical conditions in almost all cases.

Notably, because Pentaglobin was not given in the context of a controlled clinical trial administered doses varied from 10 g on one single day up to 22 g daily for 3 consecutive days. In total only one patient has received the approved dose for treatment of bacterial sepsis of 5 mL/kg for 3 consecutive days. Nevertheless, in most patients Interleukin-6 (IL-6) and other inflammatory markers dropped in a highly significant manner along with significant improvement of the clinical condition and most notably ventilation parameters.

Notably, all patients have received the CCR 5 inhibitor Maraviroc as additional experimental therapeutic. Although improvement of inflammatory parameters may at least in part be also attributed to Maraviroc there is clear evidence for a Maraviroc-independent temporal relationship between Pentaglobin administration, reduction of inflammation (11-6), and clinical improvement. Since also pro-inflammatory macrophage mediated effects of Maraviroc have been shown previously it is also possible that Maraviroc at least in some cases may even antagonize anti-inflammatory Pentaglobin effects.

The data of this pre-study collectively indicate significant improvement of severe COVID-19 disease and thereby confirm the concept of successful treatment of NAD disease. A controlled clinical study confirming the therapeutic principle mediated by Pentaglobin is warranted.

### Example 22. Detection of Anti-Nuclear Autoimmunantibodies in COVID-19 Patient Sera

HEp2 Slides (Kallestad), which are commonly used to detect anti-nuclear autoimmune antibodies (ANA) in serum, were incubated with sera derived from three patients with severe COVID-19 (COV #6, COV #7, COV #8) treated in the intensive care unit or with sera from four healthy donors (HD #1, HD #2, HD #3, HD #4). The results are shown in FIG. 3.

As shown in FIG. 3, the top row presents sera diluted 1:5, the bottom row presents results from sera diluted 1:10.

A solution containing ANA included in the test kit was used as positive control. The total IgG concentration of each undiluted serum is indicated below.

The data show significantly stronger staining signal with sera from COVID-19 patients, indicating the presence of ANA in these sera. In contrast, healthy donor control sera showed little to no staining, indicating the absence of ANA.

These data support that the lack of natural antibodies (nABs) can result in the development of autoimmune antibodies during severe COVID-19 courses. The presence of these autoimmune antibodies provides evidence for recurring or long-lasting COVID-19 disease symptoms, supporting that sufficient levels of natural antibodies, provision of monoclonal natural IgMs or IgAs, or preparations enriched for natural antibodies (e.g. Pentaglobin®) in terms of the present invention can prevent the generation or reduce the levels of autoimmune antibodies.

### Summary of Examples 1 to 22

The above data presented herein further support the model underlying the present invention and can be explained by the unusual feature of lung pathogens such as SARS-CoV-2, SARS, or H5N1 influenza virus, to trigger excessive formation of oxPL and OSE according to the mechanisms explained herein.

Viral replication and, consequently, accumulation of oxPL and OSE in lungs of infected patients trigger immune responses involving recruitment and local activation of monocytes, T cells and B cells, including those that produce protective virus-specific IgG and oxPL-specific IgM antibodies. Some of the B-cell clones that produce IgM antibodies toward oxPL and OSE, may become erroneously stimulated to undergo isotype class-switching from IgM to IgA or IgG, e.g., by presentation of antigenic oxidation-derived peptides or viral peptides to T cells. Such class-switched B cells then produce IgA or IgG autoantibodies that bind to oxPL and OSE displayed by many different oxidatively modified structures including apoptotic cells and oxLDL. Indeed, significantly elevated levels of IgG and IgA autoantibodies in the sera of severe COVID-19 patients that potently bound to oxLDL were found, and these autoantibodies likely form oxLDL-IgG- and oxLDL-IgA-immune complexes. OxPL-specific natural IgM antibodies protect from proinflammatory IgG and IgA autoantibodies in different ways, e.g. by blocking oxPL-binding to scavenger receptors, preventing the formation of pathogenic IgG- and IgA-immune complexes, and by facilitating the safe clearance of oxPL-exposing structures. However, in conditions when the balance between the formation and the neutralization of oxPL are disrupted, e.g., when individuals with low levels of endogenous natural IgM and possibly IgA1 antibodies become infected with SARS-CoV-2, the production of proinflammatory isotypes such as IgG and IgA2 autoantibodies get out of control and newly formed IgG- and IgA2-containing immune complexes of oxLDL eventually deposit at distinct sites in the body, e.g. in vascular walls, joints or glomerular capillary walls, where they potently trigger inflammatory responses through scavenger receptor-, Fc-gamma- and Fc-alpha-receptor-mediated activation of dendritic cells, macrophages and neutrophils.

Such autoimmune immune responses become the main driver of the systemic hyperinflammation state observed in the late phase of severe COVID-19 when no virus can be detected anymore, and in the long-term eventually culminate in Lupus-like autoimmune manifestations such as arthritis, vascular damage, acute kidney injury, induction of a procoagulant state and multiorgan damage, and possibly contribute to a phenomena known as Long-COVID. Therefore, individuals exhibiting reduced levels of oxPL- and OSE-specific natural IgM and possibly IgA1 antibodies, which otherwise would neutralize the proinflammatory functions of oxPL-exposing structures, are particularly prone to develop multiorgan hyperinflammation phenomena induced by immune complexes oxPL-IgG or oxPL-IgA2.

This concept further supports that treating COVID-19 patients with severe disease, or Long-COVID patients experiencing ongoing proinflammatory autoimmune conditions, with IgM antibodies, IgG2 or IgG4 antibodies, IgG antibodies carrying modifications to erase Fc-effector functions, or antigen-binding fragments thereof, recognizing oxPL and OSE, leads to significant reduction of the hyperinflammatory state by neutralizing oxPL- and OSE-exposing structures, thereby preventing the formation of pathogenic oxPL-IgG and oxPL-IgA containing immune complexes and facilitating their safe clearance.

### Example 23. Two Monoclonal Antibodies that Bind to Different Danger-Associated Molecular Pattern (DAMPs), Including OSE and DNA

Two monoclonal antibodies that bind to different danger-associated molecular pattern (DAMPs), including OSE and DNA are characterized herein.

These antibodies are structurally described above with reference to SEQ ID NOs: 1 to 6 (corresponding to “Clone 1”) and SEQ ID NOs: 9 to 14 (corresponding to “Clone 2”), respectively (as well as with reference to SEQ ID NOs: 7 to 8 (corresponding to “Clone 1”) and SEQ ID NOs: 15 to 16 (corresponding to “Clone 2”), respectively).

The antibodies are of the IgM isotype and were isolated from single cell-sorted human B cells exhibiting the phenotype of CD5^{pos}CD20^{pos}CD27^{pos}CD43^{pos}CD70^{neg}.

Human B cells exhibiting this phenotype were described to constitute the human counterpart of mouse B1 cells (Griffin, Holodick et al., 2011, J Exp Med, Vol. 208 (1)). Therefore, the two monoclonal antibodies disclosed herein possess characteristics of natural antibodies.

To test for the specificities of the monoclonal antibodies isolated by the method described herein, these antibodies were expressed as fully human IgM molecules and their antigen binding properties were tested. Two clones were identified that bound to at least two antigens that are well described targets for mouse and human natural antibodies. The following table summarizes the results of the binding assay for the two clones disclosed herein.

| | | | MDA- | MDA- | PC- | | |
|---|---|---|---|---|---|---|---|
| | OxLDL | LDL | LDL | BSA | BSA | BSA | DNA |
| Clone 1 | + | − | + | + | + | − | + |
| Clone 2 | + | − | − | − | − | − | + |

Each clone of mouse or human monoclonal antibodies with characteristics of natural antibodies known in the art showed fine specificity for a clearly definable epitope.

For instance, some of the OSE-specific monoclonal IgM antibodies isolated from apoE-deficient mice (including clone E06 used in some of the experiments presented here) showed unique binding specificities for the phosphorylcholine (PC) headgroup exposed by oxLDL, oxPL such as 2-(5-oxovaleryl) phosphatidylcholine (POVPC), PC-protein adducts, or PC-containing polysaccharides, but not to MDA, while other clones specifically bound MDA-modified LDL and MDA-protein adducts, but not to PC epitopes (Shaw et al., 2000, J Clin Invest, Vol. 105(12)).

The mouse monoclonal IgM antibody 509 used in some experiments presented here above showed specificity toward oxidized phosphatidylethanolamine (PE), but not to oxidized phosphatidylcholine, oxidized phosphatidylserine, oxidized phosphatidic acid, or their native non-oxidized counterparts (Bochkov et al., 2016, Biomark Med., Vol. 10 (8)).

Monoclonal antibody LA25 showed exclusive specificity toward the OSE Malondialdehyde-acetaldehyde (MAA), but not to the structurally related OSE MDA (WO/2018/049083, PCT/US2017/050566).

It is, therefore, surprising that the monoclonal antibodies of “Clone 1” and “Clone 2” characterized above bind to at least two epitopes of DAMPs including oxidized LDL, MDA-proteins adducts, PC-protein adducts, and DNA.

Each of this epitope has been described to be implicated in chronic and acute proinflammatory diseases and to be targets for natural antibodies.

Because of this unique feature, the monoclonal antibodies described herein are particularly suitable to be used to treat patients suffering from inflammatory conditions associated with a natural antibody deficiency, as for instance acute pathogen-induced inflammation, acute lung injury, atherosclerosis, and many other conditions. In such patients, oxidative stress and innate immune responses generate multiple forms of DAMPs including oxPL, degradation products such as MDA, and DNA derived from apoptotic cells or neutrophil extracellular traps (NETs), all of which possess strong proinflammatory effects when not cleared efficiently from circulation, e.g. by natural antibodies. It is therefore desirable that monoclonal antibodies used to treat such patients recognize and neutralize as many DAMPs and OSE as possible to achieve anti-inflammatory and beneficial effects, and we demonstrated herein that only the combination of both monoclonal antibodies E06 and 509 showed significant inhibition of binding of autoreactive IgG antibodies in sera from COVID-19 patients to oxLDL. The unique characteristics of the monoclonal antibodies disclosed herein, therefore, provide important advantages over monospecific natural antibodies know in the art.

1. A method of treating or preventing a disorder or disease associated with/related to/caused by a natural IgM/IgA antibody deficiency (NAD) in a subject, comprising administration of a composition comprising an effective amount of at least one human or humanized natural IgM and/or IgA antibody recognizing oxidized phospholipids and/or oxidation-specific epitopes, wherein said disorder or disease is selected from the group consisting of a natural antibody deficient infectious disease, inflammatory disease, neurodegenerative disease, metabolic disease, autoimmune disease, and cardiovascular disease, wherein each said human natural IgM and/or IgA antibody is from a subgroup of the total IgM and/or IgA repertoire of at least one source, said subgroup essentially consisting of antibodies recognizing oxidized phospholipids and/or oxidation-specific epitopes, and wherein said composition contains more than about 35% said human or humanized natural IgM and/or IgA antibody recognizing oxidized phospholipids and/or oxidation-specific epitopes.

2. (canceled)

3. (canceled)

4. The method of claim 1, wherein said source is at least one healthy individual and said subgroup of IgM and/or IgA antibodies is derived from IgM and/or IgA enriched plasma pools from said at least one healthy individuals, said subgroup essentially consisting of antibodies recognizing oxidized phospholipids and/or oxidation-specific epitopes.

5. (canceled)

6. The method of claim 1, wherein said disorder or a disease associated with/related to/caused by natural IgM/IgA antibody deficiency (NAD) is the virus infection disease COVID-19 caused by the β-Coronavirus SARS-CoV2 or is long COVID-19.

7. The method of claim 1, wherein said human or humanized natural IgM and/or IgA antibody is capable of inhibiting the cell-to-cell spread of a virus from an infected cell to an adjacent non-infected cell.

8. The method of claim 1, wherein said human or humanized natural IgM and/or IgA antibody has an anti-inflammatory activity.

9. The method of claim 1, wherein said disorder or disease associated with natural IgM/IgA antibody deficiency (NAD) is an inflammatory disease or a virus infection disease.

10. The method of claim 9, wherein said inflammatory disease is selected from at least one of the group consisting of infectious diseases mediated by respiratory viruses, infectious diseases caused by bacterial infections mediated by gram positive or gram negative pathogens, infectious diseases caused by fungi, infectious diseases caused by parasites, sterile inflammatory diseases, metabolic disorders, neurodegenerative diseases, and autoimmune diseases.

11. The method of claim 9, wherein said virus infection disease is selected from the group consisting of infections by coronaviruses, influenza viruses, parainfluenza viruses, respiratory syncytial viruses (RSV), rhinoviruses, adenoviruses, enteroviruses, human metapneumoviruses, and herpesviruses.

12. The method of claim 1, wherein said composition is a pharmaceutical composition comprising said effective amount of said human or humanized natural IgM and/or IgA antibody recognizing oxidized phospholipids and/or oxidation-specific epitopes and at least one pharmaceutically acceptable excipient.

13. (canceled)

14. (canceled)

15. The method of claim 1, wherein said human or humanized natural IgM and/or IgA antibody recognizes and binds to at least one of phosphorylcholine exposed by oxidized phosphatidylcholine and/or oxidized 1-palmitoyl-2-arachidonoyl-phosphatidylcholine, to oxidized cardiolipin, to oxidized phosphatidylserine, malondialdehyde-modified proteins, 4-hydroxynonenal-modified proteins, 2-(ω-carboxyethyl)-pyrrole-modified proteins, and oligomeric amyloid-β peptide.

16. The method of claim 1, wherein said composition comprises at least one human or humanized natural IgM and/or IgA antibody recognizing oxidized phospholipids and/or oxidation-specific epitopes wherein said antibody comprises the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6, wherein said antibody recognizes and binds to at least one of phosphorylcholine exposed by oxidized phosphatidylcholine and/or oxidized 1-palmitoyl-2-arachidonoyl-phosphatidylcholine, to oxidized cardiolipin, to oxidized phosphatidylserine, malondialdehyde-modified proteins, 4-hydroxynonenal-modified proteins, 2-(ω-carboxyethyl)-pyrrole-modified proteins, and oligomeric amyloid-β peptide; or
wherein said antibody comprises the complementarity determining regions V_{H}CDR1 comprising SEQ ID NO: 9, V_{H}CDR2 comprising SEQ ID NO: 10, V_{H}CDR3 comprising SEQ ID NO: 11, V_{L}CDR1 comprising SEQ ID NO: 12, V_{L}CDR2 comprising SEQ ID NO: 13, and V_{L}CDR3 comprising SEQ ID NO:14, wherein said antibody recognizes and binds to at least one of phosphorylcholine exposed by oxidized phosphatidylcholine and/or oxidized 1-palmitoyl-2-arachidonoyl-phosphatidylcholine, oxidized cardiolipin, oxidized phosphatidylserine, malondialdehyde-modified proteins, 4-hydroxynonenal-modified proteins, 2-(ω-carboxyethyl)-pyrrole-modified proteins, and oligomeric amyloid-β peptide.

17-28. (canceled)

29. The method of claim 1, wherein said at least one human or humanized natural IgM and/or IgA antibody recognizing oxidized phospholipids and/or oxidation-specific epitopes
wherein said is administered in combination with at least one of:
(a) at least one of an inhibitor/antagonist of the Angiotensin-Converting-Enzyme (ACE), an inhibitor/antagonist of the Angiotensin-II-type 1 receptor (AT1R), a compound that modulates the expression of the ACE2 receptor, Ang(1-7), AT2R agonists, and MAS-receptor agonists,
(b) at least one of a compound inhibiting/antagonizing/neutralizing ligands of Receptor of Advanced Glycation Endproducts (RAGE), and an inhibitor/antagonist of RAGE, and
(c) at least one of Granulocyte-Macrophage Colony-Stimulating Factor (GM-CSF) and/of a compound that increases the phagocytic activity of alveolar macrophages (AM), optionally azithromycin.

30. The method of claim 1, wherein said at least one human or humanized natural IgM and/or IgA antibody recognizing oxidized phospholipids and/or oxidation-specific epitopes
wherein said is administered in combination with
an antiviral compound.

31. The method of claim 1, wherein said at least one human or humanized natural IgM and/or IgA antibody recognizing oxidized phospholipids and/or oxidation-specific epitopes
wherein said is administered in combination with an immunomodulator.

32-39. (canceled)

40. The method of claim 8, wherein said anti-inflammatory activity is selected from at least one of reducing the accumulation of free oxidized phospholipids, reducing the accumulation of free oxidized phospholipids in infected lungs, clearing cellular debris in lung tissue, stimulating IL-10 and/or TGFβ secretion, and neutralizing of pro-inflammatory immune responses triggered by cytokines.

41. The method of 10, wherein said infectious diseases mediated by respiratory viruses comprise at least one disease selected from COVID19, long COVID-19, influenza, MERS-COV and SARS-COV, said sterile inflammatory diseases comprise at least one disease selected from cardiovascular diseases, atherosclerosis, coronary heart disease, heart attack, and stroke, said metabolic disorders comprise diabetes mellitus, said neurodegenerative diseases comprise Alzheimer's Disease, and said autoimmune diseases comprise at least one disease selected from Systemic Lupus Erythematodes and Multiple Sclerosis.

42. The method of claim 11, wherein said coronaviruses are selected from at least one of SARS-CoV, SARS-CoV-2, and MERS, and said herpesviruses are selected from at least one of HSV-1, HSV-2, VZV, EBV, HCMV, HHV-6, HHV-7, and HHV-8.

43. The method of claim 30, wherein said antiviral compound is selected from at least one of remdesivir, favipiravir, camostat mesylate, nafamostat mesylate, umifenovir, and stronger neo-minophagen C.

44. The method of claim 31, wherein said immunomodulator is selected from at least one of anti-PD-1, anti-PD-L1, anti-CD40 (agonist), CD40-Ligand, anti-GM-CSF, anti-CSF-1R, anti-CTLA-4, an antibody that binds to at least one cytokine, an antibody that binds to IL-6, an antibody that binds to an IL-6-specific receptor, anti-IL-6R, an antibody that binds to at least one chemokine, anti-CCL2, anti-CCL5, an antibody that binds to a specific chemokine receptor, anti-CCR2, antiCCR5, synthetic molecules that bind to chemokine receptors, Maraviroc CCR5 receptor inhibitor, and dexamethason.


SEQUENCE LISTING (19 sequences)

SEQ ID NO: 1 - protein, 8 aa, Artificial Sequence
  2cVH CDR1 of human monoclonal OSE-specific lgM clone#1
GYTFTSYD

SEQ ID NO: 2 - protein, 8 aa, Artificial Sequence
  2cVH CDR2 of human monoclonal OSE-specific lgM clone#1
MNPNSGNT

SEQ ID NO: 3 - protein, 19 aa, Artificial Sequence
  2cVH CDR3 of human monoclonal OSE-specific lgM clone#1
ARSPSSSWYLRRGNYGMDV

SEQ ID NO: 4 - protein, 9 aa, Artificial Sequence
  2cVL CDR1 of human monoclonal OSE-specific lgM clone#1
SSDVGSYNL

SEQ ID NO: 5 - protein, 3 aa, Artificial Sequence
  2cVL CDR2 of human monoclonal OSE-specific lgM clone#1
EVS

SEQ ID NO: 6 - protein, 10 aa, Artificial Sequence
  2cVL CDR 3 of human monoclonal OSE-specific lgM clone#1
CSYAGSSTFV

SEQ ID NO: 7 - protein, 126 aa, Artificial Sequence
  VH of the human monoclonal OSE-specific lgM clone#1
EVQLVESGAEVKKPGASVKVSCKASGYTFTSYDINWVRQATGQGLEWMGWMNPNSGNTGYAQKFQGRVTMTRNTSISTAYMELSSLRSEDTAVYYCARSPSSSWYLRRGNYGMDVWGQGTTVTVSS

SEQ ID NO: 8 - protein, 110 aa, Artificial Sequence
  VL of the human monoclonal OSE-specific lgM clone#1
NFMLTQPASVSGSPGQSITISCTGTSSDVGSYNLVSWYQQHPGKAPKLMIYEVSKRPSGVSNRFSGSKSGNTASLTISGLQAEDEADYYCCSYAGSSTFVFGTGTKVTVL

SEQ ID NO: 9 - protein, 8 aa, Artificial Sequence
  2cVH CDR1 of human monoclonal OSE-specific lgM clone#2
GFTFSSYA

SEQ ID NO: 10 - protein, 8 aa, Artificial Sequence
  2cVH CDR2 of human monoclonal OSE-specific lgM clone#2
ISGSGGST

SEQ ID NO: 11 - protein, 19 aa, Artificial Sequence
  2cVH CDR3 of human monoclonal OSE-specific lgM clone#2
AKALAKQWLVQGVRGAFDI

SEQ ID NO: 12 - protein, 7 aa, Artificial Sequence
  2cVL CDR1 of human monoclonal OSE-specific lgM clone#2
QSVSSSY

SEQ ID NO: 13 - protein, 3 aa, Artificial Sequence
  2cVL CDR2 of human monoclonal OSE-specific lgM clone#2
GAS

SEQ ID NO: 14 - protein, 9 aa, Artificial Sequence
  2cVL CDR3 of human monoclonal OSE-specific lgM clone#2
QQYGSSRGT

SEQ ID NO: 15 - protein, 126 aa, Artificial Sequence
  VH of the human monoclonal OSE-specific lgM clone#2
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKALAKQWLVQGVRGAFDIWGQGTMVTVSS

SEQ ID NO: 16 - protein, 108 aa, Artificial Sequence
  VL of the human monoclonal OSE-specific lgM clone#2
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRGTFGPGTKVDIK

SEQ ID NO: 17 - DNA, 29903 bp, Severe acute respiratory syndrome-related coronavirus
  NC_045512.2 Severe acute respiratory syndrome coronavirus 2 isolate Wuhan-Hu-1, complete genome
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 18 - protein, 723 aa, Artificial Sequence
  human Angiotensin-Converting-Enzyme 2 (ACE2)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 19 - protein, 128 aa, Artificial Sequence
  human GM-CSF
MAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE